SEQUENCE LISTING (64 sequences)

SEQ ID NO: 1 - DNA, 2206 bp, Mus musculus
ggcacgaggcttgggcaacatggcggcagttgtgttgctgagcttggactgagcaacagcgagtgtggcgcgctccttcccgggctttggagtgcgtcagcgtgagaagagggactgggtcctggtcttcctgctcctgtcccagcgtcacctgcacctcctgtgtgctctcctctgtcggaaccagagggaatgacgaagcccagggcttttcgcagtggtgcactgtgcatcggaccacgctcctaggcgatagtgggcaagtcttctcgcagtcctctctccaacctccttccgtcggaggtaggaccgaagcgtgggcggttgcgattccccagggaccatggccgggtccgacacggcgcccttcctcagccaagcagatgatcctgatgacgggccagcgcccggccatccggggttgccaggacccatggggaatccaaagtccggggaactcgaggtcccagactgtgaggggctacagcgcatcactggcttatctcggggccattcgaccctcatagtggtggttctgtgctacattaacctcctgaactacatggaccgcttcaccgtggcaggggttcttacagacatcgagcagttctttaacatcggagatggtagtactggcctcatccagactgtgttcatctccagttacatggtgttggcaccagtgtttggctacctgggtgacaggtacaatcgaaagtacctcatgtgcgggggcattgccttctggtccctggtgacactgggatcatccttcatccccagagagcatttctggctgcttctcctgacccggggcctggtgggggtcggggaggccagttactccaccattgcgcccaccctgatcgccgacctcttcgtggcagaccagcggagtcggatgctcagtatcttctactttgccatccctgtgggcagtggtctaggttacattgctggctccaaagtgaaagacgtggctggagactggcactgggctctacgggtgacaccaggtctaggagtgctggctgtcctgctgctgttcctggtggtccaggagcccccaagaggagccgtggagcgccactcaggttcaccacccctgagccccacctcttggtgggcagatctgaaggcactggcacgaaatcctagtttcgtcctgtcttcccttggcttcacctctgtggcctttgtcacgggctccctggctctctgggccccagcgttcctgctgcgctcccgggttgttctgggagagactccgccctgtctccctggagattcatgctcttcctctgacagtctcatctttggactcatcacttgcctgactggagtcctgggtgtgggcctgggagtggagatcagccgccgccttcgccgcttcaaccctcgggctgacccactcgtctgtgcagctggcctcctgggttcggcgcctttcctcttcctggccctggcctgtgcccgaggtagcatcgtggccacctatatttttatctttattggggagaccctgttgtccatgaactgggccattgtggctgacatcctgttgtacgtggtgatcccaactcgacggtccacggctgaggccttccagatagtgctgtcccacttgctaggagatgcagggagcccttacctcattggtctaatctctgaccgcctccgacggagctggcccccttccttcctgtccgagttccgggctctgcagttctcgctcatgctctgtgctttcgttggggcactgggtggtgcggccttcctgggcaccgccatgttcattgaagatgaccgccggcgggctcaactccacgtgcagggtctgttgcatgagtctgggccctcagatgaccggattgtagtacctcagcgaggccgttctacccgagtccccgtgtccagcgtgctcatctgaggagccggtgcttacccggccactgatgcatcgcagctgggccttgggcccacccaagacggttcccaggcagaagccctcaccaggcccaggtccaagaaggaagccctgggatatctcccagctcccagacactacatgggcagcccagggaagagatgggagtccagaaacggggaaggggtgtcctctctactaggacagcccaaggggtttggtgctatttgtaatggaataaaatttgtaatcagaaaaaaaaaaaaaaaaaaaaaaaaa

SEQ ID NO: 2 - DNA, 2206 bp, Mus musculus
ggcacgaggcttgggcaacatggcggcagttgtgttgctgagcttggactgagcaacagcgagtgtggcgcgctccttcccgggctttggagtgcgtcagcgtgagaagagggactgggtcctggtcttcctgctcctgtcccagcgtcacctgcacctcctgtgtgctctcctctgtcggaaccagagggaatgacgaagcccagggcttttcgcagtggtgcactgtgcatcggaccacgctcctaggcgatagtgggcaagtcttctcgcagtcctctctccaacctccttccgtcggaggtaggaccgaagcgtgggcggttgcgattccccagggaccatggccgggtccgacacggcgcccttcctcagccaagcagatgatcctgatgacgggccagcgcccggccatccggggttgccaggacccatggggaatccaaagtccggggaactcgaggtcccagactgtgaggggctacagcgcatcactggcttatctcggggccattcgaccctcatagtggtggttctgtgctacattaacctcctgaactacatggaccgcttcaccgtggcaggggttcttacagacatcgagcagttctttaacatcggagatggtagtactggcctcatccagactgtgttcatctccagtcacatggtgttggcaccagtgtttggctacctgggtgacaggtacaatcgaaagtacctcatgtgcgggggcattgccttctggtccctggtgacactgggatcatccttcatccccagagagcatttctggctgcttctcctgacccggggcctggtgggggtcggggaggccagttactccaccattgcgcccaccctgatcgccgacctcttcgtggcagaccagcggagtcggatgctcagtatcttctactttgccatccctgtgggcagtggtctaggttacattgctggctccaaagtgaaagacgtggctggagactggcactgggctctacgggtgacaccaggtctaggagtgctggctgtcctgctgctgttcctggtggtccaggagcccccaagaggagccgtggagcgccactcaggttcaccacccctgagccccacctcttggtgggcagatctgaaggcactggcacgaaatcctagtttcgtcctgtcttcccttggcttcacctctgtggcctttgtcacgggctccctggctctctgggccccagcgttcctgctgcgctcccgggttgttctgggagagactccgccctgtctccctggagattcatgctcttcctctgacagtctcatctttggactcatcacttgcctgactggagtcctgggtgtgggcctgggagtggagatcagccgccgccttcgccgcttcaaccctcgggctgacccactcgtctgtgcagctggcctcctgggttcggcgcctttcctcttcctggccctggcctgtgcccgaggtagcatcgtggccacctatatttttatctttattggggagaccctgttgtccatgaactgggccattgtggctgacatcctgttgtacgtggtgatcccaactcgacggtccacggctgaggccttccagatagtgctgtcccacttgctaggagatgcagggagcccttacctcattggtctaatctctgaccgcctccgacggagctggcccccttccttcctgtccgagttccgggctctgcagttctcgctcatgctctgtgctttcgttggggcactgggtggtgcggccttcctgggcaccgccatgttcattgaagatgaccgccggcgggctcaactccacgtgcagggtctgttgcatgagtctgggccctcagatgaccggattgtagtacctcagcgaggccgttctacccgagtccccgtgtccagcgtgctcatctgaggagccggtgcttacccggccactgatgcatcgcagctgggccttgggcccacccaagacggttcccaggcagaagccctcaccaggcccaggtccaagaaggaagccctgggatatctcccagctcccagacactacatgggcagcccagggaagagatgggagtccagaaacggggaaggggtgtcctctctactaggacagcccaaggggtttggtgctatttgtaatggaataaaatttgtaatcagaaaaaaaaaaaaaaaaaaaaaaaaa

SEQ ID NO: 3 - protein, 528 aa, Mus musculus
MAGSDTAPFLSQADDPDDGPAPGHPGLPGPMGNPKSGELEVPDCEGLQRITGLSRGHSTLIVVVLCYINLLNYMDRFTVAGVLTDIEQFFNIGDGSTGLIQTVFISSYMVLAPVFGYLGDRYNRKYLMCGGIAFWSLVTLGSSFIPREHFWLLLLTRGLVGVGEASYSTIAPTLIADLFVADQRSRMLSIFYFAIPVGSGLGYIAGSKVKDVAGDWHWALRVTPGLGVLAVLLLFLVVQEPPRGAVERHSGSPPLSPTSWWADLKALARNPSFVLSSLGFTSVAFVTGSLALWAPAFLLRSRVVLGETPPCLPGDSCSSSDSLIFGLITCLTGVLGVGLGVEISRRLRRFNPRADPLVCAAGLLGSAPFLFLALACARGSIVATYIFIFIGETLLSMNWAIVADILLYVVIPTRRSTAEAFQIVLSHLLGDAGSPYLIGLISDRLRRSWPPSFLSEFRALQFSLMLCAFVGALGGAAFLGTAMFIEDDRRRAQLHVQGLLHESGPSDDRIVVPQRGRSTRVPVSSVLI

SEQ ID NO: 4 - protein, 528 aa, Mus musculus
MAGSDTAPFLSQADDPDDGPAPGHPGLPGPMGNPKSGELEVPDCEGLQRITGLSRGHSTLIVVVLCYINLLNYMDRFTVAGVLTDIEQFFNIGDGSTGLIQTVFISSHMVLAPVFGYLGDRYNRKYLMCGGIAFWSLVTLGSSFIPREHFWLLLLTRGLVGVGEASYSTIAPTLIADLFVADQRSRMLSIFYFAIPVGSGLGYIAGSKVKDVAGDWHWALRVTPGLGVLAVLLLFLVVQEPPRGAVERHSGSPPLSPTSWWADLKALARNPSFVLSSLGFTSVAFVTGSLALWAPAFLLRSRVVLGETPPCLPGDSCSSSDSLIFGLITCLTGVLGVGLGVEISRRLRRFNPRADPLVCAAGLLGSAPFLFLALACARGSIVATYIFIFIGETLLSMNWAIVADILLYVVIPTRRSTAEAFQIVLSHLLGDAGSPYLIGLISDRLRRSWPPSFLSEFRALQFSLMLCAFVGALGGAAFLGTAMFIEDDRRRAQLHVQGLLHESGPSDDRIVVPQRGRSTRVPVSSVLI

SEQ ID NO: 5 - DNA, 2165 bp, Homo sapiens
ctgagcgacagcaagtgcagcgggctcctaccccgggtgaggggtggcctccgcgtgggatcgtgccctcttcagcccgctcctgtccccgacatcacgtgtattccgcacgtcccctccgcgctgtgtgtctactgagacggggaggcgtgacagggcccgggtcccttctcagtggtgctctgtgcttcagggcaagctccccgtctccgggcgcacttccctcgcctgtgttcggtccatcctcctttctccagcctcctcccctcgcaggtgggatcgtcggtgggaccggagcgcgggcgggcgcggccccccgggaccatggccgggtccgacaccgcgcccttcctcagccaggcggatgacccggacgacgggccagtgcctggcaccccggggttgccagggtccacggggaacccgaagtccgaggagcccgaggtcccggaccaggaggggctgcagcgcatcaccggcctgtctcccggccgttcggctctcatagtggcggtgctgtgctacatcaatctcctgaactacatggaccgcttcaccgtggctggcgtccttcccgacatcgagcagttcttcaacatcggggacagtagctctgggctcatccagaccgtgttcatctccagttacatggtgttggcacctgtgtttggctacctgggtgacaggtacaatcggaagtatctcatgtgcgggggcattgccttctggtccctggtgacactggggtcatccttcatccccggagagcatttctggctgctcctcctgacccggggcctggtgggggtcggggaggccagttattccaccatcgcgcccactctcattgccgacctctttgtggccgaccagcggagccggatgctcagcatcttctactttgccattccggtgggcagtggtctgggctacattgcaggctccaaagtgaaggatatggctggagactggcactgggctctgagggtgacaccgggtctaggagtggtggccgttctgctgctgttcctggtagtgcgggagccgccaaggggagccgtggagcgccactcagatttgccacccctgaaccccacctcgtggtgggcagatctgagggctctggcaagaaatcctagtttcgtcctgtcttccctgggcttcactgctgtggcctttgtcacgggctccctggctctgtgggctccggcattcctgctgcgttcccgcgtggtccttggggagaccccaccctgccttcccggagactcctgctcttcctctgacagtctcatctttggactcatcacctgcctgaccggagtcctgggtgtgggcctgggtgtggagatcagccgccggctccgccactccaacccccgggctgatcccctggtctgtgccactggcctcctgggctctgcacccttcctcttcctgtcccttgcctgcgcccgtggtagcatcgtggccacttatattttcatcttcattggagagaccctcctgtccatgaactgggccatcgtggccgacattctgctgtacgtggtgatccctacccgacgctccaccgccgaggccttccagatcgtgctgtcccacctgctgggtgatgctgggagcccctacctcattggcctgatctctgaccgcctgcgccggaactggcccccctccttcttgtccgagttccgggctctgcagttctcgctcatgctctgcgcgtttgttggggcactgggcggcgcagccttcctgggcaccgccatcttcattgaggccgaccgccggcgggcacagctgcacgtgcagggcctgctgcacgaagcagggtccacagacgaccggattgtggtgccccagcggggccgctccacccgcgtgcccgtggccagtgtgctcatctgagaggctgccgctcacctacctgcacatctgccacagctggccctgggcccaccccacgaagggcctgggcctaaccccttggcctggcccagcttccagagggaccctgggccgtgtgccagctcccagacactacatgggtagctcaggggaggaggtgggggtccaggagggggatccctctccacaggggcagccccaagggctcggtgctatttgtaacggaataaaatttgtgccagaaaaaaaaaaaa

SEQ ID NO: 6 - DNA, 2165 bp, Homo sapiens
ctgagcgacagcaagtgcagcgggctcctaccccgggtgaggggtggcctccgcgtgggatcgtgccctcttcagcccgctcctgtccccgacatcacgtgtattccgcacgtcccctccgcgctgtgtgtctactgagacggggaggcgtgacagggcccgggtcccttctcagtggtgctctgtgcttcagggcaagctccccgtctccgggcgcacttccctcgcctgtgttcggtccatcctcctttctccagcctcctcccctcgcaggtgggatcgtcggtgggaccggagcgcgggcgggcgcggccccccgggaccatggccgggtccgacaccgcgcccttcctcagccaggcggatgacccggacgacgggccagtgcctggcaccccggggttgccagggtccacggggaacccgaagtccgaggagcccgaggtcccggaccaggaggggctgcagcgcatcaccggcctgtctcccggccgttcggctctcatagtggcggtgctgtgctacatcaatctcctgaactacatggaccgcttcaccgtggctggcgtccttcccgacatcgagcagttcttcaacatcggggacagtagctctgggctcatccagaccgtgttcatctccagtcacatggtgttggcacctgtgtttggctacctgggtgacaggtacaatcggaagtatctcatgtgcgggggcattgccttctggtccctggtgacactggggtcatccttcatccccggagagcatttctggctgctcctcctgacccggggcctggtgggggtcggggaggccagttattccaccatcgcgcccactctcattgccgacctctttgtggccgaccagcggagccggatgctcagcatcttctactttgccattccggtgggcagtggtctgggctacattgcaggctccaaagtgaaggatatggctggagactggcactgggctctgagggtgacaccgggtctaggagtggtggccgttctgctgctgttcctggtagtgcgggagccgccaaggggagccgtggagcgccactcagatttgccacccctgaaccccacctcgtggtgggcagatctgagggctctggcaagaaatcctagtttcgtcctgtcttccctgggcttcactgctgtggcctttgtcacgggctccctggctctgtgggctccggcattcctgctgcgttcccgcgtggtccttggggagaccccaccctgccttcccggagactcctgctcttcctctgacagtctcatctttggactcatcacctgcctgaccggagtcctgggtgtgggcctgggtgtggagatcagccgccggctccgccactccaacccccgggctgatcccctggtctgtgccactggcctcctgggctctgcacccttcctcttcctgtcccttgcctgcgcccgtggtagcatcgtggccacttatattttcatcttcattggagagaccctcctgtccatgaactgggccatcgtggccgacattctgctgtacgtggtgatccctacccgacgctccaccgccgaggccttccagatcgtgctgtcccacctgctgggtgatgctgggagcccctacctcattggcctgatctctgaccgcctgcgccggaactggcccccctccttcttgtccgagttccgggctctgcagttctcgctcatgctctgcgcgtttgttggggcactgggcggcgcagccttcctgggcaccgccatcttcattgaggccgaccgccggcgggcacagctgcacgtgcagggcctgctgcacgaagcagggtccacagacgaccggattgtggtgccccagcggggccgctccacccgcgtgcccgtggccagtgtgctcatctgagaggctgccgctcacctacctgcacatctgccacagctggccctgggcccaccccacgaagggcctgggcctaaccccttggcctggcccagcttccagagggaccctgggccgtgtgccagctcccagacactacatgggtagctcaggggaggaggtgggggtccaggagggggatccctctccacaggggcagccccaagggctcggtgctatttgtaacggaataaaatttgtgccagaaaaaaaaaaaa

SEQ ID NO: 7 - protein, 528 aa, Homo sapiens
MAGSDTAPFLSQADDPDDGPVPGTPGLPGSTGNPKSEEPEVPDQEGLQRITGLSPGRSALIVAVLCYINLLNYMDRFTVAGVLPDIEQFFNIGDSSSGLIQTVFISSYMVLAPVFGYLGDRYNRKYLMCGGIAFWSLVTLGSSFIPGEHFWLLLLTRGLVGVGEASYSTIAPTLIADLFVADQRSRMLSIFYFAIPVGSGLGYIAGSKVKDMAGDWHWALRVTPGLGVVAVLLLFLVVREPPRGAVERHSDLPPLNPTSWWADLRALARNPSFVLSSLGFTAVAFVTGSLALWAPAFLLRSRVVLGETPPCLPGDSCSSSDSLIFGLITCLTGVLGVGLGVEISRRLRHSNPRADPLVCATGLLGSAPFLFLSLACARGSIVATYIFIFIGETLLSMNWAIVADILLYVVIPTRRSTAEAFQIVLSHLLGDAGSPYLIGLISDRLRRNWPPSFLSEFRALQFSLMLCAFVGALGGAAFLGTAIFIEADRRRAQLHVQGLLHEAGSTDDRIVVPQRGRSTRVPVASVLI

SEQ ID NO: 8 - protein, 528 aa, Homo sapiens
MAGSDTAPFLSQADDPDDGPVPGTPGLPGSTGNPKSEEPEVPDQEGLQRITGLSPGRSALIVAVLCYINLLNYMDRFTVAGVLPDIEQFFNIGDSSSGLIQTVFISSHMVLAPVFGYLGDRYNRKYLMCGGIAFWSLVTLGSSFIPGEHFWLLLLTRGLVGVGEASYSTIAPTLIADLFVADQRSRMLSIFYFAIPVGSGLGYIAGSKVKDMAGDWHWALRVTPGLGVVAVLLLFLVVREPPRGAVERHSDLPPLNPTSWWADLRALARNPSFVLSSLGFTAVAFVTGSLALWAPAFLLRSRVVLGETPPCLPGDSCSSSDSLIFGLITCLTGVLGVGLGVEISRRLRHSNPRADPLVCATGLLGSAPFLFLSLACARGSIVATYIFIFIGETLLSMNWAIVADILLYVVIPTRRSTAEAFQIVLSHLLGDAGSPYLIGLISDRLRRNWPPSFLSEFRALQFSLMLCAFVGALGGAAFLGTAIFIEADRRRAQLHVQGLLHEAGSTDDRIVVPQRGRSTRVPVASVLI

SEQ ID NO: 9 - DNA, 149 bp, Artificial Sequence
  microsatellite marker
tcagtgttatgccagtgggaacaagctgctgctgctgctgctgctgctgctgctgctgctgctgctgctgctgctgctgcactgtgaacaaggcatgcttcccaaccttggagtacagttcactgacaggaagtgcaagacaccaggag

SEQ ID NO: 10 - DNA, 22 bp, Artificial Sequence
  primer
tcagtgttatgccagtgggaac

SEQ ID NO: 11 - DNA, 21 bp, Artificial Sequence
  primer
ctcctggtgtcttgcacttcc

SEQ ID NO: 12 - DNA, 597 bp, Artificial Sequence
  microsatellite marker
cattaccaccacacccctgtgacagacagacagacagacagacagacagacacacacacacacacacacacacacacacgtggggggagggagagagagaagacgaagaccaaggagctgactccgccctttctctccacaggacttacttcccatttcgctgtggatggatgaaccaactgtcggctaccattacaaaatggtgaggtgggcagggctgctgggtggagagtccacacggggacttgccaccgcccttctcctttgtcccctctcatcactatgtcatcgcctttgacgtcccctccgcccccaggatcatcatgagaaaaagtctgatccgacacttgcaggaccgaggggtgcaggtgaggactcagctctgccccagcgaggagctggctcacagtgtacgcctgggagcagagtttccgccattctgtgacacctctgagtgtcggttactttgcctataaaatgcagagaataagtatctgcagatcttttggctgtgtacctggtacttaatcagagattggtaagcattagcactggtcaccatgcttgtggccagctgggaacatgctggaaagccag

SEQ ID NO: 13 - DNA, 20 bp, Artificial Sequence
  primer
cattaccaccacacccctgt

SEQ ID NO: 14 - DNA, 19 bp, Artificial Sequence
  primer
ctggctttccagcatgttc

SEQ ID NO: 15 - DNA, 21 bp, Artificial Sequence
  primer
cgattgattacaccagcttgc

SEQ ID NO: 16 - DNA, 20 bp, Artificial Sequence
  primer
catcaggatcatctgcttgg

SEQ ID NO: 17 - DNA, 20 bp, Artificial Sequence
  primer
ctcgcagtcctctctccaac

SEQ ID NO: 18 - DNA, 20 bp, Artificial Sequence
  primer
aagagccctggccactaaag

SEQ ID NO: 19 - DNA, 20 bp, Artificial Sequence
  primer
gatggtgcgacatcagtgag

SEQ ID NO: 20 - DNA, 20 bp, Artificial Sequence
  primer
agaggacaatggctgtctgc

SEQ ID NO: 21 - DNA, 20 bp, Artificial Sequence
  primer
gattccagccaggtctgtgt

SEQ ID NO: 22 - DNA, 20 bp, Artificial Sequence
  primer
gaccctcgagtcaaggagaa

SEQ ID NO: 23 - DNA, 20 bp, Artificial Sequence
  primer
ccccagcgtctcagaactta

SEQ ID NO: 24 - DNA, 20 bp, Artificial Sequence
  primer
tgaatctccagggagacagg

SEQ ID NO: 25 - DNA, 20 bp, Artificial Sequence
  primer
tgtggggtctgacttcctct

SEQ ID NO: 26 - DNA, 19 bp, Artificial Sequence
  primer
cacagacgagtgggtcagc

SEQ ID NO: 27 - DNA, 20 bp, Artificial Sequence
  primer
cctctaccccacccctagtc

SEQ ID NO: 28 - DNA, 20 bp, Artificial Sequence
  primer
aaatctgggtggcagaagtg

SEQ ID NO: 29 - DNA, 20 bp, Artificial Sequence
  primer
ggtgggaagtggtactgagg

SEQ ID NO: 30 - DNA, 20 bp, Artificial Sequence
  primer
atgcgccataggcatctatt

SEQ ID NO: 31 - DNA, 20 bp, Artificial Sequence
  primer
gttttggggacctggaactc

SEQ ID NO: 32 - DNA, 20 bp, Artificial Sequence
  primer
cccaaagctaagctggatga

SEQ ID NO: 33 - DNA, 20 bp, Artificial Sequence
  primer
ggattgggctctgtctgtgt

SEQ ID NO: 34 - DNA, 20 bp, Artificial Sequence
  primer
gacagggacttacccacagg

SEQ ID NO: 35 - DNA, 137 bp, Murinae gen. sp.
tgttcatctccagttacatggtgttggcaccagtgtttggctacctgggtgacaggtacaatcgaaagtacctcatgtgcgggggcattgccttctggtccctggtgacactgggatcatccttcatccccagagag

SEQ ID NO: 36 - DNA, 137 bp, Murinae gen. sp.
  exon 3
tgttcatctccagtcacatggtgttggcaccagtgtttggctacctgggtgacaggtacaatcgaaagtacctcatgtgcgggggcattgccttctggtccctggtgacactgggatcatccttcatccccagagag

SEQ ID NO: 37 - DNA, 20 bp, Artificial Sequence
  primer
gtctaggagtgctggctgtc

SEQ ID NO: 38 - DNA, 20 bp, Artificial Sequence
  primer
gatatcccagggcttccttc

SEQ ID NO: 39 - DNA, 300 bp, Mus sp.
  misc_feature: Northern Probe
gtctaggagtgctggctgtcctgctgctgttcctggtggtccaggagcccccaagaggagccgtggagcgccactcaggttcaccacccctgagccccacctcttggtgggcagatctgaaggcactggcacgaaatcctagtttcgtcctgtcttcccttggcttcacctctgtggcctttgtcacgggctccctggctctctgggccccagcgttcctgctgcgctcccgggttgttctgggagagactccgccctgtctccctggagattcatgctcttcctctgacagtctcatct

SEQ ID NO: 40 - protein, 528 aa, Rattus norvegicus
MAGSDTAPFLSQADDPDDGPAPGHPGLPGPMGNPKSGELEVPDCEGLQRITGLSRGHSTLIVVVLCYINLLNYMDRFTVAGVLTDIEQFFNIGDGSTGLIQTVFISSYMVLAPVFGYLGDRYNRKYLMCGGIAFWSLVTLGSSFIPREHFWLLLLTRGLVGVGEASYSTIAPTLIADLFVADQRSRMLSIFYFAIPVGSGLGYIAGSKVKDLAGDWHWALRVTPGLGVLAVLLLFLVVQEPPRGAVERHSGSPPLSPTSWWADLKALARSPSFVLSSLGFTAVAFVTGSLALWAPAFLLRSRVVLGETPPCLPGDSCSSSDSLIFGLITCLTGVLGVGLGVEISRRLRRFNPRADPLVCAAGLLGSSPFLFLSLACARGSIVATYIFIFIGETLLSMNWAIVADILLYVVIPTRRSTAEAFQIVLSHLLGDAGSPYLIGLISDRLRRSWPPSFLSEFRALQFSLMLCAFVGALGGAAFLGTAMFIENDRRRAQLHVQGLLHETEPSDDQIVVPQRGRSTRVPVSSVLI

SEQ ID NO: 41 - protein, 506 aa, Danio rerio
MSQADADITPFFADDNEGEGPVENGVGSPLPEDEEEESPSGVTDRRAIMTVIVLCYINLLNYMDRFTVAGVLPDIEHFFGIGDGTSGLLQTVFICSYMFLAPLFGYLGDRYNRKLIMCVGIFFWSVVTLASSFIGKDHFWALLLTRGLVGVGEASYSTIAPTIIADLFVKEKRTNMLSIFYFAIPVGSGMGYIVGSKVDTVAKDWHWALRVTPGLGLLAVFLLMLVVQEPKRGAIEAHPEHTLHRTSWLADMKALCRNPSFILSTFGFTAVAFVTGSLALWAPAFLFRAGVFTGVKQPCFKAPCDDSDSLIFGAITVVTGILGVASGVQASKLLRTRTPRADPLVCAAGLLLAAPFLYLSIIFAQASTVATYVFIFLGETFLSMNWAIVADILLYVVIPTRRSTAEAFQIVLSHLLGDAISPYLIGVVSDSIKESNSYMWEFRSLQMSLLLCSFVAVAGGAFFLATAVFIEKDRDLAENYVPSDDAPIVVPRSGRSTKVSVSSVLI

SEQ ID NO: 42 - protein, 556 aa, Fugu rubripes
VLSPVFICSYMFLAPVFGYLGDRYNRKFIMSAGIAFWSVVTLASSYTPGAHFWLLLLTRGLVGVGEASYSTIAPTVIADLYVKETRTNMLSLFYFAIPVGSGLGYIVGSQVGSLAGDWHWALRVTPGLGLVAVLLLLLVVQEPRRGAVERPHRQVRRTGWLTDLSALSRNHSFLLSTFGFTAVAFVTGSLALWAPTFLFRAAVFTGERAPCVAGNCAASDSLLFGAITCVTGVLGVASGVQVSRLLRRRTGRADPLVCAAGLLLSAPFLYLAVVFAQASTVATYVFIFFGETFLSMNWAIVADILLYVVVPTRRATAEALQIVVSHLLGDAGSPYLIGVVSDTLRRSDSFLWRFRSLQLSLLLCSFVAVVGGAFFLATALFIETDRHRAETYDTAGDEPIVVPKSGRSTRVPVSSVLIVATYVFIFFGETFLSMNWAIVADILLYVVVPTRRATAEALQIVVSHLLGDAGSPYLIGVVSDTLRRSDSFLWRFRSLQLSLLLCSFVAVVGGAFFLATALFIETDRHRAETYDTAGDEPIVVPKSGRSTRVPVSSVLI

SEQ ID NO: 43 - DNA, 21 bp, Murinae gen. sp.
aagcagatgatcctgatgacg

SEQ ID NO: 44 - DNA, 21 bp, Murinae gen. sp.
aggacccatggggaatccaaa

SEQ ID NO: 45 - DNA, 21 bp, Murinae gen. sp.
gctacattaacctcctgaact

SEQ ID NO: 46 - DNA, 21 bp, Murinae gen. sp.
aaggcactggcacgaaatcct

SEQ ID NO: 47 - DNA, 20 bp, Murinae gen. sp.
agcatcgtggccacctatat

SEQ ID NO: 48 - DNA, 21 bp, Murinae gen. sp.
agcccttacctcattggtcta

SEQ ID NO: 49 - DNA, 63 bp, Artificial Sequence
  Synthetically generated oligonucleotide
gatccgcagatgatcctgatgacgttcaagagacgtcatcaggatcatctgcttttttggaaa

SEQ ID NO: 50 - DNA, 63 bp, Artificial Sequence
  Synthetically generated oligonucleotide
agcttttccaaaaaagcagatgatcctgatgacgtctcttgaacgtcatcaggatcatctgcg

SEQ ID NO: 51 - DNA, 65 bp, Artificial Sequence
  Synthetically generated oligonucleotide
gatccggacccatggggaatccaaattcaagagatttggattccccatgggtccttttttggaaa

SEQ ID NO: 52 - DNA, 65 bp, Artificial Sequence
  Synthetically generated oligonucleotide
agcttttccaaaaaaggacccatggggaatccaaatctcttgaatttggattccccatgggtccg

SEQ ID NO: 53 - DNA, 67 bp, Artificial Sequence
  Synthetically generated oligonucleotide
gatccgctacattaacctcctgaactttcaagagaagttcaggaggttaatgtagcttttttggaaa

SEQ ID NO: 54 - DNA, 67 bp, Artificial Sequence
  Synthetically generated oligonucleotide
agcttttccaaaaaagctacattaacctcctgaacttctcttgaaagttcaggaggttaatgtagcg

SEQ ID NO: 55 - DNA, 63 bp, Artificial Sequence
  Synthetically generated oligonucleotide
gatccggcactggcacgaaatcctttcaagagaaggatttcgtgccagtgccttttttggaaa

SEQ ID NO: 56 - DNA, 63 bp, Artificial Sequence
  Synthetically generated oligonucleotide
agcttttccaaaaaaggcactggcacgaaatccttctcttgaaaggatttcgtgccagtgccg

SEQ ID NO: 57 - DNA, 63 bp, Artificial Sequence
  Synthetically generated oligonucleotide
gatccgcatcgtggccacctatatttcaagagaatataggtggccacgatgcttttttggaaa

SEQ ID NO: 58 - DNA, 63 bp, Artificial Sequence
  Synthetically generated oligonucleotide
agcttttccaaaaaagcatcgtggccacctatattctcttgaaatataggtggccacgatgcg

SEQ ID NO: 59 - DNA, 65 bp, Artificial Sequence
  Synthetically generated oligonucleotide
gatccgcccttacctcattggtctattcaagagatagaccaatgaggtaagggcttttttggaaa

SEQ ID NO: 60 - DNA, 65 bp, Artificial Sequence
  Synthetically generated oligonucleotide
agcttttccaaaaaagcccttacctcattggtctatctcttgaatagaccaatgaggtaagggcg

SEQ ID NO: 61 - protein, 528 aa, Artificial Sequence
  Consensus sequence
MAGSDTAPFLSQADDPDDGPAPGHPGLPGPMGNPKSEELEVPDCEGLQRITGLSRGRSALIVAVLCYINLLNYMDRFTVAGVLPDIEQFFNIGDGSSGLIQTVFISSYMVLAPVFGYLGDRYNRKYLMCGGIAFWSLVTLGSSFIPREHFWLLLLTRGXVGVGEASYSTIAPTLIADLFVADQRSRMLSIFYFAIPVGSGLGYIAGSKVKDMAGDWHWALRVTPGLGVLAVLLLFLVVREPPRGAVERHSDLPPLNPTSWWADLRALARNPSFVLSSLGFTAVAFVTGSLALWAPAFLLRSRVVLGETPPCLPGDSCSSSDSLIFGLITCLTGVLGVGLGMEISRRLRRFNPRADPLVCAAGLLGSAPFLFLALACARGSIVATYIFIFIGETLLSMNWAIVADILLYVVIPTRRSTAEAFQIVLSHLLGDAGSPYLIGLISDRLRRNWPPSFLSEFRALQFSLMLCAFVGALGGAAFLGTAIFIEADRRRAQLHVQGLLHEAGPSDDRIVVPQRGRSTRVPVASVLI

SEQ ID NO: 62 - protein, 525 aa, Artificial Sequence
  Consensus sequence
MAGSDTAPFLSQADDPDDGPAPGHPGLPGPMGNPKSGELEVPDCEGLQRITGLSRGHSTLIVVVLCYINLLNYMDRFTVAGVLTDIEQFFNIGDGSTGLIQTVFISSYMVLAPVFGYLGDRYNRKYLMCGGIAFWSLVTLGSSFIPREHFWLLLLTRGXVGVGEASYSTIAPTLIADLFVADQRSRMLSIFYFAIPVGSGLGYIAGSKVKDAGDWHWALRVTPGLGVLAVLLLFLVVQEPPRGAVERHSGSPPLSPTSWWADLKALARNPSFVLSSLGFTAVAFVTGSLALWAPAFLLRSRVVLGETPPCLPGDSCSSSDSLIFGLITCLTGVLGVGLGVEISRRLRRFNPRADPLVCAAGLLGSAPFLFLSLACARGSIVATYIFIFIGETLLSMNWAIVADILLYVVIPTRRSTAEAFQIVLSHLLGDAGSPYLIGLISDRLRRSWPPSFLSEFRALQFSLMLCAFVGALGGAAFLGTAMFIEDRRRAQLHVQGLLHEGPSDDRIVVPQRGRSTRVPVSSVLI

SEQ ID NO: 63 - protein, 506 aa, Artificial Sequence
  Consensus sequence
MAGSDTAPFLSXADXPDXGPVPGPGLPGGNPKSEEEVPDEGLQRITGLSGRSXIVVLCYINLLNYMDRFTVAGVLPDIEQFFNIGDGSSGLIQTVFISSYMVLAPVFGYLGDRYNRKYMCGGIAFWSLVTLGSSFIPXHFWLLLLTRGXVGVGEASYSTIAPTLIADLFVAXQRSRMLSIFYFAIPVGSGXGYIAGSKVKDVAGDWHWALRVTPGLGVLAVLLLFLVVQEPPRGAXERHSPPLPTSWWADXKALARNPSFXLSSLGFTAVAFVTGSLALWAPAFLLRSRVVLGETPPCLPGDSCSSSDSLIFGLITCLTGXLGVGLGVXISRRLRNPRADPLVCAAGLLGSAPFLXLSLACARGSIVATYXFIFIGETLLSMNWAIVADILLYVVIPTRRSTAEAFQIVLSHLLGDAGSPYLIGLXSDRLRRSWPPSXXSEFRALQFSLXLCAFVGALGGAALGTAFIEDRRRAXLHVQGLLHESDDIVVPQRGRSTRVPVSSVLI

SEQ ID NO: 64 - protein, 420 aa, Artificial Sequence
  Consensus sequence
ADPFDGPGPPEEPGVVLCYINLLNYMDRFTVAGVLDIEFFIGDGGLLQTVFICSYMFLAPVFGYLGDRYNRKIMCGIAFWSVVTLASSXIPHFWLLLLTRGXVGVGEASYSTIAPTIADLXVKXRTNMLSIFYFAIPVGSGXGYIVGSKVAGDWHWALRVTPGLGLLAVLLLLVVQEPRGAXERHHLRTSWLADXKALRNPSFLSTFGFTAVAFVTGSLALWAPAFLFRAVFTGEPCGCSDSLIFGAITCVTGXLGVASGVXSRLLRRRTPRADPLVCAAGLLLSAPFLXLSFAQASTVATYXFIFGETFLSMNWAIVADILLYVVXPTRRSTAEAFQIVLSHLLGDAGSPYLIGVXSDLRRSSXXWEFRSLQSLXLCSFVAVGGALATAFIEDRRAXYVDPIVVPSGRSTRVPVSSVLI

(19) United States
(12) Patent Application Publication
(10) Pub. No.: US 2005/0020527 A1
(43) Pub. Date: Jan. 27, 2005

(54) Spinster-like protein genes, expression products, non-human animal model: uses in human metabolic disorders

(76) Inventors: Thomas Peters, Martinsried (DE); Volker Schluter, Martinsried (DE); Johannes Grosse, Martinsried (DE); Heike Schauerte, Martinsried (DE); Andreas Marquardt, Martinsried (DE)

Correspondence Address: FISH & RICHARDSON PC, 225 FRANKLIN ST, BOSTON, MA, 02110, US

(21) Appl. No.: 10/818,939

(22) Filed: Apr. 5, 2004

Related U.S. Application Data
(60) Provisional application No. 60/460,310, filed on Apr. 4, 2003.
(60) Provisional application No. 60/538,831, filed on Jan. 23, 2004.
(60) Provisional application No. 60/550,192, filed on Mar. 4, 2004.
(60) Provisional application No. 60/550,800, filed on Mar. 5, 2004.

(51) Int. Cl. A61K 48/00

(52) U.S. Cl. 514/44; 800/3

(57) ABSTRACT

The present invention relates to a non-human vertebrate animal model displaying an alteration in fat metabolism or in the sensitivity towards leptin or insulin, which model bears a mutation in the gene encoding the spinster like 1 protein (Spinl1). The invention also relates to mutant Spinl1 proteins and nucleic acid sequences encoding these proteins. Furthermore, the invention relates to the use of the non-human vertebrate animal model for the identification of diagnostic markers, or as a model for studying the molecular and physiological mechanisms associated with an alteration in fat metabolism or an alteration in the sensitivity towards leptin or insulin, or for the identification and testing of agents useful in the prevention, amelioration, or treatment of the above conditions. Agents, pharmaceutical compositions, and methods for treating the above conditions are likewise described, as are methods for identifying said agents.

### FIELD OF THE INVENTION

The present invention relates to a non-human vertebrate animal model with an alteration in fat metabolism, particularly a reduction in fat storage and/or an alteration in liver function. This animal model bears a mutation in the spinster like 1 protein (Spinl1).

The invention also relates to mutant Spinl1 proteins and nucleic acid sequences encoding these proteins.

Furthermore, the invention relates to the use of the non-human vertebrate animal for the identification of diagnostic markers, or as an animal model for studying the molecular and physiological mechanisms associated with an alteration in fat metabolism or with altered activity or expression of endogenous Spinl1, or for the identification and testing of an agent useful in the prevention, amelioration, or treatment of a medical condition associated with an alteration in fat metabolism.

In addition, the invention also relates to the use of agents and pharmaceutical compositions suitable for the modulation of Spinl1 activity in medical conditions associated with an alteration in fat metabolism. Methods of treating said conditions, and methods of identifying said agents are likewise provided. These and further aspects of the invention will be described in more detail below.

Furthermore, the invention relates to methods for screening of agents, capable to functioning as modulators of sensitivity towards leptin (leptin sensitizers or desensitizers) or modulators of leptin activity.

The invention also relates to the use of modulators of sensitivity towards leptin or leptin activity and of Spinl1 and Spinl1 related agents for the treatment of diseases associated with altered serum leptin levels or altered leptin sensitivity.

Also, the invention relates to methods for screening of agents, capable of functioning as modulators of sensitivity towards insulin (insulin sensitizer or desensitizer) or modulators of insulin activity.

In addition, the invention relates to the use of modulators of sensitivity towards insulin or insulin activity, i.e., modulators that interfere with insulin mediated activation of signal transduction, and of Spinl1 and Spinl1 related agents for the treatment of diseases associated with altered plasma insulin levels or altered insulin sensitivity.

### BACKGROUND OF THE INVENTION

The metabolism of a mammalian organism is closely controlled by a complex interplay between central and local regulatory mechanisms. This regulatory network defines the uptake, distribution, local availability and use of nutrients according to the needs of the organism. Disturbance of these processes, either by genetic or environmental influences, leads to a large variety of metabolic changes. From a public health perspective the most important group of metabolic diseases is obesity and type II diabetes mellitus (type II DM), also called non-insulin dependent diabetes mellitus (Tanizawa, Riggs, Chiu, Janssen, Bell, Go, Roseman, Acton, and Permutt 1994). Obesity will be the leading cause of death and disability in this century, according to the WHO.

Both diseases have reached epidemic dimensions in the United States and Western Europe and threaten the population to cause extensive secondary disease burdens. Despite public health efforts, the problem will increase over the next 10 years; the number of obese and overweight people in US and Europe is expected to grow from 95 million in 2000 to 139 million in 2010.

Obese people suffer from an increased risk of developing secondary illnesses, like hypertension, lipid disorders, type II diabetes, coronary heart disease, osteoarthritis, sleep apnoea, respiratory problems, and certain cancers. Less than 5% of potential patients are treated with pharmacological therapies. The modest efficacy of available therapies contributes to the phenomenon that only 5-10% lose initial body weight and further develop cardiovascular or gastrointestinal side effects.

Diabetes mellitus (DM) is a progressive and chronic endocrine disorder primarily resulting in a hyperglycemic (excess glucose in the blood) condition. This condition additionally affects the body's ability to metabolize fat, carbohydrates, and proteins. In non-insulin dependent diabetes mellitus (NIDDM, type II DM), affected individuals have a physiological resistance to the effects of insulin within peripheral tissues. Basically, their body is still capable of producing insulin but the insulin is not physiologically effective.

An individual can be predisposed to NIDDM by both genetic and environmental factors. NIDDM is a polygenic disorder, characterized by gene-gene and gene-environment interactions with an onset in the adulthood, usually at the age of 40 to 60, but occasionally already during adolescence, if a person is obese. Mutations in the following genes have been observed in NIDDM patients: HNF4A, HNF1-alpha, HNF1B, MAPK8IP1, NEUROD1, GPD2, GLUT4, and GLUT2 (Furuta, Furuta, Sanke, Ekawa, Hanabusa, Nishi, Sasaki, and Nanjo 2002; Hegele, Cao, Harris, Hanley, and Zinman 1999; Novials, Vidal, Franco, Ribera, Sener, Malaisse, and Gomis 1997; Waeber, Delplanque, Bonny, Mooser, Steinmann, Widmann, Maillard, Miklossy, Dina, Hani, Vionnet, Nicod, Boutin, and Froguel 2000; Tanizawa, Riggs, Chiu, Janssen, Bell, Go, Roseman, Acton, and Permutt 1994; Kusari, Verma, Buse, Henry, and Olefsky 1991; Hani, Suaud, Boutin, Chevre, Durand, Philippi, Demenais, Vionnet, Furuta, Velho, Bell, Laine, and Froguel 1998). Overwhelming evidence exists that the genetic components can be controlled by following a comprehensive and enduring treatment protocol. The etiology and risk factors for NIDDM have been well established for many years. Hereditary influences, obesity, and increased age play a major role in the onset of NIDDM. Risk factors include prolonged stress, pregnancy, sedentary lifestyle, and certain medications affecting hormonal processes within the body. However, eighty percent or more of the people with NIDDM are obese with the remaining twenty percent considered above ideal weight This indicates a predominant link between obesity and the development of NIDDM. Although the specific physiological causes remain unknown, medical studies revealed that the heavier an individual is, the more insulin is needed to metabolize the food consumed by the individual.

NIDDM has potentially disastrous long-term effects on the body. These can at first manifest as minor annoyances but then insidiously destroy the tissue(s) of a given body part, an organ, or an entire system as is demonstrated, e.g., by diabetic ulceration. Moreover, NIDDM progresses aggressively and the prognosis is poor unless the disease is strictly controlled. Currently, DM is most often treated with diet and physical exercise, typically in combination with oral hyperglycemic drugs (OHD) as no cure is known for DM. Even with proper medical management, the prognosis is still poor due to irreversible major impairments or severe disabilities.

Recently, new drugs for the treatment of these complex diseases have become available. However, these drugs only target a limited spectrum of pathomechanisms among those thought to be important for the development of diabetes and obesity and further investigation of those pathomechanisms is necessary to identify novel therapeutic approaches in the treatment of these diseases.

Pathomechanisms contributing to the development of diabetes and obesity are lipotoxicity, insulin resistance, dysregulation of nutrition sensing, metabolic rate and food intake.

The adipocyte-secreted hormone leptin appears to play a central role in the regulation of metabolic balance and food intake; for review, see (Holash, Wiegand, and Yancopoulos 1999). Leptin also protects non-adipose tissues from lipid overload during phases of overnutrition. Consequently, leptin resistance results in the deposition of fat in non-adipose tissues, such as insulin-producing pancreatic cells, skeletal muscle and heart muscle. High levels of triglycerides in muscle cells result in the accumulation of long chain fatty acyl CoA intermediates that are believed to cause insulin resistance. These intermediates also enter non-oxidative pathways leading to the production of toxic, apoptosis-mediating metabolites, such as ceramides. These effects are believed to be responsible for the depletion of insulin-producing cells in late stages of type II diabetes. It is only partially known which lipids and lipid classes exert these effects and which enzymes are responsible for their synthesis (Prchal and Prchal 1999; Vinores, Derevjanik, Vinores, Okamoto, and Campochiaro 2000; Yuan, Chen, Dellian, Safabakhsh, Ferrara, and Jain 1996). The importance of intracellular triglyceride levels for insulin and leptin sensitivity has been shown in mice with a disrupted gene for Acyl CoA:diacylglycerol transferase 1 (Kerbel 2000). These mice have an increased sensitivity for insulin and leptin and are resistant to diet-induced obesity.

The ratio of saturated to unsaturated fatty acids influences the metabolic balance of the organism. This has been shown by crossing stearoyl CoA desaturase deficient mice to a leptin deficient background. The metabolic rate of the resulting animals was elevated, partially rescuing the morbid obesity resulting from the leptin deficiency (Cohen, Miyazaki, Socci, Hagge-Greenberg, Liedtke, Soukas, Sharma, Hudgins, Ntambi, and Friedman 2002). The molecular basis for this effect is not clear at the moment, but the central regulation of the metabolic rate and physical activity of these animals play an important role.

A therapeutic strategy in the treatment of obesity is to inhibit fatty acid synthase with C75 or its progenitor cerulenin. Application of these compounds resulted in sustained weight loss in leptin deficient mice. At least in part this weight loss is caused by an elevation of the metabolic rate. Inhibition of the fatty acid synthase results in the accumulation of the enzyme's substrate, malonyl CoA (Shimokawa, Kumar, and Lane 2002). Malonyl CoA is an arosteric inhibitor of camitine palmitoyltransferase (CPT) I, the key enzyme that regulates transfer of long-chain fatty acyl-CoA into the mitochondria for β-oxidation. It functions like a switch avoiding β-oxidation and fatty acid synthesis to occur in parallel.

Inhibition of fatty acid synthase is just one possibility to influence the intracellular malonyl CoA level. In a physiological context, feeding cells with glucose and insulin leads likewise to the accumulation of malonyl CoA thereby downregulating β-oxidation. This suggests a key role for malonyl CoA in nutrition sensing, a general mechanism by which cells coordinate fuel utilization depending on fuel supply and metabolic requirements.

Malonyl CoA is produced from acetyl CoA through acetyl CoA carboxylase 2 (Carmeliet and Jain 2000). Deletion of this enzyme results in an increased rate of β-oxidation mediated by the disinhibition of the camitine palmitoyltransferase. The animals are resistant to diet induced obesity and show a reduced fat mass (Abu-Elheiga, Matzuk, Abo-Hashema, and Wakil 2001). The central regulation of energy expenditure and feeding behavior seems to be regulated by intraneuronal malonyl CoA levels (Reischl, Dubois, Peiritsch, Brown, Wheat, Woisetschlager, and Mudde 2000). Clearly, other lipid precursors and intermediates as well as intracellular glucose levels are involved in the regulation of nutrition sensing.

In recent publications leptin has been described to be causative for or to be involved in diseases, which occur independent of obesity. Stenvinkel et al. described leptin to be involved in chronic kidney disease, with elevated leptin levels detected in uremic patients (2003). Schulz et al. described experimental data with the cell surface receptor LDL/A2MR, one genetic factor influencing the development and progression of coronary atherosclerosis. The receptor was shown to be involved in a variety of biological processes leading to atherosclerotic plaque formation. Leptin, a ligand of LDL/A2MR, stimulated LDL/A2MR mRNA and protein expression, as observed in disease-related ex vivo studies (2003). Holtkamp et al. published data of anorexia nervosa (AN) patients, correlating excessive physical activity with low leptin levels. Hypoleptinemia may be one important factor underlying the excessive physical activity of AN patients (2003). Leptin is considered to have a role in the prevention of osteoporosis and probably acts on bone tissue through inhibition of osteoclasia. Coen et al. reported the finding of a positive relation between leptin level and body mass index (BMI), and greater levels in women compared with men. Serum leptin level is reported to be connected to bone resorption and also bone formation, both inversely related to serum leptin levels. Decrease in osteoclasia is accompanied by increasing serum leptin level (2003). Bernstein and Leslie considered low circulating leptin in response to weight loss in any gastrointestinal disease as an important factor in reducing bone mass (2003). Leptin is known to have cardiovascular bioactivity. Barouch et al. described antihypertrophic effects of leptin on the heart (2003). Ozturk et al. published data providing a positive correlation between severity of abstructive sleep apnea and plasma leptin levels (2003). Age-related maculopathy (ARM) or degeneration (ARMD) is the leading cause of irreversible blindness in developed countries. Evereklioglu et al. reported a direct correlation between decreasing leptin levels and severity of maculopathy. Leptin seems to be a possible newly associated factor in the course of ARM and may be involved in the lipid composition of the macular lesions, especially in late-stage ARMD (2003). Investigation of patients with prostate cancer implicated roles of leptin in the development of prostate cancer through testosterone and factors related to obesity (Saglam et al., 2003). Medical strategies in the treatment of obesity also aim at targeting molecules that regulate food intake centrally, comprising ciliary neurotrophic factor (CNTF); neuropeptide Y; the melanocortin-receptor system; melanin-concentrating hormone receptor (MCHR); galanin; orexin A; the serotonin system; noradrenergic receptors alpha1, alpha2, beta2; histamine A3 receptor; leptin; leptin receptor, and sensitizers of the leptin pathway.

It seems to become a recurring theme that small molecule intermediates occurring in the synthesis or metabolism of nutrients are used as indicators of the metabolic state of the whole system. They might act as allosteric modifiers of the activity of enzymes. Long chain fatty acids are supposed to influence the activity of ATP dependent potassium channels thus modifying the activity of pancreatic β-cells or hypothalamic neurons involved in the regulation of feeding behavior. Metabolic pathways used for energy supply in muscle cells serve as nutrition sensor in the hypothalamus, thus generating a close link between metabolism and its regulation (Abu-Elheiga, Matzuk, Abo-Hashema, and Wakil 2001; Shimokawa, Kumar, and Lane 2002).

Recently, a novel mechanism for the regulation of function of nucleocytoplasmic proteins has emerged. Thus, high blood glucose levels result in an elevation of intracellular glucose causing increased glycosylation of signal transduction proteins and transcription factors on serine and threonine residues. Glycosylation with O-linked β-N-acetylglucosamine of these proteins has been shown to regulate their function in a manner similar to phosphorylation (Campochiaro 2000), i.e., glycosylation of insulin receptor substrate 1 has been proposed to reduce its phosphorylation, thereby rendering the insulin signaling pathway insensitive (Carmeliet 2000). It is currently unknown whether the glycosylation of further signaling components plays a role for the development of insulin resistance.

Glycosylation of proteins involved in signaling cascades is one example for the connection between nutrition sensing and the mediation of the cellular response. Other mechanisms connecting these different kinds of intracellular signaling cascades are predicted as glycosylation is obviously specifically connected to glucose metabolism, whereas nutrition sensing refers to a much broader field (Dunn-Meynell, Routh, Kang, Gaspers, and Levin 2002; Song, Levin, McArdle, Bakhos, and Routh 2001).

The Spinster (Spin) gene and protein was first described in Drosophila (Yamamoto, Jallon, and Komatsu 1997; Nakano, Fujitani, Kurihara, Ragan, Usui-Aoki, Shimoda, Lukacsovich, Suzuki, Sezaki, Sano, Ueda, Awano, Kaneda, Umeda, and Yamamoto 2001).

A murine orthologue protein, called Spinster like protein (Spinl) is deposited at NCBI database as Mus musculus spinster-like protein mRNA and amino acid sequence (Genbank Accession No. AF212372 and AAG43831, respectively). Consequently, the amino acid sequence of SEQ ID NO:3 of the present invention and AAG43831 is referred to herein as mouse Spinl1.

Human Spinl1 is also a known gene, deposited at NCBI database as Homo sapiens spinster-like protein mRNA and amino acid sequence (Genbank Accession No. AF212371; and AAG43830, respectively). WO 02/055701 discloses novel human transporter proteins, including a sequence identified as protein 46455 (sequence ID 5), which corresponds to human Spinl1 and is suggested to be a member of the sugar transporter family. Functional data are not provided. WO 01/49728 discloses a large number of nucleotide and amino acid sequences derived from database searches including sequences (identified as sequences ID 116 and 126), with high homology to human Spinl1. The amino acid identity between sequence ID 126 and SEQ ID NO:7 is 90%, due to a gap in sequence ID 126, comprising amino acid positions 271 to 322 of SEQ ID NO:7.

Spinl1 seems to be a membrane protein with an overall structure of a transmembrane transporter with highest similarity to glucose transporters as twelve transmembrane domains (TM1 to TM12) are predicted for mouse spinl1 protein, according to Ensembl Peptide ID ENSMUSP00000032994 (Ensembl Gene ID ENSMUSG00000030741 for the corresonding mouse Spinl1 gene).

Orthologue Spinl1 genes and proteins from other species are referred to herein as, e.g., human Spinl1, fugu Spinl1, or zebrafish Spinl1, respectively.

An Ensembl Protein Family ID ENSF00000000978 currently identifies three members of the mouse Spinl protein family. ENSEMBL is an automatic annotation software program developed by EMBL and the Sanger Institute (hppt://www.ensembl.org). Besides Spinl1, the other proteins of Ensembl protein family ID ENSF00000000978 are ENSMUSP00000044418 (Ensembl Gene ID ENSMUSG0000040447; referred to as mouse Spinl2 below) and ENSMUSP00000021154 (Ensembl Gene ID ENSMUSG0000020798; referred to as mouse Spinl3 below).

The mouse Spinl1 gene is located at chromosome 7 and encodes a protein of 528 amino acids in size. Both, mouse Spinl2 and mouse Spinl3, are located at chromosome 11. Spinl2 encodes a protein of 577 amino acid residues, whereas Spinl3 encodes a protein of 514 amino acids in size. No functional annotation is provided for Spinl1, Spinl2, and Spinl3 by the Ensembl-database.

Non-mammalian animal models bearing Spinl1 mutations have been described for Drosophila and Zebrafish. The name spinster derived from a genetic screen in Drosophila, due to females vigorous rejection to male courtship behavior (Yamamoto, Jallon, and Komatsu 1997). The nervous system of these animals shows several changes possibly explaining this behavioral phenotype. During normal development the abdominal ganglion is reduced in its length due to programmed cell death of neurons. In mutant animals this reduction does not occur due to inhibition of programmed cell death resulting in an abnormally elongated abdominal ganglion. Programmed cell death is also reduced in ovarial nurse cells resulting in a reduced oviposition rate. Furthermore, neurons of adult mutant animals accumulate autofluorescent lipofuscin like material. This accumulation is associated with a moderate degree of neurodegeneration (Nakano, Fujitani, Kurira, Ragan, Usui-Aoki, Shimoda, Lukacsovich, Suzuki, Sezaki, Sano, Ueda, Awano, Kaneda, Umeda, and Yamamoto 2001; Usui-Aoki, Nakano, and Yamamoto 2002). Similar deposits are known from ceroid lipofuscinoses and other lysosomal storage diseases.

In a genetic screen for altered neuromuscular junction morphology another Spinl mutation in Drosophila was detected. The increased area and bouton count of the neuromuscular junction was shown to be caused by increased TGF-β signaling (Sweeney and Davis 2002). The TGF-β receptor is known to stay active upon endocytosis. Inhibition of active ligand receptor complexes is achieved by targeting receptors to lysosomes for degradation. The Spinl protein is restricted to late endosomal and lysosomal compartments. These compartments were shown to be enlarged in Spinl mutant flies pre- and postsynaptically. Enlargement of the late endosomal and lysosomal compartments and dysformation of the subsynaptic reticulum are regarded as causal for the increase in TGF-B signal.

NRS (not really started) is the Zebrafish orthologue of Drosophila Spinl. The Zebrafish mutant was found in a developmental genetic screen. Mutant embryos accumulate an opaque substance in the yolk and die early in development without any further morphologic abnormalities (Young, Marty, Nakano, Wang, Yamamoto, Lin, and Allende 2002).

No information is available on the actual in vivo activity of mammalian Spinl1 proteins and on the physiological consequences of mutations in the Spinl1 gene and protein in mammals.

### SUMMARY OF THE INVENTION

The invention described herein demonstrates for the first time that Spinl1 is required for the maintenance of a normal metabolism, in particular the fat metabolism. The invention therefore opens novel opportunities for the treatment of diseases associated with an alteration in fat metabolism, e.g., obesity; obesity and diabetes, particularly type II diabetes; or diabetes, particularly type II diabetes, by the modulation of Spinl1 activity.

Furthermore the invention demonstrates for the first time that Spinl1 affects insulin sensitivity. The invention therefore opens novel opportunities in the treatment of diseases associated with an altered insulin level or an alteration in insulin sensitivity, these diseases again including obesity; obesity and diabetes, particularly type II diabetes; and diabetes, particularly type II diabetes. Other diseases to be mentioned in this regard include chronic kidney disease; coronary atherosclerosis; anorexia nervosa; rheumatoid arthritis; osteoarthritis; osteoporosis; gastrointestinal diseases, in particular gastric disease, peptic ulcer, intestinal bowel disease, in particular Crohn's disease or ulcerative colitis; cardiovascular diseases, in particular cardiac hypertrophy; obstructive sleep apnea; maculopathy, in particular age-related maculopathy (ARM) or degeneration (ARMD); and prostate cancer.

The invention provides methods for screening of agents capable of functioning as insulin sensitizers or desensitizers, or agents that are capable of modulating insulin activity, or the signal transduction of the insulin receptor or events further downstream in the insulin signal transduction cascade.

Additionally, the invention demonstrates for the first time that Spinl1 affects leptin sensitivity. The invention therefore opens novel opportunities in the treatment of diseases associated with an altered leptin level or an alteration in leptin sensitivity. In addition to the above-mentioned diseases, such diseases are, e.g., chronic kidney disease; coronary atherosclerosis; anorexia nervosa; rheumatoid arthritis; osteoarthritis; osteoporosis; gastrointestinal diseases, in particular gastric disease, peptic ulcer, intestinal bowel disease, in particular Crohn's disease or ulcerative colitis; cardiac hypertrophy; abstructive sleep apnea; maculopathy, in particular age-related maculopathy (ARM) or degeneration (ARMD); and prostate cancer.

The invention furthermore provides methods for screening of agents capable to functioning as leptin sensitizers or desensitizers or agents that are capable of modulating leptin activity.

The present invention provides inter alia mutant Spinl1 proteins having at least 63%, 65%, 70%, 75%, 80%, 85%, 90%, 95%, 98%, or 990% amino acid identity compared to the mouse Spinl1 or the human Spinl1 protein according to SEQ ID NO:3 and SEQ ID NO:7, or an isolated fragment of such protein comprising at least 6, 7, 8, 9, 10, 15, 20, 25, 30, 35, 40, 50, 60, 70, 80, 90, 100, 150, 200, 250, 300, 350, 400, 450, 460, 470, 480, 490, 500, 510, 520, 521, 522, 523, 524, 525, 526, or 527 contiguous amino acids having said percentages of amino acid identity compared to the corresponding amino acids in SEQ ID NO:3 and SEQ ID NO:7, wherein said protein or fragment of such protein comprises an amino acid or an amino acid sequence which corresponds to a mutation in the mouse Spinl1 protein as defined above which, if encoded by the mouse Spinl1 gene and present in the genome of all or essentially all cells of a mouse in a homozygous manner, results in a phenotype associated with an alteration in fat metabolism compared to the corresponding wild-type animal. In a particularly preferred embodiment, the above proteins or fragments have at least 93% amino acid identity compared to the mouse Spinl1 or the human Spinl1 protein according to SEQ ID NO:3 and SEQ ID NO:7. Also preferred are those of the above proteins or fragments having at least 95% amino acid identity compared to the mouse Spinl1 or the human Spinl1 protein defined above. Again particularly preferred are those of the above proteins or fragments having at least 98% amino acid identity compared to the mouse Spinl1 or the human Spinl1 protein defined above. Furthermore particularly preferred are those of the above proteins or fragments having at least 99% amino acid identity compared to the mouse Spinl1 or the human Spinl1 protein defined above. The latter embodiments are similarly particularly preferred in connection with the proteins and fragments thereof described and claimed herein wherein the protein or protein fragment comprises an amino acid or an amino acid sequence which corresponds to a mutation in the mouse Spinl1 protein as defined above which, if encoded by the mouse Spinl1 gene and present in the genome of all or essentially all cells of a mouse in a homozygous manner, results in a phenotype associated with an alteration in serum leptin level and/or an alteration in leptin sensitivity compared to the corresponding wild-type animal; or with the proteins and fragments thereof described and claimed herein wherein the protein or protein fragment comprises an amino acid or an amino acid sequence which corresponds to a mutation in the mouse Spinl1 protein as defined above which, if encoded by the mouse Spinl1 gene and present in the genome of all or essentially all cells of a mouse in a homozygous manner, results in a phenotype associated with an alteration in plasma insulin level and/or an alteration in insulin sensitivity compared to the corresponding wild-type animal.

The invention further provides orthologues of the above defined mutant Spinl1 proteins having 63%, 65%, 70%, 75%, 80%, 85%, 90%, 95%, 98%, or 99% amino acid identity compared to the mouse Spinl1 or the human Spinl1 protein according to SEQ ID NO:3 or SEQ ID NO:7. In a specific embodiment, the mutant Spinl1 protein has an amino acid substitution of the tyrosine at position 108 by a histidine. In another specific embodiment, the mutant Spinl1 protein has an amino acid sequence as depicted in SEQ ID NO:4 and SEQ ID NO:8. In a further specific embodiment, the mutant Spinl1 protein as defined above, is a fusion protein fused to a protein unrelated to the mouse or the human Spinl1 protein according to SEQ ID NO:3 and SEQ ID NO:7, respectively, e.g. glutathione-S-transferase, an immunoglobulin peptide, a polyhistidine peptide, a FLAG tag, or streptavidin.

The invention furthermore relates to nucleic acids encoding the mutant Spinl1 proteins as defined herein or an isolated nucleic acid, which is complementary thereto. In specific embodiments, the nucleic acid has a sequence as depicted in SEQ ID NO:2 and SEQ ID NO:6.

In one embodiment, the invention relates to an episomal element, a genome, or a vector comprising a nucleic acid encoding a mutant Spinl1 protein, a fragment thereof or a Spinl1 fusion protein as well as to a host cell, transfected with said episomal element, genome, or vector, and methods of producing a mutant Spinl1 protein by culturing said host cells.

The invention also provides an antisense nucleic acid comprising a nucleotide sequence which is complementary to a part of an mRNA encoding a mutant Spinl1 protein as defined in the present invention, or encoding an orthologue thereof, or encoding a protein which affects the expression or activity of the mouse or human Spinl1 protein.

The invention further provides an antisense nucleic acid comprising a nucleotide sequence which is complementary to a part of an mRNA encoding the mouse Spinl1 or the human Spinl1 protein according to SEQ ID NO:3 and SEQ ID NO:7, respectively, or an orthologue thereof, or encoding a protein which affects the expression or activity of the mouse Spinl1 or the human Spinl1 protein according to SEQ ID NO:3 and SEQ ID NO:7, respectively, or an orthologue thereof.

The invention furthermore provides short interfering RNA molecules (Elbashir, Martinez, Patkaniowska, Lendeckel, and Tuschl 2001a) comprising a double stranded nucleotide sequence wherein one strand is complementary to an at least 19, 20, 21, 22, 23, 24, or 25 nucleotide long segment of an mRNA encoding a mutant Spinl1 protein as defined herein, or encoding an orthologue thereof, or encoding a protein which affects the expression or activity of the mouse or human Spinl1 proteins.

The invention furthermore provides short interfering RNA molecules (Elbashir, Martinez, Patkaniowska, Lendeckel, and Tuschl 2001a) comprising a double stranded nucleotide sequence wherein one strand is complementary to an at least 19, 20, 21, 22, 23, 24, or 25 nucleotide long segment of an mRNA encoding the mouse Spinl1 or the human Spinl1 protein according to SEQ ID NO:3 and SEQ ID NO:7, respectively, or an orthologue thereof, or encoding a protein which affects the expression or activity of the mouse Spinl1 or the human Spinl1 protein according to SEQ ID NO:3 and SEQ ID NO:7, respectively, or an orthologue thereof.

The invention further provides an antibody specifically recognizing an epitope in a mutant Spinl1 protein as defined herein, wherein said epitope comprises the amino acid or the amino acid sequence in said protein which corresponds to the mutation in the mutant Spinl1 protein of the invention.

This invention further provides a non-human vertebrate animal comprising in the genome of at least some of its cells an allele of a gene encoding a protein having at least 63%₀, 65%, 70%, 75%, 80%, 85%, 90%, 95%, 98%, or 99% amino acid identity compared to the mouse Spinl1 or the human Spinl1 protein according to SEQ ID NO:3 and SEQ ID NO:7, respectively, said allele comprising a mutation which, if present in the genome of all or essentially all cells of said animal in a homozygous manner, results in a phenotype associated with an alteration in fat metabolism compared to the corresponding wild-type animal. In a particularly preferred embodiment, said gene comprised as an allele in the genome of said cells encodes a protein having at least 90% amino acid identity compared to the mouse Spinl1 or the human Spinl1 protein according to SEQ ID NO:3 and SEQ ID NO:7. Also preferred are those embodiments wherein said gene encodes a protein having at least 93% amino acid identity compared to the mouse Spinl1 or the human Spinl1 protein as defined above. Again particularly preferred are those embodiments of the non-human vertebrate animal of the invention wherein said gene encodes a protein having at least 95% amino acid identity compared to the mouse Spinl1 or the human Spinl1 protein as defined above. Additionally preferred are those embodiments wherein said gene encodes a protein having at least 98% amino acid identity compared to the mouse Spinl1 or the human Spinl1 protein as defined above. Finally, particularly preferred are also embodiments wherein said gene encodes a protein having at least 99% amino acid identity compared to the mouse Spinl1 or the human Spinl1 protein as defined above. The latter embodiments are similarly particularly preferred in connection with the non-human vertebrate animals described and claimed herein wherein said allele comprises a mutation which, if present in the genome of all or essentially all cells of these animals in a homozygous manner, results in a phenotype associated with an alteration in serum leptin level and/or an alteration in leptin sensitivity compared to the corresponding wild-type animal; or with the non-human vertebrate animals described and claimed herein wherein said allele comprises a mutation which, if present in the genome of all or essentially all cells of these animals in a homozygous manner, results in a phenotype associated with an alteration in plasma insulin level and/or an alteration in insulin sensitivity compared to the corresponding wild-type animal.

In one embodiment, the mutation causes a loss of function phenotype. Alternatively, the mutation may cause a gain of function phenotype. In a particularly preferred embodiment, the genome of some or all cells of the non-human vertebrate animal comprises an allele of a gene encoding a mutant Spinl1 protein, particularly a mutant Spinl1 protein with the amino acid tyrosine at position 108 replaced by histidine, e.g. a Spinl1 protein as encoded by SEQ ID NO:4 or SEQ ID NO:8.

In one embodiment, the invention provides a non-human vertebrate animal, wherein the phenotype associated with an alteration in fat metabolism is characterized by an alteration in fat storage, particularly a reduction in fat storage. The alteration may be further characterized by elevated serum levels of components of the cholesterol metabolism and liver enzymes as well as a reduction of lactate and/or by an absence of intracellular fat vacuoles and a size-reduction of white adipocytes. The alteration may furthermore be characterized by a plasma reduction of insulin, or a serum reduction of leptin, or a plasma reduction of glucose. Alternatively, or in addition, the alteration may result in a thriving deficit, particularly a reduction in body weight and/or a reduction in body length, optionally associated with hypoglycemia. The alteration may be further characterized by a reduced plasma insulin level, and/or increased insulin sensitivity.

The present invention also relates to the use of the non-human vertebrate animal for the identification of a protein or nucleic acid diagnostic marker for an alteration in fat metabolism, or as an animal model for studying the molecular mechanisms of, or physiological processes associated with an alteration in fat metabolism; or for the identification and testing of an agent useful in the prevention, amelioration, or treatment of a medical condition associated with an alteration in fat metabolism.

In one embodiment, the non-human vertebrate animal model is used for studying the molecular mechanisms of, or physiological processes associated with, or medical condition associated with, or affected by, reduced activity or undesirable activity of endogenous Spinl1, or reduced expression, reduced production or undesirable production of endogenous Spinl1; or for the identification and testing of an agent useful in the prevention, amelioration, or treatment of these conditions.

In one embodiment, the non-human vertebrate animal model is used for studying or identifying protein or nucleic acid diagnostic markers for an association of an alteration in fat metabolism with altered Spinl1 activity or for identifying binding partners of the Spinl1 protein or genes or proteins regulated by Spinl1 activity and/or deregulated by altered Spinl1 expression.

The invention further relates a pharmaceutical composition comprising a mutant Spinl1 protein or protein fragment as described herein, a nucleic acid encoding such mutant proteins or protein fragments, or an antibody or an immunoconjugate comprising such an antibody, which antibody is directed against a mutant Spinl1 protein as described herein, or an episomal element or vector as described herein, or an antisense nucleic acid as described herein, or an siRNA as described herein, and a pharmaceutically acceptable carrier.

The invention further relates to a pharmaceutical composition comprising an isolated human Spinl1 protein corresponding to the Spinl1 protein of a human subject known not to have a medical condition associated with an alteration in fat metabolism, e.g. obesity; obesity and diabetes, particularly type II diabetes; or diabetes, particularly type II diabetes. Preferably the isolated protein is a protein according to SEQ ID NO:7, or an allelic variant thereof, or a fragment of such protein or allelic variant which is effective in treating said medical condition; or a fusion protein, wherein said Spinl1 protein, said allelic variant, or said fragment of said Spinl1 protein is fused to another protein unrelated to the mouse Spinl1 or the human Spinl1 protein according to SEQ ID NO:3 and SEQ ID NO:7, respectively. The pharmaceutical composition may also comprise an orthologue protein having at least 63%, 65%, 70%, 75%, 80%, 85%, 90%, 95%, 98%, or 99% amino acid identity compared to said Spinl1 protein, allelic variant, or fragment thereof; or an antibody specifically recognizing an epitope comprised within the human Spinl1 protein of a human subject known not to have a medical condition associated with an alteration in fat metabolism, preferably the protein according to SEQ ID NO:7 or an allelic variant thereof; or a (poly)peptide or a small molecule drug, which modulates the activity of the human Spinl1 protein of a human subject known not to have said medical condition, preferably the human Spinl1 protein according to SEQ ID NO:7 or an allelic variant thereof, and a pharmaceutically acceptable carrier.

The invention also relates to a method of preventing, treating, or ameliorating a medical condition in a human subject associated with an alteration in fat metabolism, e.g. obesity; obesity and diabetes, particularly type II diabetes; or diabetes, particularly type II diabetes; said method comprising administering to said human subject a pharmaceutical composition comprising an agent capable of modulating Spinl1 activity in said human subject. In a preferred embodiment, the pharmaceutical composition administered is one as further defined in the present application.

The invention furthermore relates to the mutant or non-mutant Spinl1 protein or protein fragment thereof, the nucleic acid, the antibody or immunoconjugate, the episomal element or vector, the antisense nucleic acid, or siRNA as defined herein for use as a medicament or use for the preparation of a pharmaceutical for preventing, treating, or ameliorating a medical condition in a human subject associated with an alteration in fat metabolism, e.g., obesity; obesity and diabetes, particularly type II diabetes; or diabetes, particularly type II diabetes.

One aspect of the invention relates to a method of gene therapy comprising delivering to cells in a human subject suffering from or known to be at risk of developing a condition associated with an alteration in fat metabolism a DNA construct comprising a sequence of an allele of the Spinl1 gene encoding the human Spinl1 protein of a human subject known not to have a medical condition associated with an alteration in fat metabolism; or a DNA construct comprising a sequence encoding the human Spinl1 protein of a human subject known not to have a medical condition associated with an alteration in fat metabolism; or a DNA construct comprising a sequence encoding the human Spinl1 protein of a human subject known not to have a medical condition associated with an alteration in fat metabolism; or a DNA construct comprising a sequence encoding an antisense nucleic acid as described herein, or a sequence encoding an siRNA as described herein. Another aspect is the use of said DNA construct for the preparation of a pharmaceutical for the treatment of a medical condition associated with an alteration in fat metabolism, or the prevention of said medical condition in a human subject known not to be at risk of developing such a condition.

The invention furthermore relates to a method of identifying a binding partner of the Spinl1 protein, the method comprising contacting a candidate binding partner with a wild-type mammalian Spinl1 protein, preferably the mouse Spinl1 protein or the human Spinl1 protein according to SEQ ID NO:3 and SEQ ID NO:7, respectively, under physiological conditions; and determining whether binding of the candidate binding partner to said Spinl1 protein occurred, e.g., by NMR technology.

The invention also relates to a method of identifying an antagonist of the Spinl1 protein, the method comprising culturing mammalian cells in the presence or absence of a wild type mammalian Spinl1 protein, preferably the mouse Spinl1 protein or the human Spinl1 protein according to SEQ ID NO:3 and SEQ ID NO:7, respectively; and determining whether an increase in size of endosomal and/or lysosomal compartments is observed in the presence of said wild type Spinl1 upon addition of a candidate antagonist agent to the cultured cells. In a preferred embodiment, said cells show a reduced or no expression of endogenous Spinl1 protein, or carry a mutation in one or both alleles of their endogenous Spinl1 gene so that the allele is no longer capable of being expressed or that it encodes a mutant protein as described herein which is characterized by a loss of function phenotype.

### BRIEF DESCRIPTION OF THE FIGURES

FIG. 1 depicts differences in nine out of sixteen blood parameters determined in blood samples of homozygous affected mice, when compared to wild type mice. Elevated levels were detected for alkaline phosphatase (ALP), glutamic-oxaloacetic transaminase (GOT), glutamate pyruvate transaminase (Fallner and Moss 1988), lactate dehydrogenase (LDH), cholinesterase (CHE), cholesterol (CHOL), high density liprotein (HDL) and low density lipoprotein (Cohen, Miyazaki, Socci, Hagge-Greenberg, Liedtke, Soukas, Sharma, Hudgins, Ntambi, and Friedman 2002). Reduced level was detected for lactate (LACT) (see Example 2). Units are indicated in brackets. Abnormal levels of blood parameters are indicative for disturbed liver function. Animals were analyzed at the age of 93 days.

FIG. 2 depicts differences in body weight of homozygous affected (aff) female (a) and male (b) mice, compared to wild type (control, co) mice of an age between 35 and 91 days. Body weight (BW) is indicated in gram (g). Numbers of affected and wild type mice are indicated (n=).

FIG. 3 depicts in (a) an allometric representation of body length versus body weight of homozygous affected males and in (b) an allometric representation of fat mass versus body weight of homozygous affected females and males. Body length (in mm) and fat mass (in gram, g) are reduced in affected mice, with fat mass being more reduced than body length Animals were at the age of 132 to 148 days.

FIG. 4a depicts cross sections from homozygous affected (homozygous) and wild type (wild type) kidneys, after staining with Haematoxylin & Eosin (H&E). The morphology is largely undisturbed in homozygous affected mice, except for the absence of interstitial fat vacuoles. White arrows in the wild type kidney section point at fat vacuoles. Magnification is ×40 lens.

FIG. 4b depicts areas of perirenal fat pad from the cross sections shown in FIG. 4a Brown adipose tissue (arrowhead) is present in both, homozygous affected (homozygous) and wild type (wild type) mice. A darker appearence of brown adipose tissue in the section of the homozygous animal is due to a reduced content of lipid droplets. In the homozygous kidney white adipocytes (arrow) with reduction in size are detected, compared to wild type. Magnification is ×20 lens.

FIG. 5a depicts cross sections from homozygous affected (homozygous) and wild type (wild type) livers, after staining with Haematoxylin & Eosin (H&E). In the homozygous section, the distribution of cytoplasmic staining is towards the sinusoidal pole compared to a homogenous distribution observed in the wild type section. Arrows in the homozygous section point to councilman bodies, representing apoptotic hepatocytes. Magnification is ×100 lens.

FIG. 5b depicts electron microscopic pictures of homozygous affected (homozygous) and wild type (wild type) livers, prepared by the transmission electron microscopy method. In the homozygous section, lamellar electron dense material (arrow) and a reduced number of fat vacuoles (arrowhead) is detected, compared to the situation observed in the wild type section.

FIG. 6 depicts a chart correlating food consumption (food in gram per gram body weight, food [g/g BW]) to body weight (BW[g]), comparing homozygous affected (hom), heterozygous affected (het) and wild type (wt) mice. Though body weight is reduced, food consumption is not significantly different between homozygous affected, heterozygous affected and wild type mice.

FIG. 7 depicts data from macro-mapping of the Spinl1 mutation. Genome-wide linkage analysis in affected mice was performed using in-house SNP panels and pyrosequencing technology. The value for a heterozygous situation at a certain locus is 1. Spinl1 affected mice are homozygous C3H for marker 55-Sox6 on chromosome 7.

FIG. 8 depicts a haplotype scheme confirming the initial mapping on a single mouse level. Haplotype analysis was performed on affected mice using SNP or microsatellite markers located in the critical region on chromosome 7. The candidate region mapping was refined by analyzing mice carrying chromosomal break points in the respective region. This analysis narrowed the location of the mutation to an interval of approximately 1.39 Mbp between the microsatellite marker D71 ng57 and SNP marker Q9D1C0-9-10.

FIG. 9 depicts a mouse multi-tissue Northern blot hybridized with a mouse Spinl1 DNA probe. Agarose gels containing ethidiumbromide were photographed at an UV transilluminator visualizing the 28S- and 18S-rRNA and the RNA size marker bands (FIG. 9A). After hybridization of a radiolabeled 1032 bp probe specific for mouse Spinl1 the RNA filter was exposed to an X-ray film. Spinl1 specific signals were detected in several tissues (FIG. 9B).

FIG. 10 depicts an amino acid sequence alignment of mouse and human Spinl1 proteins. Black boxes indicate identical amino acids, grey boxes indicate similar amino acids.

FIG. 11 depicts an amino acid sequence alignment of mouse, human, and rat Spinl1 proteins, indicating evolutionary highly conserved amino acid residues. Black boxes indicate identical amino acids, grey boxes indicate similar amino acids. The conserved amino acids (i.e., identical or similar) are listed in Table 1.

FIG. 12 depicts an amino acid sequence alignment of mouse, human, rat, and zebrafish Spinl1 proteins, indicating evolutionary highly conserved amino acid residues. Black boxes indicate identical amino acids, grey boxes indicate similar amino acids. The conserved amino acids (i.e., identical or similar) are listed in Table 2.

FIG. 13 depicts an amino acid sequence alignment of mouse, human, rat, zebrafish, and fugu Spinl1 proteins, indicating evolutionary highly conserved amino acid residues. Black boxes indicate identical amino acids, grey boxes indicate similar amino acids. The conserved amino acids (i.e., identical or similar) are listed in Table 3.

FIG. 14 depicts body weight data of homozygous Spinl1 affected mice and of wild type mice measured during a time-course of 17 weeks. Mice were fed with normal food diet (chow) or with a high fat diet (HFD).

FIG. 15 depicts plasma glucose levels measured in homozygous Spinl1 affected mice and wild type mice fed with either normal food diet (chow) or high fat diet (HFD), respectively. Glucose levels were determined after overnight starving (starve) and after refeeding (refed) with either chow or HFD.

FIG. 16 depicts serum leptin levels measured at homozygous Spinl1 affected mice and wild type mice fed with either normal food diet (chow) or high fat diet (HFD), respectively. Leptin levels were determined after overnight starving (starve) and after refeeding (refed) with either chow or HFD (FIG. 16A). The average daily food consumption of homozygous Spinl1 affected mice and of wild type mice was measured during normal food diet (chow) and high fat diet (HFD), respectively (FIG. 16B).

FIG. 17 depicts the body composition of wild type mice (wt), homozygous Spinl1 mice (chg), homozygous obese mice (ob; lacking the leptin gene), and of Spinl1/ob double-homozygous mice (chg/ob). The amounts of lean, fat, and the total weight [weight]) were determined.

FIG. 18 depicts Western blotting (WB) data, detecting phosphorylated STAT3 (pStat3) and unphosphorylated STAT3 (Stat3) in liver of homozygous Spinl1 mice after administration of leptin for 15, 30, and 60 minutes, respectively. In a control experiment at wild type mice, detection of phosphorylated and unphosphorylated STAT3 was performed at the time of leptin administration start (0) and after adminisration of leptin for 15, 30, and 60 minutes, respectively. STAT3 was detected by immunoprecipitation (IP) with an anti-STAT3 antibody (anti-Stat3). Selective detection of phosphorylated STAT3 was performed by applying an antibody generated against phosphorylated Tyrosin 705 of STAT3 (WB: anti-pTyr705 Stat3). Overall amounts of immunoprecipitated STAT3 was determined by stripping the protein membrane and applying anti-Stat3 (WB: anti-Stat3 after stripping). Phosphorylated STAT3 is undetected in 3T3-L1 cells (3T3-L1), as seen in FIG. 18A. FIG. 18B depicts Western blotting (WB) data, detecting phosphorylated AKT (pAkt) and unphosphorylated AKT (Akt) in liver of homozygous Spinl1 mice after administration of leptin for 15, 30, and 60 minutes, respectively. The state of AKT phosphorylation was also determined at the time of leptin administration start (0). In a control experiment with wild type mice, detection of phosphorylated and unphosphorylated AKT was performed at the time of leptin administration start (0) and after administration of leptin for 15, 30, and 60 minutes, respectively. AKT was detected by immunoprecipitation (IP) with an anti-AKT antibody (anti-Akt). Selective detection of phosphorylated AKT was performed by applying an antibody generated against phosphorylated Serin 473 of AKT (WB: anti-pSer473 Akt). Overall amounts of immunoprecipitated AKT was determined by stripping the protein membrane and applying anti-Akt (WB: anti-Akt after stripping).

FIG. 19 depicts Western blotting (WB) data, detecting phosphorylated STAT3 (pStat3) and unphosphorylated STAT3 (Stat3) in muscle of homozygous Spinl1 mice after administration of leptin for 15, 30, and 60 minutes, respectively. In a control experiment at wild type mice, detection of phosphorylated and unphosphorylated STAT3 was performed at the time of leptin administration start (0) and after administration of leptin for 15, 30, and 60 minutes, respectively. STAT3 was detected by immunoprecipitation (IP) with an anti-STAT3 antibody (anti-Stat3). Selective detection of phosphorylated STAT3 was performed by applying an antibody generated against phosphorylated Tyrosin 705 of STAT3 (WB: anti-pTyr705 Stat3). Overall amounts of immunoprecipitated STAT3 was determined by stripping the protein membrane and applying anti-Stat3 (WB: anti-Stat3 after stripping). Phosphorylated STAT3 is detected in 3T3-L1 cells (3T3-L1) at low level, as seen in FIG. 19A. FIG. 19B depicts Western blotting (WB) data, detecting phosphorylated AKT (pAkt) and unphosphorylated AKT (Akt) in muscle of homozygous Spinl1 mice after administration of leptin for 15, 30, and 60 minutes, respectively. The state of AKT phosphorylation was also determined at the time of leptin administration start (0). In a control experiment with wild type mice, detection of phosphorylated and unphosphorylated AKT was performed at the time of leptin administration start (0) and after administration of leptin for 15, 30, and 60 minutes, respectively. AKT was detected by immunoprecipitation (IP) with an anti-AKT antibody (anti-Akt). Selective detection of phosphorylated AKT was performed by applying an antibody generated against phosphorylated Serin 473 of AKT (WB: anti-pSer473 Akt). Overall amounts of immunoprecipitated AKT was determined by stripping the protein membrane and applying anti-Akt (WB: anti-Akt after stripping).

FIG. 20 depicts plasma insulin levels measured in respect of homozygous Spinl1 affected mice and wild type mice fed with either normal food diet (chow), or high fat diet (HFD), respectively. Insulin levels were determined after overnight starving (starv) and after refeeding (refed) with either chow or HFD. Plasma insulin levels are depicted in picogram per milliliter (pg/ml).

FIG. 21 depicts data of an Insulin Resistance Test (IRT), performed with homozygous Spinl1 affected mice, and wild type mice as control. Blood samples of mice are taken before insulin injection (IRT time [min] 0), and 15 min, 30 min, 60 min, 90 min, 120 min, 150 min, 180 min, and 240 min, respectively, after insulin injection (IRT time [min] 15, 30, 60, 90, 120, 150, 180, 240). The glucose levels measured are depicted as percentage relative to the glucose level at IRT time [min] 0.

### List of Tables Provided

Table 1 summarizes conserved amino acid residues in Spinl1 proteins when comparing mouse, rat and human Spinl1. Conserved residues are numbered as human Spinl1 amino acid positions.

Table 2 summarizes conserved amino acid residues in Spinl1 proteins when comparing mouse, rat, human, and zebrafish Spinl1. Conserved residues are numbered as human Spinl1 amino acid positions.

Table 3 summarizes conserved amino acid residues in Spinl1 proteins when comparing mouse, rat, human, zebrafish, and fugu Spinl1. Conserved residues are numbered as human Spinl1 amino acid positions.

### DETAILED DESCRIPTION OF THE INVENTION

The various aspects and utilities of the present invention will be apparent from the following detailed description.

Nuclei Acids

The present invention provides nucleic acid sequences encoding the Spinl1 proteins as described in more detail above and below, for example murine and human Spinl1 mutated in accordance with the present invention. In a preferred embodiment, this invention provides a mutant nucleic acid sequence encoding mouse and human Spinl 1 protein (SEQ ID NO:2 and SEQ ID NO:6). Mutant mouse and human Spinl1 encoding nucleic acids or genes, can be made, for example, by altering codon 108 of the wild type human Spinl1 gene, such that codon 108 no longer encodes tyrosine. The construction of a gene with a 108^{th} codon that does not encode tyrosine can be achieved by methods well known in the art.

Tyrosine is encoded by TAT or TAC. A codon that does not encode tyrosine may be, for example, a codon that encodes Phe (TTT, TTC); Leu (TTA, TTG, CTT, CTC, CTA, CTG); Ile (ATT, ATC, ATA); Met (ATG); Asp (GAC, GAT); Ser (TCT, TCC, TCA, TCG), Val (GTT, GTC, GTA and GTG); Pro (CCT, CCC, CCA, CCG); Thr (ACT, ACC, ACA, ACG), Ala (GCT, GCC, GCA, GCG); His (CAT, CAC), Gln (CAA, CAG); Asn (AAT, AAC); Lys (AAA, AAG); Glu (GAA, GAG); Cys (TGT, TGC); Trp (TGG); Arg (CGT, CGC, CGA, CGG, AGA, AGG); Ser (AGT, AGC); Gly (GGT, GGC, GGA, GGG) or one of the stop codons (TAA, TAG, TGA). Again, methods for the introduction of site-specific nucleic acid mutations are well known.

The nucleic acid sequences encoding the mutant Spinl1 proteins, and fragments thereof, of the invention may exist alone or in combination with other nucleic acid sequences, for example, within episomal elements, genomes, or vector molecules, such as plasmids, including expression or cloning vectors.

The term “nucleic acid sequence” as used herein refers to any contiguous sequence series of nucleotide bases, i.e., a polynucleotide, and is preferably a ribonucleic acid (RNA) or deoxy-ribonucleic acid (DNA). Preferably the nucleic acid sequence is cDNA. It may, however, also be, for example, a peptide nucleic acid (PNA).

An “isolated” nucleic acid molecule, as referred to herein, is one, which is separated from other nucleic acid molecules ordinarily present in the natural source of the nucleic acid. Preferably, an “isolated” nucleic acid is free of sequences, which naturally flank the nucleic acid (i.e., sequences located at the 5′- and 3′-termini of the nucleic acid) in the genomic DNA of the organism that is the natural (wild type) source of the DNA.

The term “mutant” or “modified” as used herein in connection with the Spinl1 protein sequences and nucleic acid sequences relating thereto refers to an alteration in the sequence compared to the corresponding wild type Spinl1.

Spinl1 gene molecules can be isolated using standard hybridization and cloning techniques, as described, for instance, in Sambrook et al. (eds.), MOLECULAR CLONING: A LABORATORY MANUAL (2^{nd} Ed.), Cold Spring Harbor Laboratory Press, Cold Spring Harbor, N.Y., 1989; and Ausubel et al. (eds.), CURRENT PROTOCOLS IN MOLECULAR BIOLOGY, John Wiley & Sons, New York, N.Y., 1993.

A nucleic acid of the invention can be amplified using cDNA, mRNA or, alternatively, genomic DNA, as a template and appropriate oligonucleotide primers according to standard polymerase chain reaction (PCR) amplification techniques. The nucleic acid so amplified can be cloned into an appropriate vector and characterized by DNA sequence analysis. Furthermore, oligonucleotides corresponding to Spinl1 nucleotide sequences according to the invention can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction or to be used for hybridization under physiological or more stringent conditions. A short oligonucleotide sequence may be based on, or designed from, a genomic or cDNA sequence and is used to amplify, confirm, inhibit, or reveal the presence of an identical, similar or complementary DNA or RNA in a particular cell or tissue. Generally, the term “oligonucleotide” is used to refer to a series of contiguous nucleotides (a polynucleotide) of about 100 nucleotides (nt) or less, e.g., portions of a nucleic acid sequence of about 100 nt, 50 nt, or 20 nt in length, preferably nucleotide sequences of about 15 nt to 30 nt in length The oligonucleotides of the present invention can be used as primers for the polymerase chain reaction, as hybridization probes in blotting experiments, as siRNAs, or as antisense oligonucleotides for inhibiting the expression or function of a Spinl1 encoding nucleic acid.

As used herein, the term “complementary” refers to Watson-Crick or Hoogsteen base pairing between nucleotide units of a nucleic acid molecule, and the term “binding” means the physical or chemical interaction between two polypeptides or compounds or associated polypeptides or compounds or combinations thereof.

A “homologous nucleic acid sequence” or “homologous amino acid sequence,” or variations thereof, refers to sequences characterized by a homology at the nucleotide level or amino acid level, respectively. Homologous nucleotide sequences can include those sequences coding for isoforms of Spinl1 polypeptides. Isoforms can be expressed in different tissues of the same organism as a result of, for example, alternative splicing of RNA. Alternatively, isoforms can be encoded by different genes.

As used herein, the phrase “stringent hybridization conditions” refers to conditions under which a probe, primer or oligonucleotide or any other nucleic acid sequence referred to herein will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3, and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide. Stringent conditions are known to those skilled in the art and can be found in Ausubel et al. (eds.), CURRENT PROTOCOLS IN MOLECULAR BIOLOGY, John Wiley & Sons, N.Y. (1989), 6.3.1-6.3.6.

Preferred stringent hybridization conditions in accordance with the nucleic acids of the present invention, for example the antisense nucleic acids described further below, are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C.

As used herein, for example, in connection with the antisense nucleic acids of the present invention described further below, the phrase “hybridization under physiological conditions” refers to hybridization of a probe, primer or oligonucleotide, or any other nucleic acid sequence to its target sequence under conditions as they are found inside eukaryotic cells either within a multicellular organism or under conditions of cell or tissue culture. Typically, “physiological conditions” are characterized by a temperature of about or exactly 37° C., absence of formamide, and an ionic strength corresponding to physiological buffer with 280 to 300 mOsmol, and a pH value of 7.4.

Vectors and Cells Expressing Spinl1 Protein.

Another aspect of the invention pertains to vectors, preferably expression vectors, containing a nucleic acid encoding a mutant Spinl1 protein, or derivatives, fragments, analogs, homologs or fusion proteins thereof. As used herein, the term “vector” refers to a nucleic acid molecule capable of transporting another nucleic acid to which it has been linked. One type of suitable vector is a “plasmid”, which refers to a circular double stranded circular DNA molecule into which additional DNA segments can be ligated. Another suitable type of vector is a viral vector, wherein additional DNA segments can be ligated into a viral genome or parts thereof. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”.

A host cell of the invention, such as a prokaryotic or eukaryotic host cell in culture, can be used to produce (i.e., express) mutant Spinl1 protein. Accordingly, the invention further provides a method for producing mutant Spinl1 protein using the host cells of the invention. In one embodiment, the method comprises culturing the host cell of the invention (into which a recombinant expression vector encoding mutant Spinl1 protein has been introduced) in a suitable medium such that mutant Spinl1 protein is produced. In another embodiment, the method further comprises isolating mutant Spinl1 protein, i.e., recombinantly produced protein, from the medium or the host cell.

The host cells of the invention can also be used to produce non-human transgenic animals. For example, in one embodiment, a host cell of the invention is a fertilized oocyte or an embryonic stem cell into which Spinl1 protein-coding sequences have been introduced. Such host cells can then be used to create non-human transgenic animals in which exogenous Spinl1 sequences have been introduced into their genome, or animals created by homologous recombination, in which endogenous Spinl1 sequences have been altered.

Proteins and Amino Acids

The present invention also provides mutant Spinl1 amino acid sequences, for example, murine and human mutant Spinl1 amino acid sequences. The wild type murine and human Spinl1 amino acid sequences are shown in SEQ ID NO:3 and SEQ ID NO:7, respectively. A preferred mutant version of the mouse and human Spinl1 amino acid sequence is one wherein tyrosine at position 108 is mutated to a non-tyrosine amino acid.

More generally, the present invention provides a protein having at least 63,%, 65%, at least 70%, at least 75%, at least 80%, at least 85%, at least 90%, at least 95%, at least 98%, or at least 99% amino acid identity compared to the mouse Spinl1 or the human Spinl1 protein according to SEQ ID NO:3 and SEQ ID NO:7, respectively. Also encompassed by the present invention are fragments of such proteins comprising at least 6, at least 7, at least 8, at least 9, at least 10, at least 15, at least 20, at least 25, at least 30, at least 35, at least 40, at least 50, at least 60, at least 70, at least 80, at least 90, at least 100, at least 150, at least 200, at least 250, at least 300, at least 350, at least 400, at least 450, at least 460, at least 470, at least 480, at least 490, at least 500, at least 510, at least 520, at least 521, at least 522, at least 523, a least 524, at least 525, at least 526 or at least 527 contiguous amino acids having the above percentages of amino acid identity compared to the corresponding amino acids in SEQ ID NO:3 and SEQ ID NO:7.

The following definitions apply to any reference to nucleic acid or amino acid sequence identity throughout the present specification:

The term “sequence identity” refers to the degree to which two polynucleotide or polypeptide sequences are identical on a residue-by-residue basis over a particular region of comparison.

The phrases “percent amino acid identity” or “% amino acid identity” refer to the percentage of sequence identity found in a comparison of two or more amino acid or nucleic acid sequences. Percent identity can be readily determined electronically, e.g., by using the MEGALIGN program (DNASTAR, Inc., Madison Wis.). The MEGALIGN program can create alignments between two or more sequences according to different methods, one of them being the clustal method. See, e.g., Higgins and Sharp (Higgins and Sharp 1988). The clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity.

Percent identity can also be readly determined electronically, by using the MultAlin software (Corpet 1988).

A particularly preferred method of determining amino acid identity between two protein sequences for the purposes of the present invention is using the “Blast 2 sequences” (b12seq) algorithm described by Tatusova et al. (Tatusova and Madden 1999). This method produces an alignment of two given sequences using the “BLAST” engine. On-line access of “blasting two sequences” can be gained via the NCBI server at http://www.ncbi.nlm.nih.gov/blast/b12seq/b12.html. The stand-alone executable for blasting two sequences (b12seq) can be retrieved from the NCBI ftp site (ftp://ftp.ncbi.nih.gov/blast/executables). Preferably, the settings of the program blastp used to determine the number and percentage of identical or similar amino acids between two proteins are the following:

| | |
|---|---|
| Program: | blastp |
| Matrix: | BLOSUM62 |
| Open gap penalty: | 11 |
| Extension gap penalty: | 1 |
| Gap x_dropoff: | 50 |
| Expect: | 10.0 |
| Word size: | 3 |
| Low-complexity filter: | on |

For the purposes of the present specification, a reference to percent amino acid sequence identity means in a preferred embodiment percent identity as determined in accordance with the blastp program using the above settings.

The comparison of two or more amino acid or nucleic acid sequences to determine sequence identity can be performed between orthologue sequences, preferably between mouse and human, more preferably between mouse, rat, and human sequences. When a position of an amino acid or nucleotide in one orthologue sequence is occupied by the same amino acid or nucleotide in at least a second orthologue sequence, this amino acid or nucleotide is “evolutionary conserved” for the purpose of this invention. The term “evolutionary conserved” also comprises amino acid substitutions, where an amino acid is replaced by another (i.e., different) amino acid that represents a conservative substitution as defined below.

In accordance with the invention described herein, the above protein or protein fragment comprises an amino acid or an amino acid sequence which corresponds to a mutation in the mouse Spinl1 protein according to SEQ ID NO:3 which, if encoded by the mouse Spinl1 gene and present in the genome of all or essentially all cells of a mouse in a homozygous manner, results in a phenotype associated with an alteration in fat metabolism compared to the corresponding wild type animal.

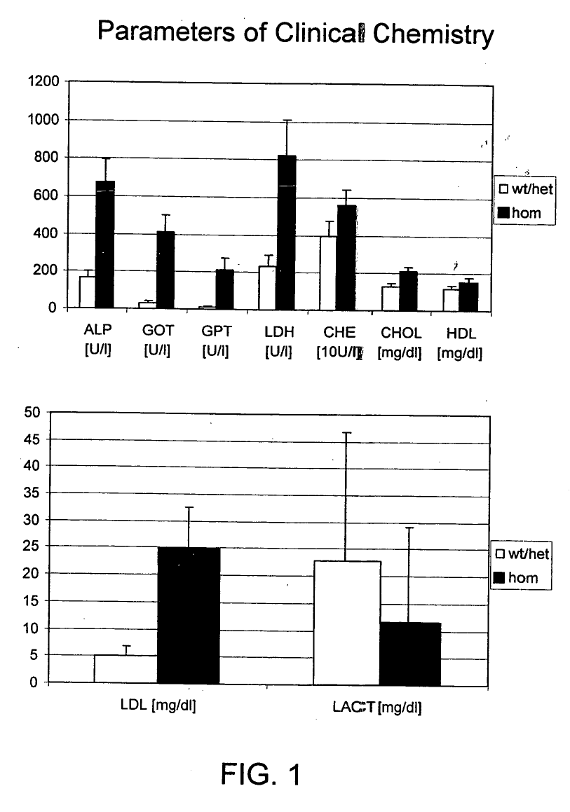

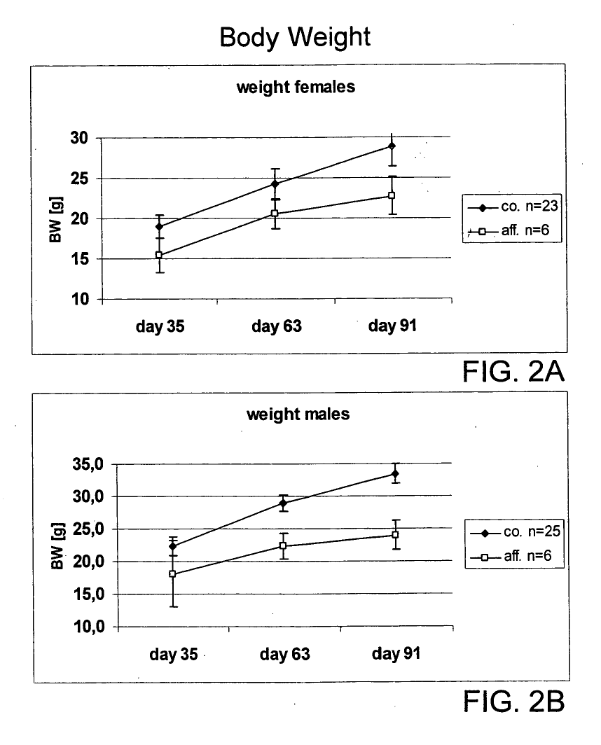

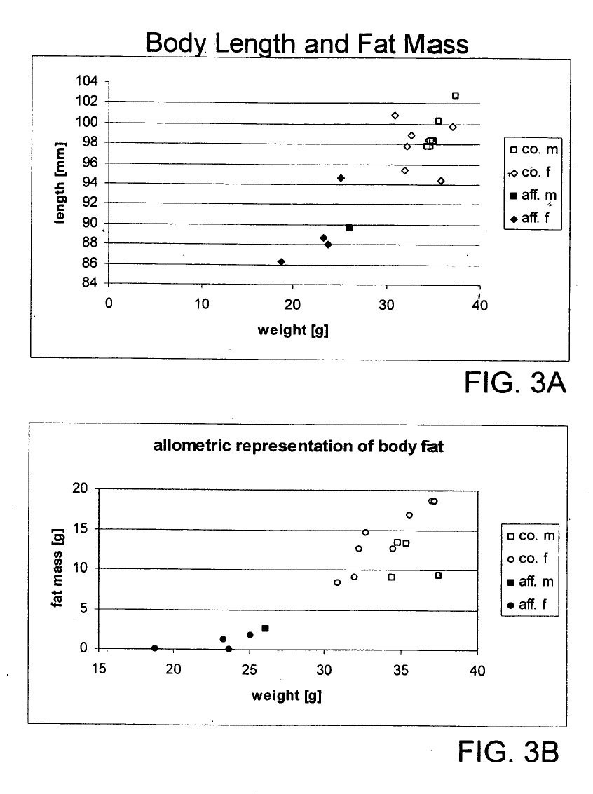

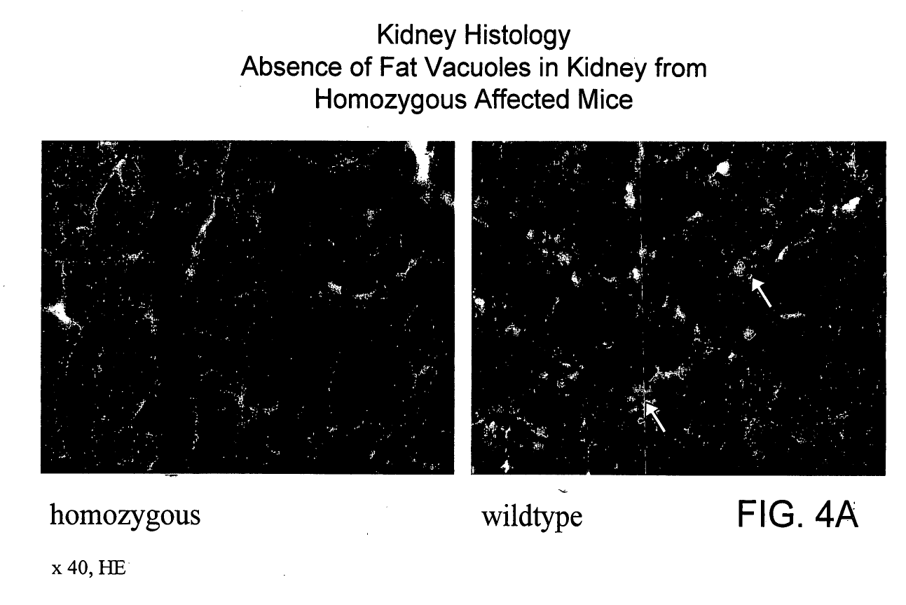

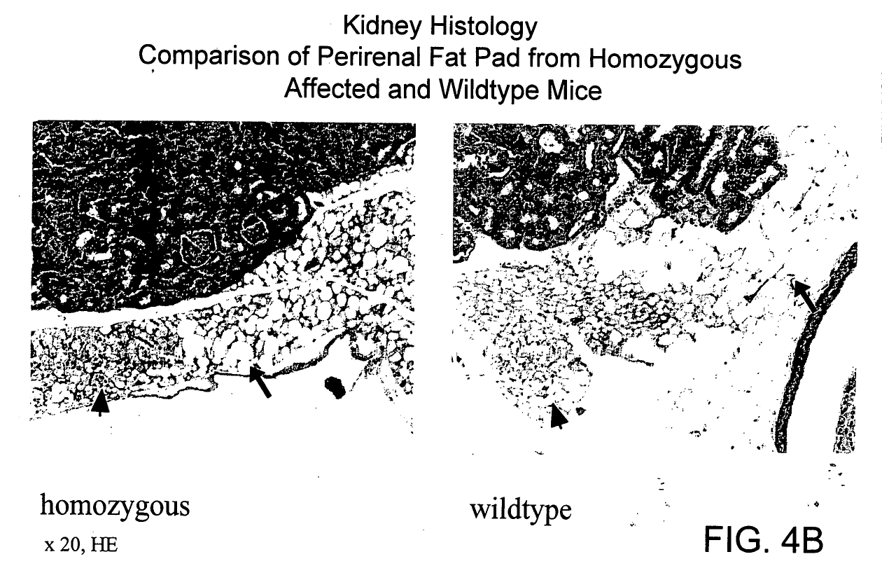

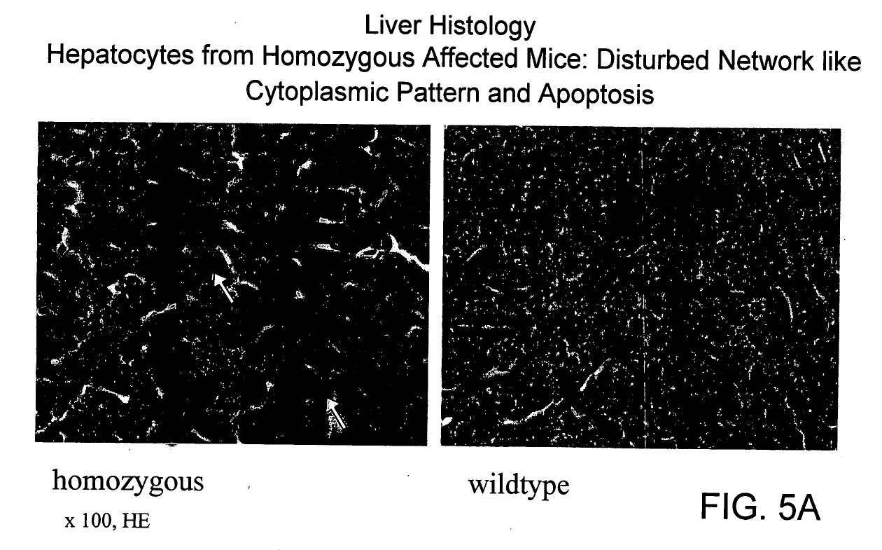

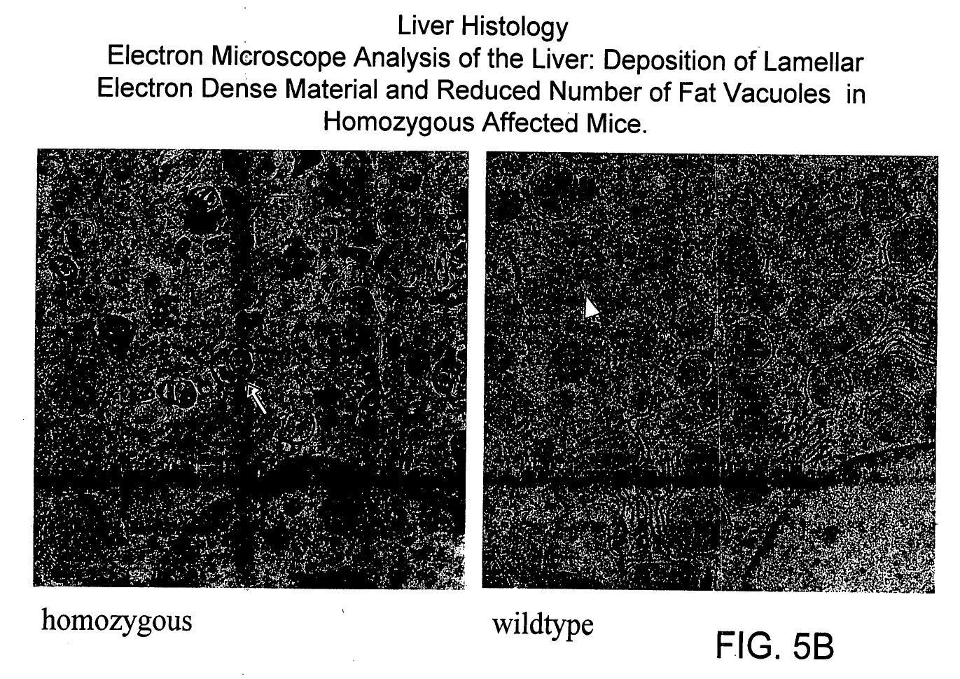

The term “phenotype associated with an alteration in fat metabolism” as referred to throughout the present application may be characterized by an alteration in fat storage, particularly a reduction in fat storage. Alternatively or in addition, the phenotype may be characterized by an alteration in liver function. In respect of the alteration in fat storage, a typical phenotype of a non-human vertebrate animal according to the invention is one characterized by an absence or size-reduction of intracellular fat vacuoles, a size-reduction of white adipocytes (see FIG. 4) and/or an elevated blood serum level for components of the cholesterol metabolism, e.g. cholesterol, cholinesterase, high density lipoprotein, and low density lipoprotein. In respect of the alteration in liver function, a typical phenotype is one characterized by an alteration of liver histology (see FIG. 5) and liver parameters in the serum (see FIG. 1). Specifically, elevated blood serum levels are preferably observed for liver enzymes, such as alkaline phosphatase, glutamic-oxaloacetic transaminase, glutamate pyruvate transaminase, and lactate dehydrogenase. A reduction of serum level was observed for lactate. As to the liver histology, the liver of said animals display no network like cytoplasmic pattern, an increased number of apoptotic hepatocytes, and the accumulation of electron dense material in the hepatocytes (see FIG. 5). Furthermore, a typical phenotype of a non-human vertebrate animal according to the present invention is one wherein the alteration results in a thriving deficit Specifically, said animals display a reduced body weight and body length compared to wild type animals, although food consumption is not significantly reduced in said animals (see FIGS. 2 and 3). Alternatively, the alteration may result in an increase of body weight.

The term “corresponds to” as used in this regard and throughout the present specification means that the mutated allele reflects the mutation in the mouse Spinl1 protein according to SEQ ID NO:3 on the amino acid level. Where the sequences of the allele of the Spinl1 gene flanking the mutation do not encode amino acids identical to those at the corresponding positions in the amino acid sequences of the mouse Spinl1 protein defined above, the skilled artisan will be readily able to align the amino acid sequences encoded by the flanking sequences with the corresponding amino acids of the mouse Spinl1 protein, preferably by using the above-mentioned method of determining amino acid sequence identity, and determine whether a mutation in the mouse Spinl1 protein of the kind mentioned above is reflected by the amino acid sequence encoded by said allele. In case of an amino acid substitution or insertion, the mutation is preferably reflected by the amino acid sequence encoded by the allele in such a way that an identical amino acid or amino acid sequence is found at the corresponding position of the protein encoded by the allele. In case of an amino acid deletion, the mutation is preferably reflected by the amino acid sequence encoded by the allele in such a way that an identical or corresponding amino acid or amino acid sequence is deleted at the corresponding position of the protein encoded by the allele.

In a preferred embodiment, the protein of the invention represents an orthologue of the mouse Spinl1 or the human Spinl1 protein according to SEQ ID NO:3 and SEQ ID NO:7, preferably a vertebrate orthologue, in particular an orthologue wherein said vertebrate is a fish orthologue, in particular Danio rerio or Takifugus rubiens. Alternatively, it may represent a mammalian orthologue, in particular a rat, rabbit, hamster, dog, cat, sheep bovine, or horse orthologue. It may also be a variant of the mouse Spinl1 protein or the human Spinl1 protein according to SEQ ID NO:3 and SEQ ID NO:7, respectively, or of said orthologue, allelic variant or otherwise, wherein certain amino acids or partial amino acid sequences have been replaced, added, or deleted.

Again in a preferred embodiment, the mutation mentioned above results in a deletion or substitution by another amino acid of an amino acid of said mouse Spinl1 protein according to SEQ ID NO:3. Alternatively, the mutation may result in an insertion of additional amino acids not normally present in the amino acid sequence of the mouse Spinl1 protein defined above.

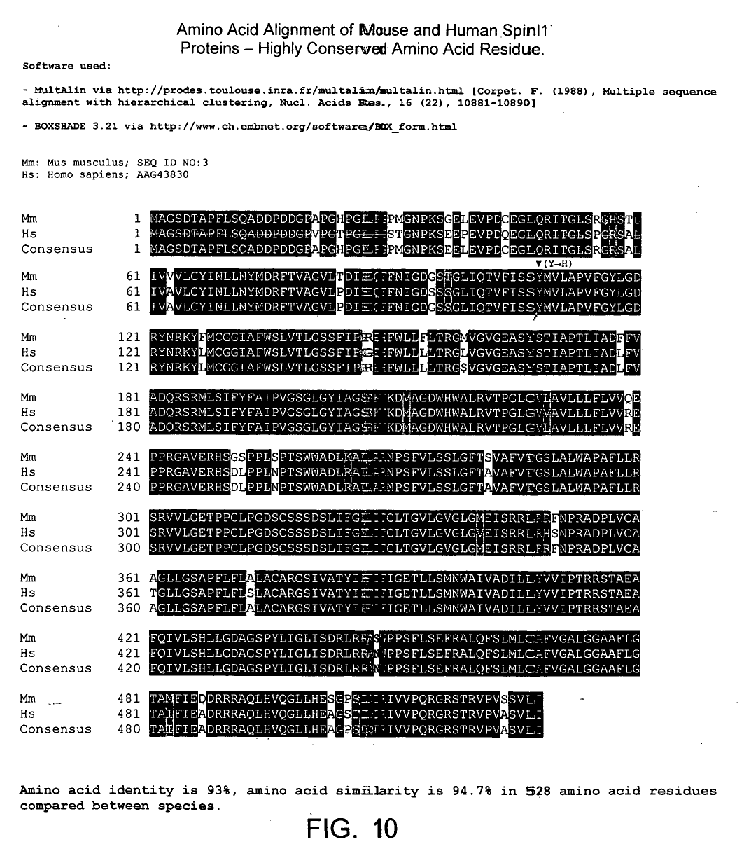

The deletion, substitution, or insertion may furthermore occur in an evolutionary conserved region of said mouse Spinl1 protein. In particular, it may be a substitution of an amino acid, which is identical or similar between mouse and human Spinl1, by another amino acid. Such an amino acid may be a non-naturally occurring or a naturally ocurring amino acid. Preferably, it is a substitution of an amino acid, which is identical or similar between mouse, human, and rat, more preferably between mouse, human, rat and zebrafish, and particularly preferred between mouse, human, rat, zebrafish, and fugu Spinl1 by another amino acid. The skilled artisan will be readily able to determine regions which are generally evolutionary conserved amongst different species on the basis of sequence comparisons such as those shown in FIGS. 10 to 13. The amino acids identical or similar between the species specifically mentioned above will furthermore be readily identifiable by the skilled artisan on the basis of the amino acid sequence comparisons depicted in Tables 1, 2, or 3.

Preferably, the wild type residue of the modified Spinl1 protein is replaced by an amino acid with different size and/or polarity, i.e., a non-conservative amino acid substitution, as defined below.

Also preferred is a Spinl1 mutant protein wherein the residue corresponding to residue 108 of Spinl1 according to SEQ ID NO: 3 or SEQ ID NO:7 is replaced by an amino acid other than a large aromatic amino acid, and preferably is replaced by a basic amino acid, and most preferably by a histidine.

An “isolated” or “purified” polypeptide or protein, or a biologically active fragment thereof as described and claimed herein, is substantially free of cellular material or other contaminating proteins from the cell or tissue source from which the polypeptide or protein is derived, or substantially free from chemical precursors or other chemicals when chemically synthesized. The language “substantially free of cellular material” includes preparations of Spinl1 protein in which the protein is separated from cellular components of the cells from which the protein is isolated or in which it is recombinantly produced.

The invention furthermore encompasses mature mouse Spinl1 or human Spinl1 proteins, or their vertebrate orthologues, e.g., the specific orthologues referred to above, which comprise an amino acid or amino acid sequences corresponding to a mutation as defined herein. As used herein, a “mature” form of a polypeptide or protein may arise from a post-translational modification. Such additional processes include, by way of non-limiting example, proteolytic cleavage, e.g., cleavage of a leader sequence, glycosylation, myristoylation or phosphorylation. In general, a mature polypeptide or protein according to the present invention may result from the operation of one of these processes, or a combination of any of them.

As mentioned above, when for example residue 108 of SEQ ID NO:3 or SEQ ID NO: 7 is replaced by an amino acid with different size and/or polarity (excluding the wild type residue at this position), this is termed a non-conservative amino acid substitution. Non-conservative substitutions are defined as exchanges of an amino acid by another amino acid listed in a different group of the five standard amino acid groups shown below:
1. small aliphatic, nonpolar or slightly polar residues: Ala, Val, Ser, Thr, (Pro), (Gly);
2. negatively charged residues and their amides: Asn, Asp, Glu, Gln;
3. positively charged residues: His, Arg, Lys;
4. large aliphatic, nonpolar residues: Met, Leu, Ile, Val, (Cys);
5. large aromatic residues: Phe, Trp.

Conservative substitutions are defined as exchanges of an amino acid by another amino acid listed within the same group of the five standard amino acid groups shown above. Three residues are parenthesized because of their special role in protein architecture. Gly is the only residue without a side-chain and therefore imparts flexibility to the chain. Pro has an unusual geometry which tightly constrains the chain Cys can participate in disulfide bonds.

A particularly preferred embodiment of the invention relates to a mutant mouse and human Spinl1 protein having an amino acid sequence as set forth in SEQ ID NO:4 and SEQ ID NO:8, or an isolated fragment of such a protein comprising at least 6, at least 7, at least 8, at least 9, at least 10, at least 15, at least 20, at least 25, at least 30, at least 35, at least 40, at least 50, at least 60, at least 70, at least 80, at least 90, at least 100, at least 150, at least 200, at least 250, at least 300, at least 350, at least 400, at least 450, at least 460, at least 470, at least 480, at least 490, at least 500, at least 510, at least 520, at least 521, at least 522, at least 523, a least 524, at least 525, at least 526 or at least 527 contiguous amino acids of said amino acid sequence, said amino acid sequence comprising an amino acid corresponding to His108.

The invention also provides Spinl1 based chimeric or fusion proteins. As used herein, a “chimeric protein” or “fusion protein” comprises a Spinl1 protein, either wild type or mutant in accordance with the present invention, or a fragment of such protein as defined above, linked to a non-Spinl1 polypeptide (i.e., a polypeptide that does not comprise a Spinl1 protein or a fragment thereof).

In one embodiment, the fusion protein is a OST-Spinl1 fusion protein in which the Spinl1 sequences are fused to the C-terminus of the GST (glutathione-S-transferase) sequences. Such fusion proteins can facilitate the purification of recombinant Spinl1 polypeptides.

In yet another embodiment, the fusion protein is a Spinl1-immunoglobulin fusion protein in which the Spinl1 sequences (i.e., of the mutant or wild type protein or a fragment thereof) are fused to sequences derived from a member of the immunoglobulin protein family, especially Fc region polypeptides. Also contemplated are fusions of Spinl1 sequences (i.e., of the mutant or wild type protein or a fragment thereof) fused to amino acid sequences that are commonly used to facilitate purification or labeling, e.g., polyhistidine tails (such as hexahistidine segments), FLAG tags, HSV-tags, a beta-galactosidase tags and streptavidin.

The amino acid sequences of the present invention may be made by using peptide synthesis techniques well known in the art, such as solid phase peptide synthesis (see, for example, Fields et al., “Principles and Practice of Solid Phase Synthesis” in SYNTHETIC PEPTIDES, A USERS GUIDE, Grant, G. A., Ed., W. H. Freeman Co. NY. 1992, Chap. 3 pp. 77-183; Barlos, K. and Gatos, D. “Convergent Peptide Synthesis” in FMOC SOLID PHASE PEPTIDE SYNTHESIS, Chan, W. C. and White, P. D. Eds., Oxford University Press, New York, 2000, Chap. 9: pp. 215-228) or by recombinant DNA manipulations and recombinant expression, e.g., in a host cell. Techniques for making substitution mutations at predetermined sites in DNA having a known sequence are well known and include, for example, M13 mutagenesis. Manipulation of DNA sequences to produce variant proteins which manifests as substitutional, insertional or deletional variants are conveniently described, for example, in Sambrook et al. (1989), supra.

Animal Model and its Uses

The present invention furthermore provides, for example, a non-human vertebrate animal expressing a Spinl1 protein which is modified compared to the amino acid sequence of the wild type protein at amino acid position 108.

The animal may be a mammalian animal, preferably a rodent, in particular from a genus such as Mus (e.g. mice), Rattus (e.g. rats), Oryctologus (e.g. rabbits) and Mesocricetus (e.g. hamsters) or Bovine (e.g. cow). In a particularly preferred embodiment the animal is a mouse. However, dogs, cats, sheep, and horses are likewise suitable in connection with the invention. The same applies to vertebrates such as fish, in particular Danio rerio or Takifugus rubiens.

The term “phenotype” as used herein refers to one or more morphological, physiological, behavioral and/or biochemical traits possessed by a cell or organism that result from the interaction of the genotype and the environment Thus, the non-human vertebrate animal of the present invention displays one or more readily observable abnormalities compared to the wild type animal. In a preferred embodiment the animal of the invention shows at least 1, at least 2, at least 3, or at least 4 abnormal phenotypical features selected from any of the above categories. In another preferred embodiment, the animal shows a loss of function phenotype. In yet another preferred embodiment, the animal shows a gain of function phenotype.

More generally, the non-human vertebrate animal according to the present invention comprises in the genome of at least some or all of its cells an allele of a gene encoding a protein having at least 63%, at least 65%, at least 70%, at least 75%, at least 80%, at least 85%, at least 90%, at least 95%, at least 98%, or at least 99% amino acid identity compared to the mouse Spinl1 or the human Spinl1 protein according to SEQ ID NO:3 and SEQ ID NO:7, respectively.

The protein mentioned above may be, for example, the corresponding orthologue of the mouse Spinl1 or the human Spinl1 protein according to SEQ ID NO:3 and SEQ ID NO:7 with respect to the animal. It may also be a variant of the mouse Spinl1 or the human Spinl1 protein according to SEQ ID NO:3 and SEQ ID NO:7, or of said orthologue, allelic variant or otherwise, wherein certain amino acids or partial amino acid sequences have been replaced, added, or deleted. In one embodiment, this leads to a variant with a decrease or an abolishment of Spinl1 activity. Alternatively, this leads to a variant with an increased or a constitutive activity compared to the mouse Spinl1 or the human Spinl1 protein according to SEQ ID NO:3 and SEQ ID NO:7, or said orthologue, allelic variant or otherwise.

In a preferred embodiment, the genome of the cells of the animal comprising said allele does not additionally comprise more than one functional allele representing a wild type Spinl1 gene, for example the endogenous wild type Spinl1 gene, or a corresponding wild type orthologue with respect to the animal. Preferably, the genome of the above cells does not additionally comprise any functional allele representing a wild type Spinl1 gene (i.e., no functional allele of endogenous Spinl1 gene or of a corresponding wild type orthologue).

In another preferred embodiment, the genome of the cells of the animal comprising said allele does additionally comprise more than one functional allele respresenting a wild type Spinl1 gene, e.g. the endogenous wild type Spinl1 gene, or a corresponding wild type orthologue with respect to said animal.

The above-mentioned mutated allele comprised in the genome of the cells of the non-human vertebrate animal comprises a mutation which, if present in the genome of all or essentially all cells of said animal in a homozygous manner, in particular in the animal's adipocytes, results in a phenotype associated with an alteration in fat metabolism compared to the corresponding wild type animal. It will be appreciated that this mutation may reside in either the coding or the non-coding region of the allele.

In view of the fact that the present invention for the first time draws a connection between Spinl1 and agents capable of modulating Spinl1 activity with fat metabolism, it will be apparent to the skilled artisan that other genes and their products which in turn modulate Spinl1 gene expression or the activity of the Spinl1 protein may likewise be expected to affect the phenotypes and physiological and medical conditions associated with an alteration in fat metabolism. Accordingly, the present invention provides in a further aspect a non-human vertebrate animal comprising in the genome of at least some or all of its cells an allele of a gene coding for a protein which affects expression or activity of the Spinl1 protein of the animal, said allele comprising a mutation which, if present in the genome of all or essentially all cells of said animal in a homozygous manner, results in a phenotype associated with an alteration in fat metabolism compared to the corresponding wild type animal.

The term “modulation” in the present invention may comprise any alteration in Spinl1 activity, e.g., a reduction, or complete inhibition, or an increase, e.g., a constitutive or temporal activation.

The gene referred to above in connection with the animal according to the invention is preferably an endogenous gene with respect to said animal. The gene may, however, also be a heterologous gene with respect to said animal. In preferred embodiments, the gene will encode a protein which is an orthologue of the Spinl1 proteins defined by SEQ ID NO:3 and SEQ ID NO:7 with respect to said animal. For example, a mouse according to the present invention may be one wherein the endogenous mouse Spinl1 gene has been replaced by a mutated human Spinl1 gene, e.g., by a mutated allele encoding an Spinl1 protein as described above, particularly a mutant Spinl1 protein having SEQ ID NO: 4 or SEQ ID NO: 8. Likewise, a rat according to the present invention may be one wherein the endogenous rat Spinl1 gene has been replaced by a mutated mouse Spinl1 gene, e.g., by a mutated allele encoding an Spinl1 protein as described in this invention.

As will be apparent from the previous explanations, the non-human vertebrate animal according to the invention may also be a transgenic animal, i.e., the mutated allele of the gene may represent DNA that is heterologous with respect to the genomic DNA of said animal, or it may be mutated by virtue of the insertion of DNA that is heterologous with respect to the genomic DNA of said animal. Heterologous DNA may be inserted, for example, by the method of targeting vector-mediated homologous recombination at the Spinl1 genomic DNA locus in mouse embryonic stem cells, resulting in a replacement of the endogenous Spinl1 allele by heterologous DNA, as will be appreciated by those skilled in the art. Transgenic animals may then be generated by subsequent reimplanting embryonic stem cells carrying the heterologous DNA into a mouse blastocyst and subsequent breeding.

The endogenous promoter of the Spinl1 gene or the gene affecting its expression or function may be replaced by a heterologous promoter, e.g., a promoter imposing a different tissue specificity of expression upon the gene, or a promoter that is inducible by chemical or physical means.

The non-human vertebrate animal according to the invention may also be a “knock out” animal with respect to the Spinl1 gene or the gene affecting expression or function of the Spinl1 protein. In these animals, the above-mentioned mutation results in the reduction or complete abolishment of expression of said gene.

The mutated allele may be present in germ cells or somatic cells of the non-human vertebrate animal, or both. In a preferred embodiment, the genome of said cells is homozygous with respect to said allele.

The present invention further provides for inbred successive lines of animals carrying the mutant Spinl1 nucleic acid of the present invention that offer the advantage of providing a virtually homogeneous genetic background. A genetically homogeneous line of animals provides a functionally reproducible model system for conditions or symptoms associated with alterations in fat metabolism.

In a particularly preferred embodiment the non-human vertebrate animal according to the invention expresses in at least some of its cells, preferably the adipocytes, a mutated allele encoding a Spinl1 protein as described in connection with this invention.

The animals of the invention can be produced by using any technique known to the person skilled in the art; including but not limited to micro-injection, electroporation, cell gun, cell fusion, nucleus transfer into anucleated cells, micro-injection into embryos of teratocarcinoma stem cells or functionally equivalent embryonic stem cells. The animals of the present invention may be produced by the application of procedures, which result in an animal with a genome that incorporates/integrates exogenous genetic material in such a manner as to modify or disrupt the function of the normal Spinl1 gene or protein. A preferred procedure for generating an animal of this invention is one according to Example 1.

Alternatively, the procedure may involve obtaining genetic material, or a portion thereof, which encodes a wild type Spinl1 protein, as described in Example 13. The isolated native sequence is then genetically manipulated by the insertion of any of the mutations described and claimed in accordance with the present invention, e.g., a mutation appropriate to replace, e.g., the residue at position 108 of the amino acid sequence shown in SEQ ID NO:3 or SEQ ID NO:7, or appropriate to insert additional amino acids normally not present in the amino acid sequences of SEQ ID NO: 3 or SEQ ID NO: 7 of the mouse or human Spinl1 proteins (see Example 9).

The manipulated construct may then be inserted into embryonic stem cells, e.g., by electroporation. The cells subjected to the procedure are screened to find positive cells, i.e., cells, which have integrated into their genome the desired construct encoding an altered Spinl 1. The positive cells may be isolated, cloned (or expanded) and injected into blastocysts obtained from a host animal of the same species or a different species. For example, positive cells are injected into blastocysts from mice, the blastocysts are then transferred into a female host animal and allowed to grow to term, following which the offspring of the female are tested to determine which animals are transgenic, i.e., which animals have an inserted exogenous mutated DNA sequence. One suitable method involves the introduction of the recombinant gene at the fertilized oocyte stage ensuring that the gene sequence will be present in all of the germ cells and somatic cells of the “founder” animal. The term “founder animal” as used herein means the animal into which the recombinant gene was introduced at the one cell embryo stage.

The animals of the invention can also be used as a source of primary cells from a variety of tissues, for cell culture experiment, including, but not limited to, the production of immortalized cell lines by any methods known in the art, such as retroviral transformation. Such primary cells or immortalized cell lines derived from any one of the non-human vertebrate animals described and claimed herein are likewise within the scope of the present invention. Such immortalized cells from these animals may advantageously exhibit desirable properties of both normal and transformed cultured cells, i.e., they will be normal or nearly normal morphologically and physiologically, but can be cultured for long, and perhaps indefinite periods of time. The primary cells or cell lines derived thereof may furthermore be used for the construction of an animal model according to the present invention.

In other embodiments cell lines according to the present invention may be prepared by the insertion of a nucleic acid construct comprising the nucleic acid sequence of the invention or a fragment thereof comprising the codon imparting the above-described phenotype to the animal model of the invention. Suitable cells for the insertion include primary cells harvested from an animal as well as cells, which are members of an immortalized cell line. Recombinant nucleic acid constructs of the invention, described below, may be introduced into the cells by any method known in the art, including but not limited to, transfection, retroviral infection, micro-injection, electroporation, transduction or DEAE-dextran. Cells, which express the recombinant construct, may be identified by, for example, using a second recombinant nucleic acid construct comprising a reporter gene, which is used to produce selective expression. Cells that express the nucleic acid sequence of the invention or a fragment thereof may be identified indirectly by the detection of reporter gene expression.

It will be appreciated that the non-human vertebrate animals of the invention are useful in various respects in connection with adipocyte function or dysfunction and fat metabolism related phenotypes and medical conditions associated with an alteration in fat metabolism. The term “medical condition associated with an alteration in fat metabolism” as used throughout the present application preferably refers to a medical condition selected from the group consisting of obesity; obesity and diabetes, particularly type II diabetes; and diabetes, particularly type II diabetes.

Accordingly, one aspect of the present invention is the use of the non-human vertebrate animal for the identification of a protein or nucleic acid diagnostic marker for an alteration in fat metabolism. Also within the scope of the present invention is the use of the animal as a model for studying the molecular mechanisms of, or physiological processes associated with an alteration in fat metabolism, or for the identification and testing of an agent useful in the prevention, amelioration, or treatment of a medical condition associated with an alteration in fat metabolism.

Further uses of the non-human vertebrate animals described herein which form additional aspects of the present invention are those relating to studying the molecular mechanisms of, or physiological processes associated with, conditions associated with, or affected by, reduced activity or undesirable activity of endogenous Spinl1. Likewise, conditions associated with reduced expression, reduced production or undesirable production of endogenous Spinl1 may be analyzed. The non-human vertebrate animals are also useful for the identification and testing of an agent useful in the prevention, amelioration, or treatment of these conditions.

It will also be appreciated that the non-human vertebrate animals described herein will be highly useful as a model system for the screening and identification of binding partners, particularly ligands of the Spinl1 protein or genes or proteins regulated by Spinl1 activity and/or deregulated by altered Spinl1 expression. Such agents may be, for example, small molecule drugs, peptides or polypeptide, or nucleic acids. The term “small molecule drug” as used throughout the present application refers to drug molecules preferably having a molecular weight of no more than 2,000 Dalton, more preferably no more than 1500 Dalton, even more preferably no more than 1000 Dalton, and most preferably no more than 500, 400, 300 or even 200 Dalton.

In other embodiments, the non-human vertebrate animal described herein may be used for studying the molecular mechanisms of, or physiological processes associated with, conditions associated with, or affected by an altered plasma insulin level or an altered insulin sensitivity.

Further uses of the non-human vertebrate animals described herein which form another additional aspects of the present invention are those relating to studying the molecular mechanisms of, or physiological processes associated with, conditions associated with, or affected by altered leptin level or altered leptin sensitivity.

It will furthermore be apparent from the above that the non-human vertebrate animals described herein will be highly useful for identifying protein or nucleic acid diagnostic markers, such as diagnostic markers relating to genes or gene products that play a role in the early phase, the intermediate phase, and/or the late phase of medical conditions associated with an alteration in fat metabolism, e.g., obesity; obesity and diabetes, particularly type II diabetes; or diabetes, particularly type II diabetes, or diagnostic markers for diseases associated with Spinl1 deficiency or over-expression. It will be appreciated that such diagnostic markers may relate to the Spinl1 gene or its protein product. However, it will be understood that the non-human vertebrate animal according to the present invention can also be used to identify markers relating to other genes or gene products that affect Spinl1 gene or protein expression or function, or the expression or function of which is affected by the Spinl1 protein. Moreover, since the non-human vertebrate animal of the invention represents a highly useful model system for studying the pathogenesis of medical conditions associated with an alteration in fat metabolism, it will be appreciated that it may also be used to identify disease-relevant markers relating to genes or gene products that do not directly affect Spinl1 gene or protein expression or activity, or the expression or activity of which is not directly affected by the Spinl1 protein. It will be appreciated that the above-mentioned uses represent further aspects of the present invention.

Finally, it will be appreciated from the above that the non-human vertebrate animals described herein will be highly useful for identifying binding partners, particularly ligands of the Spinl1 protein, or upstream or downstream genes or proteins regulated by the Spinl1 protein or gene activity, and deregulated in disorders associated with Spinl1 deficiency or over-expression.

Antisense Nucleic Acids

A preferred nucleic acid according to the present invention is an antisense nucleic acid comprising a nucleotide sequence which is complementary to a part of an mRNA encoding a mutant protein according to the present invention, said part encoding an amino acid sequence comprising the amino acid or amino acid sequence which corresponds to the mutation described in more detail in connection with said mutant protein.

A further preferred antisense nucleic acid is one comprising a nucleotide sequence which is complementary to a part of an mRNA encoding the mouse Spinl1 or the human Spinl1 protein according to SEQ ID NO:3 and SEQ ID NO:7, respectively, or an orthologue thereof having at least 63%, 65%, 70%, 75%, 80%, 85%, 90%, 95%, 98%, or 99% amino acid identity compared to the mouse Spinl1 or the human Spinl1 protein as defined above, said part being a non-coding part and comprising a sequence corresponding to a mutation in the gene coding for said protein or orthologue which affects expression of said protein or orthologue.

Yet a further preferred antisense nucleic acid is one comprising a nucleotide sequence which is complementary to a part of an mRNA encoding a protein which affects expression or activity of the mouse Spinl1 or the human Spinl1 protein as defined in the two preceding paragraphs.

A further preferred antisense nucleic acid is one comprising a nucleotide sequence which is complementary to a part of an mRNA encoding the mouse Spinl1 or the human Spinl1 protein according to SEQ ID NO:3 and SEQ ID NO:7, respectively, or an orthologue thereof having at least 63%, 65%, 70%, 75%, 80%, 85%, 90%, 95%, 98%, or 99% amino acid identity compared to the mouse Spinl1 or the human Spinl1 protein.

Yet a further preferred antisense nucleic acid is one comprising a nucleotide sequence which is complementary to a part of an mRNA encoding a protein which affects expression or activity of the mouse Spinl1 or the human Spinl1 protein according to SEQ ID NO:3 and SEQ ID NO:7, respectively, or an orthologue thereof having at least 63%, 65%, 70%, 75%, 80%, 85%, 90%, 95%, 98%, or 99% amino acid identity compared to the mouse Spinl1 or the human Spinl1 protein according to SEQ ID NO:3 and SEQ ID NO:7, respectively.

A further preferred antisense nucleic acid is one which is a DNA, an RNA, or a synthetic nucleic acid analog, such as a peptide nucleic acid.

In a preferred embodiment, the antisense nucleic acid is capable of hybridizing to the “mRNA via the complementary nucleotide sequence under physiological conditions, in particular the preferred physiological conditions defined above. In this case, the antisense RNA is inter alia suitable to be used in connection with the methods and uses of the present invention that relate to the prevention, treatment, or amelioration of a medical condition associated with an alteration in fat metabolism. In another preferred embodiment, the antisense RNA according to the present invention is capable of hybridizing to said mRNA under high stringency conditions, in particular the preferred high stringency conditions defined above.

The antisense nucleic acid may be a ribozyme comprising a catalytic region; suitably, the catalytic region enables the antisense RNA to specifically cleave the mRNA to which the antisense RNA hybridizes.

In case of an antisense nucleic acid which is complementary to a part of an mRNA encoding a mutant Spinl1 protein, it may be advantageous that the antisense nucleic acid of the invention hybridizes more effectively to its target mRNA than to an mRNA encoding the same protein which, however, corresponds to the wild type mouse Spinl1 or human Spinl1 protein according to SEQ ID NO:3 and SEQ ID NO:7 in respect of the mutated amino acid sequence. Also preferred are antisense nucleic acids which hybridize more effectively to their target mRNA than to the mRNA encoded by the wild type genes encoding the mouse Spinl1 protein or the human Spinl1 protein according to SEQ ID NO:3 and SEQ ID NO:7, respectively, or the wild type gene encoding the corresponding orthologue. Preferred are in addition antisense nucleic acids which hybridize more effectively to their target mRNA than to the mRNA encoded by the wild type gene of the corresponding protein which affects expression or activity of the mouse Spinl1 or the human Spinl1 protein according to SEQ ID NO:3 and SEQ ID NO:7, respectively.

Prokaryotic and eukaryotic host cells transformed with the above antisense nucleic acids are likewise within the scope of the present invention.

An inventive therapeutic method of the invention comprises administering to a mammalian subject, preferably to a human subject, a Spinl1 DNA construct comprising a sequence encoding an antisense nucleic acid as defined above to inhibit enogenous wild type Spinl1 activity, or to compensate for increased or aberrant expression or activity of an endogenous mutant Spinl1 protein as described herein. The construct is administered to cells, e.g., adipocytes, or tissues using known nucleic acid transfection techniques, as described herein. A Spinl1 antisense nucleic acid specific for a Spinl1 wild type or mutant gene will decrease or knockdown Spinl1 transcription products, which will lead to reduced polypeptide production of said Spinl1, resulting in reduced Spinl1 polypeptide activity in the cells or tissues of said subject.

Interfering RNA

In one aspect of the invention, Spinl1 wild type or mutant gene expression can be attenuated or abolished by RNA interference. One approach well-known in the art is short interfering RNA (siRNA) mediated gene silencing. In this case, expression products of the Spinl1 gene are targeted by specific double stranded Spinl1 derived siRNA nucleotide sequences that are complementary to at least a 19-25 nt long segment of the Spinl1 gene transcript, including the 5′ untranslated (UT) region, the open reading frame (ORF), or the 3′ UT region. See, for example, PCT applications WO 00/44895, WO 99/32619, WO 01/75164, WO 01/92513, WO 01/29058, WO 01/89304, WO 02/16620, and WO 02/29858, each incorporated by reference herein in their entirety. Targeted genes can be a Spinl1 gene, or an upstream or downstream modulator of Spinl1 gene expression or protein activity. For example, expression of a phosphatase or kinase specific for Spinl1 may be targeted by an siRNA.

According to the methods of the present invention, Spinl1 gene expression is silenced using short interfering RNA. A Spinl1 polynucleotide according to the invention includes an siRNA polynucleotide. Such a Spinl1 siRNA can be obtained using a Spinl1 polynucleotide sequence, for example, by processing the Spinl1 ribopolynucleotide sequence in a cell-free system, such as but not limited to a Drosophila extract, or by transcription of recombinant double stranded Spinl1 RNA or by chemical synthesis of nucleotide sequences homologous to a Spinl1 sequence. See, e.g., Tuschl, Zamore, Lehmann, Bartel and Sharp (1999), Genes & Dev. 13: 3191-3197, incorporated herein by reference in its entirety (Tuschl, Zamore, Lehmann, Bartel, and Sharp 1999a). When synthesized, a typical 0.2 micromolar-scale RNA synthesis provides about 1 milligram of siRNA, which is sufficient for 1000 transfection experiments using a 24-well tissue culture plate format The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides. Using 2′-deoxynucleotides in the 3′ overhangs is as efficient as using ribonucleotides, but deoxyribonucleotides are often cheaper to synthesize and are most likely more nuclease resistant A recombinant expression vector of the invention comprises a Spinl1 DNA molecule cloned into an expression vector comprising operatively-linked regulatory sequences flanking the Spinl1 sequence in a manner that allows for expression (by transcription of the DNA molecule) of both strands. An RNA molecule that is antisense to Spinl1 mRNA is transcribed by a first promoter (e.g., a promoter sequence 3′ of the cloned DNA) and an RNA molecule that is the sense strand for the Spinl1 mRNA is transcribed by a second promoter (e.g., a promoter sequence 5′ of the cloned DNA). The sense and antisense strands may hybridize in vivo to generate siRNA constructs for silencing of the Spinl1 gene. Alternatively, two constructs can be utilized to create the sense and anti-sense strands of an siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is an siRNA. The regulatory sequences flanking the Spinl1 sequence may be identical or may be different, such that their expression may be modulated independently, or in a temporal or spatial manner.

In a specific embodiment, siRNAs are transcribed intracellularly by cloning the Spinl1 gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type m class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stem-loop transcript.

siRNA vectors appear to have an advantage over synthetic siRNAs where long term knock-down of expression is desired. Cells transfected with an siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In another specific ambodiment, siRNAs are transcribed intracellularly after cloning templates into the vector system pSilencer™ 2.1-U6 neo (Ambion Inc., Austin, Tex., USA), followed by subsequent transfection of Spinl1 expressing cells. Double stranded oligonucleotides, e.g., oligonucleotides of 63-67 base pairs in length, representing the templates cloned into the vector system pSilencer™ 2.1-U6 neo and targeting, e.g., the particular nucleotide sequences described in SEQ ID NOS:43, 44, 45, 46, 47, or 48, may be synthesized and processed. The 63- to 67mer oligonucleotides (see, e.g., SEQ ID NOS:49, 50, 51, 52, 53, 54, 55, 56, 57, 58, 59, and 60) advantageously comprise a first Spinl1 nucleotide to be transcribed (guanidine), a loop sequence of 9 bases, a sequence which is revers complementary to a target sequence, six thymidine residues (serving as transcription stop signal for the RNA Polymerase III), a sequence motif GGAA recommended by AMBION Inc., and sequences for generating the required restriction enzyme cloning sites BamHI and HindIII. Reverse complementary oligonucleotides are annealed, e.g., as follows: the oligonucleotide of SEQ ID NO:49 to the oligonucleotide of SEQ ID NO:50, the oligonucleotide of SEQ ID NO:51 to the oligonucleotide of SEQ ID NO:52, the oligonucleotide of SEQ ID NO:53 to the oligonucleotide of SEQ ID NO:54, the oligonucleotide of SEQ ID NO:55 to the oligonucleotide of SEQ ID NO:56, the oligonucleotide of SEQ ID NO:57 to the oligonucleotide of SEQ ID NO:58, and the oligonucleotide of SEQ ID NO:59 to the oligonucleotide of SEQ ID NO:60. Double stranded oligonucleotides are subsequently cloned into pSilencer™ 2.1-U6 neo (AMBION Cat# 5764), following the manufacturer's instruction manual for Cat. #5764, 5770.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A Spinl1 mRNA region to be targeted by siRNA is generally selected from a desired Spinl1 wild type or mutant sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See (Elbashir, Martinez, Patkaniowska, Lendeckel, and Tuschl 2001b). Hence, consideration should be taken to accommodate SNPs, polymorphisms, allelic variants or species-specific variations when targeting a desired gene.

A complete Spinl siRNA experiment should include the proper negative control. Negative control siRNA should have the same nucleotide composition as the Spinl1 siRNA but lack significant sequence homology to the genome. Typically, one would scramble the nucleotide sequence of the Spinl1 siRNA and do a homology search to make sure it lacks homology to any other gene.

Two independent Spinl1 siRNA duplexes can be used to knock-down a target Spinl1 gene. This helps to control for specificity of the silencing effect In addition, expression of two independent genes can be simultaneously knocked down by using equal concentrations of different Spinl1 siRNA duplexes. Availability of siRNA-associating proteins is believed to be more limiting than target mRNA accessibility.

A targeted Spinl1 region is typically a sequence of two adenines (AA) and two thymidines (TT) divided by a spacer region of nineteen (N19) residues (e.g., AA(N19)TT). A desirable spacer region has a G/C-content of approximately 30% to 70%, and more preferably of about 50%. If the sequence AA(N19)TT is not present in the target sequence, an alternative target region would be AA(N21). The sequence of the Spinl1 sense siRNA corresponds to (N19)TT or N21, respectively. In the latter case, conversion of the 3′ end of the sense siRNA to TT can be performed if such a sequence does not naturally occur in the Spinl1 polynucleotide. The rationale for this sequence conversion is to generate a symmetric duplex with respect to the sequence composition of the sense and antisense 3′ overhangs. Symmetric 3′ overhangs may help to ensure that the siRNPs are formed with approximately equal ratios of sense and antisense target RNA-cleaving siRNPs (see (Elbashir, Lendeckel, and Tuschl 2001a) incorporated by reference herein in its entirety). The modification of the overhang of the sense sequence of the siRNA duplex is not expected to affect targeted mRNA recognition, as the antisense siRNA strand guides target recognition.

Alternatively, if the Spinl1 target mRNA does not contain a suitable AA(N21) sequence, one may search for the sequence NA(N21). Further, the sequence of the sense strand and antisense strand may still be synthesized as 5′ (N19)TT, as it is believed that the sequence of the 3′-most nucleotide of the antisense siRNA does not contribute to specificity. Unlike antisense or ribozyme technology, the secondary structure of the target mRNA does not appear to have a strong effect on silencing. See (Harborth, Elbashir, Becher, Tuschl, and Weber 2001), incorporated herein by reference in its entirety.

Transfection of Spinl1 siRNA duplexes can be achieved using standard nucleic acid transfection methods, for example, OLIGOFECTAMINE Reagent (commercially available from Invitrogen). An assay for Spinl1 gene silencing is generally performed approximately 2 days after transfection. No Spinl1 gene silencing has been observed in the absence of transfection reagent, allowing for a comparative analysis of the wild type and silenced Spinl1 phenotypes. In a specific embodiment, for one well of a 24-well plate, approximately 0.84 μg of the siRNA duplex is generally sufficient. Cells are typically seeded the previous day, and are transfected at about 50% confluence. The choice of cell culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g. inversion versus vortexing) are also critical. Low transfection efficiencies are the most frequent cause of unsuccessful Spinl1 silencing. The efficiency of transfection needs to be carefully examined for each new cell line to be used. Preferred cells are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense Spinl1 siRNA will have no effect on Spinl1 silencing, and 0.84 μg antisense siRNA has a weak silencing effect when compared to 0.84 μg of duplex siRNAs. Control experiments again allow for a comparative analysis of the wild type and silenced Spinl1 phenotypes. To control for transfection efficiency, targeting of common proteins is typically performed, for example targeting of lamin A/C or transfection of a CMV-driven EGFP-expression plasmid (e.g. commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted Spinl1 polynucleotide in a cell, a knock-down phenotype may become apparent after 1 to 3 days, or even later. In cases where no Spinl1 knock-down phenotype is observed, depletion of the Spinl1 polynucleotide may be observed by immunofluorescence or Western blotting. If the Spinl1 polynucleotide is still abundant after 3 days, cells need to be split and transferred to a fresh 24-well plate for re-transfection. If no knock-down of the targeted protein (Spinl1 or a Spinl1 upstream or downstream gene) is observed, it may be desirable to analyze whether the target mRNA was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable Spinl1 protein may exist in the cell. Multiple transfection in sufficiently long intervals may be necessary until the target protein is finally depleted to a point where a phenotype may become apparent If multiple transfection steps are required, cells are split 2 to 3 days after transfection. The cells may be transfected immediately after splitting.

An inventive therapeutic method of the invention contemplates administering a Spinl1 siRNA construct as therapy to inhibit wild type Spinl1 activity or to compensate for increased or aberrant expression or activity of mutant Spinl1 as described herein. The Spinl1 ribopolynucleotide is obtained and processed into siRNA fragments as described. The Spinl1 siRNA is administered to cells or tissues using known nucleic acid transfection techniques, as described above. An Spinl1 siRNA specific for an Spinl1 gene will decrease or knockdown Spinl1 transcription products, which will lead to reduced Spinl1 polypeptide production, resulting in reduced Spinl1 polypeptide activity in the cells or tissues.

Particularly preferred in connection with the present invention are siRNAs comprising a double stranded nucleotide sequence wherein one strand is complementary to an at least 19, 20, 21, 22, 23, 24, or 25 nucleotide long segment of an mRNA encoding a mutant protein of the invention as described herein, said segment encoding an amino acid sequence comprising the amino acid or amino acid sequence which corresponds to any of the mutations defined previously or hereinafter in connection with the mutant protein.

Also preferred are siRNAs wherein said strand is complementary to an at least 19, 20, 21, 22, 23, 24, or 25 nucleotide long segment of an mRNA encoding the mouse Spinl1 or the human Spinl1 protein according to SEQ ID NO:3 and SEQ ID NO:7, respectively, or an orthologue thereof having at least 63%, 65%, 70%, 75%, 80%, 85%, 90%, 95%, 98%, or 99% amino acid identity compared to the mouse Spinl1 or the human Spinl1 protein as defined above, said segment being a non-coding segment and comprising a sequence corresponding to a mutation in the gene coding for said protein or orthologue which affects expression of said protein or orthologue.

Yet a further preferred antisense nucleic acid is one comprising a nucleotide sequence which is complementary to a part of an mRNA encoding a protein which affects expression or activity of the mouse Spinl1 or the human Spinl1 protein as defined in the two preceding paragraphs.

Particularly preferred in connection with the present invention are siRNAs, wherein said strand is complementary to an at least 19, 20, 21, 22, 23, 24, or 25 nucleotide long segment of an mRNA encoding the mouse Spinl1 or the human Spinl1 protein according to SEQ ID NO:3 and SEQ ID NO:7, respectively, or an orthologue thereof having at least 63%, 65%, 70%, 75%, 80%, 85%, 90%, 95%, 98%, or 99% amino acid identity compared to the mouse Spinl1 or the human Spinl1 protein as defined above.

Furthermore preferred are siRNAs wherein said strand is complementary to an at least 19, 20, 21, 22, 23, 24, or 25 nucleotide long segment of an mRNA encoding a protein which affects expression or function of the mouse Spinl1 or the human Spinl1 protein according to SEQ ID NO:3 and SEQ ID NO:7, respectively, or an orthologue thereof having at least 63%, 65%, 70%, 75%, 80%, 85%, 90%, 95%, 98%, or 99% amino acid identity compared to the mouse Spinl1 or the human Spinl1 protein according to SEQ ID NO:3 and SEQ ID NO:7, respectively.

Particularly preferred are siRNAs wherein said strand is complementary to a 21 nucleotide segment of an mRNA encoding mouse Spinl1 protein, as described in SEQ ID NOS:43, 44, 45, 46, or 48. Also particularly preferred is an siRNA wherein said strand is complementary to a 20 nucleotide segment of an mRNA encoding mouse Spinl1 protein, as described in SEQ ID NO:47.

The above-mentioned segment may include sequences from the 5′ untranslated (UT) region. Alternatively, or in addition, it may include sequences corresponding to the open reading frame (ORF). Again alternatively or in addition, it may include sequences from the 3′ untranslated (UT) region.

Prokaryotic and eukaryotic host cells transformed with the above siRNAs are likewise within the scope of the present invention.

The present invention also encompasses a method of treating a disease or condition associated with the presence of a Spinl1 protein in an individual comprising administering to the individual an siRNA construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the Spinl1 gene function is not correlated with a known phenotype, a control sample of cells or tissues from healthy individuals provides a reference standard for determining Spinl1 expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The Spinl1 ribopolynucleotide is used to produce siRNA constructs, that are specific for the Spinl1 gene product. These cells or tissues are treated by administering Spinl1 siRNAs to the cells or tissues by methods described for the transfection of nucleic acids into a cell or tissue, and a change in Spinl1 polypeptide or polynucleotide expression is observed in the subject sample relative to the control sample, using the assays described. This Spinl1 gene knockdown approach provides a rapid method for determination of a Spinl1-phenotype in the treated subject sample. The Spinl1-phenotype observed in the treated subject sample thus serves as a marker for monitoring the course of a disease state during treatment

Aptamers

The invention furthermore provides aptamers specifically binding the proteins described herein. As known from literature, aptamers bind their ligand with high specificity and affinities in the low nanomolar range, with K(D) values ranging between 12 nM and 130 nM. Preferably, the specificity of the aptamers is sufficient so that they do not bind any other protein in the cell. Preferred aptamers bind to the Spinl1 muteins of the present invention or a portion thereof comprising a mutation as described herein, i.e., a substitution of amino acid 108. Another preferred aptamer binds to the wild type Spinl1 protein or a portion thereof, according to SEQ ID NO: 3 and SEQ ID NO:7. Aptamers are macromolecules composed of nucleic acid, such as RNA or DNA, that tightly bind proteins, e.g., Spinl1 wild type proteins or Spinl1 muteins.

Antibodies

A further aspect of the present invention is an antibody specifically recognizing an epitope in a human Spinl1 protein of a human subject known not to have a medical condition associated with an alteration in fat metabolism, preferably the protein according to SEQ ID NO: 7 or an allelic variant thereof.

Another aspect of the present invention is an antibody specifically recognizing an epitope in a mutant Spinl1 protein as described herein, wherein said epitope comprises the amino acid or the amino acid sequence in said protein which corresponds to the mutation described in connection with these mutant proteins.

Also included in the invention are antibodies specifically recognizing fragments of the mutant Spinl1 polypeptides (including amino terminal fragments), as well as antibodies to the fusion proteins containing Spinl1 mutant polypeptides or fragments of Spinl1 mutant polypeptides according to the invention.

Any protein of the invention, or a derivative, fragment, analog, homolog or orthologue thereof, may be utilized as an immunogen in the generation of antibodies that immunospecifically bind these protein components.

The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, e.g., polyclonal, monoclonal, chimeric, single chain, F_{ab}, F_{ab′}and F_{(ab′)2} fragments, and a F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

A Spinl1 wild type or mutant polypeptide of the invention or a portion or fragment thereof may be intended to serve as an antigen, and additionally can be used as an immunogen to generate antibodies that immunospecifically bind the antigen, using standard techniques for polyclonal and monoclonal antibody preparation. Antigenic peptide fragments of the antigen for use as immunogens includes, e.g., at least 7 amino acid residues of the amino acid sequence of the human Spinl1 wild type protein, e.g of SEQ ID NO: 7, or the mutant Spinl1 protein, e.g. SEQ ID NO:8. Preferably, the antigenic peptide comprises at least 10 amino acid residues, or at least 15 amino acid residues, or at least 20 amino acid residues, or at least 30 amino acid residues of said Spinl1 proteins. Preferred epitopes encompassed by the antigenic peptide are regions of the protein that are located on its surface; commonly these are hydrophilic regions. Antigenic peptide fragments of mutant Spinl1 proteins for use as immunogens includes, e.g., at least 7 amino acid residues of the amino acid sequence of the mutated region such as the region comprising tyrosine 108 of SEQ ID NO: 3 or SEQ ID NO: 7 of the mouse Spinl1 or the human Spinl1 protein.

In certain embodiments of the invention, at least one epitope encompassed by the antigenic peptide is a region of a Spinl1 polypeptide that is located on the surface of the protein, e.g., a hydrophilic region. A hydrophobicity analysis of a wild type or mutant Spinl1 polypeptide will indicate which regions of said Spinl1 protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g. (Hopp and Woods 1981; Kyte and Doolittle 1982). Antibodies that are specific for one or more domains within an antigenic protein, or derivatives, fragments, analogs or homologs thereof, are also provided herein.

Various procedures known within the art may be used for the production of polyclonal or monoclonal antibodies directed against a protein of the invention, or against derivatives, fragments, analogs, homologues or orthologues thereof. See, for example, ANTIBODIES: A LABORATORY MANUAL, Harlow and Lane (1988) Cold Spring Harbor Laboratory Press, Cold Spring Harbor, N.Y. Some of these antibodies are discussed below.

Polyclonal Antibodies

For the production of polyclonal antibodies, various suitable host animals (e.g., rabbit, goat, mouse or another mammal) may be immunized by one or more injections with the protein of the invention, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor.

The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g, from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography.

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it

Monoclonal antibodies can be prepared using hybridoma methods, such as those described by Kohler and Milstein (Kohler and Milstein 1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, MONOCLONAL ANTIBODIES: PRINCIPLES AND PRACTICE, Academic Press, (1986) pp. 59-103. Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies ((Kozbor, Tripputi, Roder, and Croce 1984) Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Rodbard (Munson and Rodbard 1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; (Morrison 1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., 1986; Riechmann et al., 1988b; Verhoeyen et al., 1988a; Riechmann et al., 1988a; Verhoeyen et al., 1988b), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues, which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988b; Riechmann et al., 1988a).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (Cote et al., 1983) or by transforming human B-cells with Epstein Barr Virus in vitro (see Cole, et al. (1985) In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, 1992; Marks et al., 1991a; Marks et al., 1991b). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in here: Fishwild et al., 1996b; Lonberg et al., 1994b; Lonberg and Huszar, 1995b; Marks et al., 1992; Morrison, 1994b; Neuberger, 1996b; Fishwild et al., 1996a; Lonberg et al., 1994a; Lonberg and Huszar, 1995a; Morrison, 1994a; Neuberger, 1996a Human antibodies may additionally be produced using transgenic nonhuman animals which are modified so as to produce fully human antibodies rather than the animal's endogenous antibodies in response to challenge by an antigen. See PCT publication WO 94/02602. The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells, which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (Huse et al., 1989) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) F_{ab} fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art Traditionally, the recombinant production of bispecific antibodies is based on the co-expression of two immunoglobulin heavy-chain/light-chain pairs, where the two heavy chains have different specificities (Milstein and Cuello, 1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829 and in Traunecker et al. (Traunecker et al., 1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al. (Suresh et al., 1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al. (Brennan et al., 1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al. (Shalaby et al., 1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers (Kostelny et al., 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology (Holliger et al., is 1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported (Gruber et al., 1994).

Antibodies with more than two valencies are contemplated. For example, trispecific antibodies can be prepared (Tutt et al., 1991).

Exemplary bispecific antibodies can bind to two different epitopes, at least one of which originates in the protein antigen of the invention. Bispecific antibodies can also be used to direct various agents to cells, which express a particular antigen. These antibodies possess an antigen-binding arm and an arm, which binds an agent such as a radionuclide chelator (e.g., EOTUBE, DPTA, DOTA, or TETA).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving cross-linking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC) (Caron et al., 1992; Shopes, 1992a; Shopes, 1992b). Homodimeric antibodies with enhanced anti-tumor activity can also be prepared using heterobifunctional cross-linkers as described in Wolff et al. (Wolff et al., 1993). Alternatively, an antibody can be engineered that has dual Fc regions and can thereby have enhanced complement lysis and ADCC capabilities (Stevenson et al., 1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described (Vitetta, Krolick, Miyama-Inaba, Cushley, and Uhr 1983). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immonoconjugates according to the present invention are furthermore those comprising an antibody as described above conjugated to an imaging agent. Imaging agents suitable in this regard are, for example, again certain radioactive isotopes. Suitable in this regard are ¹⁸F, ⁶⁴Cu ⁶⁷Ga, ⁶⁸Ga, ^{99m}Tc, ¹¹¹In, ¹²³I, ¹²⁵I, ¹³¹I, ¹⁶⁹Yb, ¹⁸⁶Re, and ²⁰¹Tl. Particularly preferred in this regard is ^{99m}Tc. The radioactive isotopes will suitably be conjugated to the antibody via a chelating group that is covalently attached to the antibody and is capable of chelating the radioactive isotope.

Anticalins

Anticalins are engineered proteins with antibody-like binding functions derived from natural lipocalins as a scaffold. These small monomeric proteins of only 150 to 190 amino acids might offer competitive advantages over antibodies, i.e., in increased binding specificity and improved tissue penetration such as solid tumors. Their set of four loops can be easily manipulated at the genetic level (Weiss and Lowmann, 2000; Skerra, 2001). As known from literature, anticalins bind their ligand with high specificity and affinities in low molecular range, with K(D) values ranging from 12 nM to 35 nM. A preferred anticalin specifically binds to wild type Spinl1 proteins, according to SEQ ID NO:3 or SEQ ID NO:7; or binds to the Spinl1 muteins of the present invention or a portion thereof comprising a mutation as described herein, i.e., a substitution of amino acid 108.

Pharmaceutical Compositions

The invention also includes pharmaceutical compositions containing agents that can modulate Spinl1 activity or expression. In this connection, Spinl1 may be the wild type Spinl1 protein or the mutant Spinl1 protein, particularly mutant proteins with an increased activity. These agents furthermore include biomolecules such as proteins, kinases, phosphatases, antibodies, antibody fragments, nucleic acids, e.g. antisense nucleic acids or siRNAs, ribozymes, and aptamers of the invention, as well as pharmaceutical compositions containing antibodies to the above biomolecules (e.g., antibodies to Spinl1 proteins, anti-idotypic antibodies) or immunoconjugates comprising such antibodies, or anticalins. It should be noted that methods for producing aptamers specific for proteins and nucleic acids are known. See, e.g., U.S. Pat. No. 5,840,867, U.S. Pat. No. 5,756,291, and U.S. Pat. No. 5,582,981. In addition, the agent may also be a chemical compound, e.g. a small molecule drug that may affect Spinl1 activity or expression directly. Furthermore, the agents may be biomolecules and chemical compounds, such as the ones listed above or below, that affect the interaction between Spinl1 and its physiologic ligands, including the cell membrane. The compositions are preferably suitable for internal use and include an effective amount of a pharmacologically active compound of the invention, alone or in combination, with one or more pharmaceutically acceptable carriers. The compounds are especially useful in that they have very low, if any toxicity.

In a preferred embodiment, the pharmaceutical composition is used for the prevention, amelioration, or treatment of obesity; obesity and diabetes, particularly type II diabetes; or diabetes, particularly type II diabetes.

In another preferred embodiment, the pharmaceutical composition is used for the prevention, amelioration, or treatment of obesity; obesity and diabetes, particularly type II diabetes; diabetes, particularly type II diabetes; chronic kidney disease; coronary atherosclerosis; anorexia nervosa; rheumatoid arthritis; osteoarthritis; osteoporosis; gastrointestinal diseases, in particular gastric disease, peptic ulcer, intestinal bowel disease, in particular Crohn's disease or ulcerative colitis; cardiac hypertrophy; abstructive sleep apnea; maculopathy, in particular age-related maculopathy (ARM) or degeneration (ARMD); and prostate cancer.

The agents mentioned above may be used in the pharmaceutical compositions of the invention combined with a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of REMINGTON'S PHARMACEUTICAL SCIENCES (18th ed.), Alfonso R. Gennaro, ed. (Mack Publishing Co., Easton, Pa. 1990), a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J., U.S.) or phosphate buffered saline (PBS). In all cases, the composition must be sterile and should be fluid to the extent that easy syringeability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifingal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

For instance, for oral administration in the form of a tablet or capsule (e.g., a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine.

Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.1 to 75%, preferably about 1 to 50%, of the active ingredient

The compounds of the invention can also be administered in such oral dosage forms as timed release and sustained release tablets or capsules, pills, powders, granules, elixers, tinctures, suspensions, syrups and emulsions.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated. Injectable compositions are preferably aqueous isotonic solutions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

The compounds of the present invention can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parental injectable administration is generally used for subcutaneous, intramuscular or intravenous injections and infusions. Additionally, one approach for parenteral administration employs the implantation of a slow-release or sustained-released system, which assures that a constant level of dosage is maintained, according to U.S. Pat. No. 3,710,795, incorporated herein by reference.

Furthermore, preferred compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would range from 0.1% to 15%, w/w or w/v.

For solid compositions, excipients include pharmaceutical grades of mannitol, lactose, starch, magnesium stearate, sodium saccharin, talcum, cellulose, glucose, sucrose, magnesium carbonate, and the like may be used. The active compound defined above, may be also formulated as suppositories using for example, polyalkylene glycols, for example, propylene glycol, as the carrier. In some embodiments, suppositories are advantageously prepared from fatty emulsions or suspensions.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564.

Compounds of the present invention may also be delivered by the use of monoclonal antibodies as individual carriers to which the compound molecules are coupled.

The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspnarnmidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, triethanolamine oleate, etc.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Oral dosages of the present invention, when used for the indicated effects, may be preferably provided in any form commonly used for oral dosage such as, for example, in scored tablets, time released capsules, liquid filled capsule, gels, powder or liquid forms. When provided in tablet or capsule form, the dosage per unit may be varied according to well known techniques. For example, individual dosages may contain 0.5, 1.0, 2.5, 5.0, 10.0, 15.0, 25.0, 50.0, 100.0, 250.0, 500.0 and 1000.0 mg of active ingredient. It is well known that daily dosage of a medication, such as a medication of this invention, may involve between one to ten or even more individual tables per day.

The compounds comprised in the pharmaceutical compositions of the present invention may be administered in a single daily dose, or the total daily dosage may be administered in divided doses of two, three or four times daily.

Any of the pharmaceutical compositions described above and claimed herein may contain 0.1-99%, preferably 1-70% (w/w or w/v) of the wild type Spinl1 polypeptide according to SEQ ID NO:3 or SEQ ID NO:7, the mutated Spinl1 protein according to the invention, or the fragments thereof, or of the nucleic acids, antibodies, and their various modified embodiments specifically described and claimed herein.

If desired, the pharmaceutical compositions can be provided with an adjuvant Adjuvants are discussed above. In some embodiments, adjuvants can be used to increase the immunological response which, depending on the host species, include Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Generally, animals are injected with antigen using several injections in a series, preferably including at least three booster injections.

In connection with the therapeutic applications described and claimed herein the compounds or agents of the present invention may be administered either alone or in combination with each other.

The compounds or agents of the present invention may furthermore be administered in conjunction with one or more other therapeutic compound(s), either in the same pharmaceutical composition, e.g., in a pharmaceutical composition as described and claimed herein, or as separate pharmaceutical compositions.

In one embodiment, the compounds or agents of the present invention are administered in conjunction with one or more compound(s) useful in the treatment of type II diabetes, e.g., one or more compound(s) selected from the group consisting of sulfonylureas (e.g., tolbutamide, chlorpropamide, tolazamide, azetohexamide, glyburide, glipizide, gliclazide, or glimepiride) or other insulin secretagogues (e.g., repaglinide, nateglinide), biguanides (e.g., metformin), or thiazolidines (e.g., rosiglitazone, pioglitazone), alpha-glucosidase inhibitors (e.g., acarbose, miglitol), and insulin or insulin analoga.

In another embodiment, the compounds or agents of the present invention are administered in conjunction with one or more compound(s) useful in the treatment of obesity, e.g., one or more lipase inhibitors, such as orlistat, or appetite suppressants, such as sibutramine.

If in connection with the above combination therapies the compounds or agents of the invention, or an agent or compound of the invention and the one or more other therapeutic compound(s), are administered in separate pharmaceutical compositions, they may be administered simultaneously or sequentially, the latter term including administration regimes where more than one administrations of the agent or compound of the invention are followed, or preceded, by more than one administrations of the one or more other compound(s).

In case of a sequential administration, the time interval between the administration of the agent or compound of the invention and the administration of the one or more other compound(s) is less than 1 week, more preferably less than 2 days, or even less than 1 day. Likewise preferred are embodiments where the time interval is less than 10 hours, preferably less than 5 hours, and more preferably less than 2 or even less than 1 hour.

More generally, the above combination therapies in accordance with the present invention preferably follow an administration regime where the administration of the agents or compounds of the invention, or the agent or compound of the invention in conjunction with the one or more other compounds, leads to a clinically detectable additive or synergistic effect which would not be observed if the same dosage of the agent or compound of the invention were administered alone.

Gene Therapy

A further aspect of the present invention is a method of gene therapy comprising delivering a DNA construct to cells in a human subject suffering from or known to be at risk of developing a condition associated with an alteration in fat metabolism. A DNA construct preferred in this regard comprises a sequence of an allele of the Spinl1 gene encoding the human Spinl1 protein of a human subject known not to have a medical condition associated with an alteration in fat metabolism, preferably a protein according to SEQ ID NO:7, or an allelic variant thereof, or a sequence encoding a protein having at least 63%, 65%, 70%, 75%, 80%, 85%, 90%, 95%, 98%, or 99% amino acid identity compared to the mouse Spinl1 or the human Spinl1 protein according to SEQ ID NO:3 and SEQ ID NO:7, respectively.

Also encompassed by the present invention is a method of gene therapy of the above kind wherein the DNA construct delivered to the cells of the human subject comprises a DNA sequence encoding the human Spinl1 protein according to SEQ ID NO:7 or an allelic variant thereof, or a human Spinl1 protein encoded by the Spinl1 gene of a human subject unaffected by or known not to be at risk of developing the above-mentioned condition, or a sequence encoding a protein having at least 63%, 65%, 70%, 75%, 80%, 85%, 90%, 95%, 98%, or 99% amino acid identity compared to the mouse Spinl1 or the human Spinl1 protein according to SEQ ID NO:3 and SEQ ID NO:7, respectively.

Furthermore encompassed are methods wherein the DNA construct comprises a DNA sequence encoding an antisense nucleic acid according to the invention, or an antisense nucleic acid comprising a nucleotide sequence which is complementary to an mRNA encoded by the Spinl gene of a human subject unaffected by or known not to be at risk of developing said condition.

Also encompassed are methods wherein the DNA construct comprises a DNA sequence encoding an siRNA as described and claimed herein.

The use of a DNA construct as described above in a method of treating a human subject suffering from, or known to be at risk of developing a medical condition associated with an alteration in fat metabolism, said method comprising delivering said DNA construct to at least some of the cells of said human subject is also encompassed within the present invention.

Method of Modulating Spinl1 Activity and Corresponding Uses

A further aspect of the present invention is a method of preventing, treating, or ameliorating a medical condition in a human subject associated with an alteration in fat metabolism, said method comprising administering to said human subject a pharmaceutical composition comprising an agent capable of modulating Spinl1 activity in said human subject.

One embodiment of said method is a method of gene therapy as described herein.

The agent capable of modulating Spinl1 activity may be one of the agents described and specifically claimed herein, e.g., one of the mutant Spinl1 proteins, nucleic acids and antibodies as previously defined.

It will be appreciated that such agents may, however, also be agents based on the wild type Spinl1 protein such as the wild type Spinl1 protein, or fragments thereof or fusion proteins comprising same, e.g., in situations where a reduced amount or activity of the endogenous Spinl1 is the cause of the above medical condition in the human subject. Accordingly, it will be appreciated that a wild type Spinl1 protein may advantageously be administered to a human subject suffering from such a condition, or a protein having a certain amino acid sequence identity and being effective in treating said medical conditions, wherein said effectiveness is determined by the same, or essentially the same, biological activity in an in vitro assays mentioned herein, e.g. in Example 18 (or a fragment or fusion of such protein). Proteins suitable in this regard may be readily determined e.g. with the help of these in vitro assays.

It will also be appreciated that antibodies against wild type Spinl1 protein or antisense nucleic acids or siRNAs encoding wild type Spinl1 may advantageously be administered, e.g., in situations where an excess of endogenous Spinl1 protein or Spinl1 activity is the cause of the medical condition in the human subject.

Another aspect of the present invention is a method of preventing, treating, or ameliorating a medical condition in a human subject associated with an alteration in plasma insulin level, or an alteration in insulin sensitivity, said method comprising administering to said human subject a pharmaceutical composition comprising an agent capable of modulating plasma insulin level or insulin sensitivity in said human subject, in particular an agent as defined and claimed in the context of the present invention.

The medical condition may be such that it is associated with insulin resistance, e.g., type 2 diabetes (also known as NIDDM), or obesitas, or that an individual affected by it would otherwise benefit from an increase in insulin sensitivity.

It is believed that in medical conditions associated with a decrease in plasma insulin level, or associated with insulin resistance, or in medical conditions wherein an individual affected thereby would benefit from an increase in insulin sensitivity, agents according to the present invention such as the antibodies directed against the Spinl1 wild type protein or fragments thereof, the Spinl1 mutant protein of the invention itself, or a fragment thereof, the antisense RNA, siRNA, aptamers, or anticalins directed against wild type Spinl1, Spinl1 antagonists, insulin activity or insulin sensitivity promoting agents, or a mutant Spinl1 protein displaying loss-of-function activity, will be therapeutically useful.

It is also believed that in medical conditions associated with an increased plasma insulin level, or in medical conditions wherein an individual affected thereby would benefit from a decreased insulin sensitivity, the Spinl1 wild type protein or a fragment thereof, Spinl1 agonists, insulin activity or insulin sensitivity inhibiting agents, or a mutant Spinl1 protein displaying gain-of-function activity, will be therapeutically useful.

Based on his/her common general knowledge and the assays provided by the present invention, for example the assays regarding the determination of plasma insulin levels and insulin activity or sensitivity, e.g., via the insulin resistance test described herein, the skilled person will in any event be readily able to determine whether a particular agent according to the present invention is suitable for the treatment of a particular medical condition associated with an alteration in fat metabolism or an alteration in plasma insulin level or insulin sensitivity.

Yet another aspect of the present invention is a method of preventing, treating, or ameliorating a medical condition in a human subject associated with an alteration in serum leptin level, or an alteration in leptin sensitivity, said method comprising administering to said human subject a pharmaceutical composition comprising an agent capable of modulating serum leptin level or leptin sensitivity in said human subject, in particular an agent as defined and claimed in the context of the present invention.

The medical condition may be such that it is associated with an increased serum leptin level, e.g., chronic kidney disease, or that an individual affected by it would benefit from a decrease in leptin sensitivity.

Alternatively the medical condition may be such that it is associated with a decreased serum leptin level, e.g., maculopathy, or that an individual affected by it would benefit from an increase in leptin sensitivity, e.g., obesity.

It is believed that in medical conditions associated with an increased serum leptin level, or in medical conditions wherein an individual affected thereby would benefit from a decrease in leptin sensitivity, the Spinl1 wild type protein or a fragment thereof, Spinl1 agonists, leptin activity or leptin sensitivity inhibiting agents, or a mutant Spinl1 protein displaying gain-of-function activity, will be therapeutically useful.

It is also believed that in medical conditions associated with a decreased serum leptin level, or in medical conditions wherein an individual affected thereby would benefit from an increased leptin sensitivity, agents according to the present invention such as the antibodies directed against the Spinl1 wild type protein or fragments thereof, the Spinl1 mutant protein of the invention itself, or a fragment thereof, the antisense RNA, siRNA, aptamers, or anticalins directed against wild type Spinl1, Spinl1 antagonists, leptin activity or leptin sensitivity promoting agents, or a mutant Spinl1 protein displaying loss-of-function activity, will be therapeutically useful.

Again, based on the assays provided by the present invention, for example the assays regarding the determination of serum leptin levels and leptin activity or sensitivity, e.g. via determination of STAT3 and/or AKT phosphorylation, the skilled person will in any event be readily able to determine whether a particular agent according to the present invention is suitable for the treatment of a particular medical condition associated with an alteration in fat metabolism or an alteration in serum leptin level or leptin sensitivity.

Assays and Diagnostics

The animals of the present invention present a phenotype, which is representative for many symptoms associated with an alteration in fat metabolism. Accordingly, the animal model of the present invention represents a particularly suitable model for the study of the molecular mechanisms and physiological processes associated with alterations in fat metabolism and medical conditions including obesity; obesity and diabetes, particularly type II diabetes; or diabetes, particularly type II diabetes; reduced activity or undesirable activity of endogenous Spinl 1; and reduced expression, reduced production or undesirable production of endogenous Spinl1. In particular, the animal model of the invention presents a phenotype characterized by an alteration in fat storage and/or an alteration in liver function.

The animals of the present invention can also be used to identify early diagnostic markers for diseases associated with an alteration in Spinl1 expression or activity. The term activity refers to an activity in both positive (=gain of function) and negative (=loss of function) ways. Surrogate markers, e.g. ribonucleic acids or proteins, may be identified by procedures comprising, e.g. ELISA, 2D-gel, protein microarrays or mass spectrophotometry, differential display, cDNA microarrays, DNA chips, quantitative PCR, RNAse protection assays, or Northern blotting. The test samples for the identification of said markers may derive from any organ or tissue, such as, e.g., blood samples. The test samples may further derive from different stages of the medical conditions. As used herein throughout the entire specification, a “test sample” refers to a biological sample obtained from a subject of interest. For example, a test sample might be a biological fluid (e.g., blood, plasma, serum), a cell sample, or a tissue sample.

The animal model of the present invention may further be used to monitor the activity of agents (e.g., an agonist, antagonist, peptidomimetic, protein, peptide, nucleic acid, small molecule, or other drug candidate) useful in the prevention or treatment of the above-mentioned medical conditions. The agent to be tested may be administered, e.g., as described herein in connection with the pharmaceutical compositions or methods, such as, e.g., gene therapy, to an animal of the present invention. The agent may be administered at different time points, doses and/or combinations of such agents. The activity of the agents might be monitored by their effects on the phenotype of the animal of the present invention, including but not restricted to histological parameters or food consumption as described, e.g. in Examples 3 and 4. Alternatively, the suitability of the agents for therapeutic use might be determined by monitoring differences in the metabolism of the agent, which may lead to severe toxicity or by altering the relation between dose and blood concentration of the pharmacologically active drug. Moreover, the determination of the pharmacogenomics of an individual permits the selection of effective agents for prophylactic or therapeutic treatments based on the individual's genotype. Such pharmacogenomics can further be used to determine appropriate dosages and therapeutic regimens.

Furthermore, the animals of the present invention may be used for the dissection of the molecular mechanisms of the Spinl1 pathway, e.g. for the identification of receptors or downstream genes or proteins regulated by Spinl1 activity. To identify such mechanisms, e.g. ELISA, 2D-gel, protein microarrays or mass spectrophotometry, differential display, cDNA microarrays, DNA chips, quantitative PCR, RNAse protection assays, or Northern blotting may be employed. The test samples for the identification of said markers may derive from any organ or tissue, such as, e.g., blood samples. The test samples may derive further derive from different stages of the medical conditions.

The methods described herein might be utilized to identify subjects having or being at risk of developing a medical condition associated with aberrant Spinl1 expression or activity, e.g. by the diagnostic assays described herein.

Alternatively, the assays might be used to identify a subject having or being at risk for developing a medical condition associated with an alteration in fat metabolism. Thus, the invention provides a method in which a test sample, e.g. protein or nucleic acid sample, is obtained from a subject, particularly a human subject, and wherein the presence of a mutation in the Spinl1 protein or nucleic acid specific for Spinl1, or an increase or reduction in expression of the Spinl1 protein or the nucleic acid specific for Spinl1 is indicative for a subject having or being at risk of developing a medical condition associated with altered Spinl1 expression or activity.

The methods described herein may further by used to determine Spinl1 activity or expression in test samples derived from patients, particularly samples from blood, serum or plasma. The test samples may be analyzed directly or after extraction, isolation and/or purification by standard methods.

The diagnostic methods may further be used to identify a modified Spinl1, wherein the modification is associated with the replacement of an amino acid at a position corresponding to position 108 in the amino acid sequence shown in SEQ ID NO:7. In another embodiment of the invention, the diagnostic method may comprise the identification of a mutant Spinl1, wherein the mutation is associated with the insertion of additional amino acids normally not present in the amino acid sequences of SEQ ID NO:3 or SEQ ID NO:7 of mouse or human Spinl1. Methods of identifying the mutant Spinl1 protein may include any methods known in the art which are able to identify altered conformational properties of the mutant Spinl1 protein compared to those of the wild type Spinl1, e.g. by the specific recognition of the mutant protein by other proteins, particularly antibodies, e.g. antibodies directed to an epitope only present in mutant Spinl1 as compared to wild type Spinl1 or by comparing individual or combined patterns of wild type and mutant protein digestion by known proteases or chemicals. In an additional, related embodiment, the method exploits the failure of another protein to recognize the mutant Spinl1 protein.

The invention will be further described in the following examples. The examples are offered for illustrative purposes only, and are not intended to limit the scope of the present invention in any way.

### EXAMPLES

### Example 1

### ENU (Ethyl-nitroso-urea) Treatment to Produce Mutagenized Animals

To produce mutants, C3HeB/FeJ male mice (The Jackson Laboratory, Bar Harbor Me., U.S.) were injected intraperitoneally three times (weekly intervals between 8-10 weeks of age) with ethyl-nitroso-urea (ENU) (Serva Electrophoresis GmbH, Heidelberg, Germany) at a dosage of 90 mg/kg body weight The injected male mice were regularly mated to wild type C3HeB/FeJ female partners fifty days after the last injection. The resultant F1 progeny (up to 100 offspring) were then analyzed for dominant phenotypes.

Generation of F3 Progeny

F3 progeny were generated as described below. All breeding partners were older than 8 weeks (56 days); preferably females were between 8-12 weeks old and males were between 8-16 weeks old.

Production of F1-animals (db1)

Each ENU-male produced as described above was used to generate more than 30 male and 30 female pups, which were interbred as described below.

Production of F2-animals (rf1)

Twenty matings were set up as follows: (1 male F1(db1)×1 female F1(db1) to produce 20 pedigrees. The animals of one breeding pair are pups of different ENU-animals (mating type: rf1). At the age of 8 weeks of rf1 animals, a single F2 (1 male)×F2 (1 female)−breeding per pedigree was started (mating type: rbs). From each rbs-breeding, at least 15 offspring were produced. Rf1-females were kept until the youngest rbs animals had been screened (age=160 days). Rf1-males were sacrified and frozen after the offspring had reached the number of 15 animals. F3 animals went into the primary screen.

We performed a series of tests on F3 animals as a primary screen to identify relevant phenotypes. For this invention, blood sampling and analysis provided information to identify an aberrant phenotype within the F3 population.

### Example 2

### Physiological Characteristics of the Mutant Animals

For blood analysis animals were starved over night. Animals were anaesthetized by ether. 500-600 μl blood per animal was taken retro-orbitally by a heparinized capillary and collected in heparin tubes. Blood plasma was separated by centrifugation. The following sixteen plasma parameters were measured with a Hitachi 912 using the recommended reagents according to the manufacturers instructions (Roche Diagnostics, Mannheim, Germany): calcium, creatinine, phosphate, glutamic-oxaloacetic tsaminase (GOT), glutamate pyruvate transaminase (GPT), lactate dehydrogenase (LDH), cholinesterase (CHE), triglycerides, glucose, total protein, urea, alkaline phosphatase (ALP), cholesterol (CHOL), high density lipoprotein (HDL), Low density Lipoprotein (LDL) and lactate (LACT). Values are considered to be abnormal if they are beyond the 99 or 1 percentile, respectively.

The clinical chemistry of animals homozygous for the Spinl1 mutation was abnormal for nine of the sixteen parameters analyzed, shown in FIG. 1. This includes elevation of ALP, GOT, GPT, LDH, CHE, CHOL, HDL, LDL and reduction of lactate.

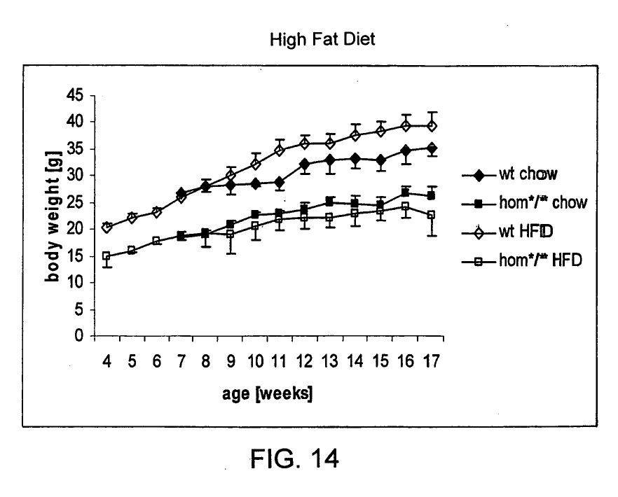

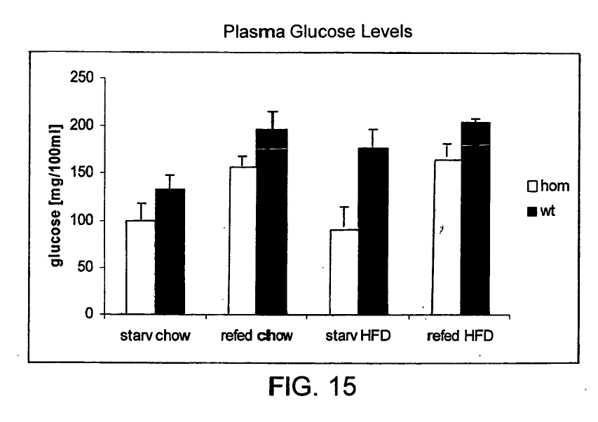

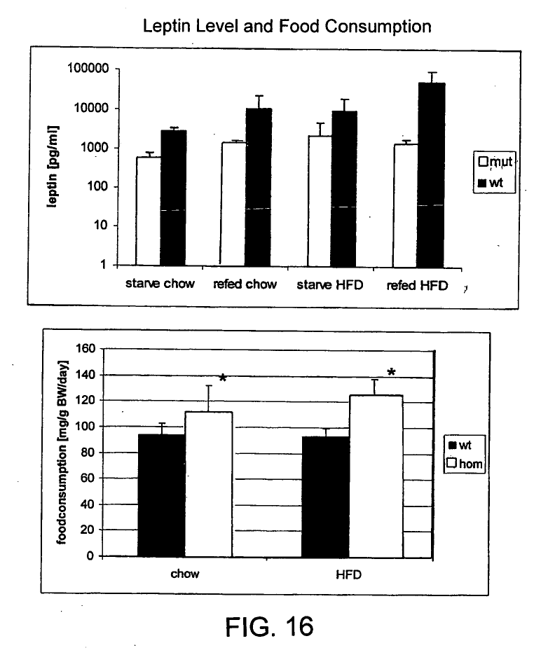

Elevated blood serum levels for cholesterol (CHOL), cholinesterase (CHE), high density lipoprotein (HDL), and low density lipoprotein (LDL) are indicative for an altered cholesterol metabolism. Elevated blood serum levels observed for liver enzymes, such as alkaline phosphatase (ALP), glutamic-axaloacetic transaminase (GOT), glutamate pyruvate transaminase (GPT), and lactate dehydrogenase (LDH) are indicative for a disturbed liver function. An alteration of serum level was observed for lactate. Moreover, mutant animals displayed a significantly lower serum level of leptin, a cytokine produced by adipocytes. Leptin is known to be involved in regulation of energy intake and expenditure. Reduced leptin levels have been detected under different experimental conditions of food consumption, like normal food diet and high fat diet (see FIG. 16A). Despite reduced leptin levels the food intake was only slightly elevated in Spinl1 mice (see FIG. 16B). In addition, Spinl1 mice are hypoglycaemic under these feeding conditions: plasma glucose levels in affected mice (hom) are always lower compared to wild type controls (wt) (see FIG. 15). Feeding of high fat diet (HFD) to mutant animals did neither induce obesity (see FIG. 14) nor diabetes. These data indicate a Spinl1 protein function in the leptin pathway. The penetrancy of this phenotype was 100%.

Moreover, the animals displayed a thriving deficit, which manifests in reduced weight and in reduced body length, when compared to wild type littermates (body weight was observed during a time course between day 35 and day 91 after birth (FIG. 2a, b). Body length was measured between 132 and 148 days after birth (FIG. 3a).

Body composition was analyzed using a pDEXA instrument (Nordland pDEXA Sabre, Stratec Medizintechnik, Pforzheim, Germany) according to the manufacturers instructions. The method relies on different absorption characteristics of fat, muscle and bones in different spectral bands. For the pDEXA investigation, mice were anaesthetize by intraperitoneal injection of 5 μl/g bodyweight of 0.5% Keta+min (WDT, Garbsen, Germany) and 0.2% Rompun (Bayer AG, Leverkusen, Germany) in 0.9% NaCl solution (B. Braun, Melsungen, Germany). The anaesthetized animals were fixed on the scanning area The investigation revealed a dramatic and disproportional reduction in body fat content (FIG. 3b).

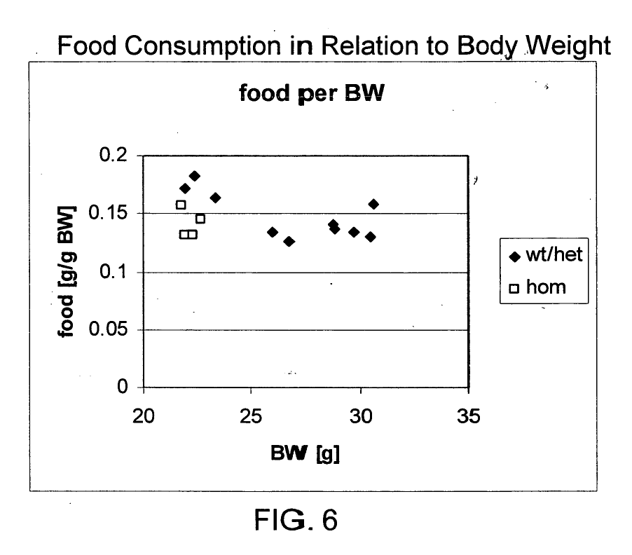

Food consumption of affected and wild type animals was analyzed (see Example 4). Although body weight was reduced, food consumption was not significantly different in affected homozygous mice, compared to wild type and heterozygous mice (FIG. 6).

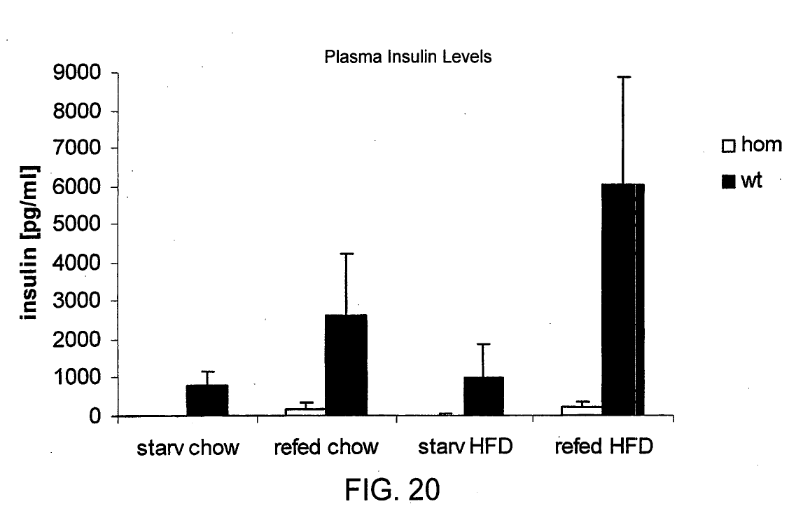

Plasma insulin levels of affected and wild type mice were analyzed using a Mouse Insulin ELISA Kit (EIA-3440, DRG Diagnostics, Marburg, Germany) according to the manufacturers instructions. Significantly reduced insulin levels were detected under different experimental conditions of food consumption, like normal food diet and high fat diet, after overnight starving or refeeding (see FIG. 20).

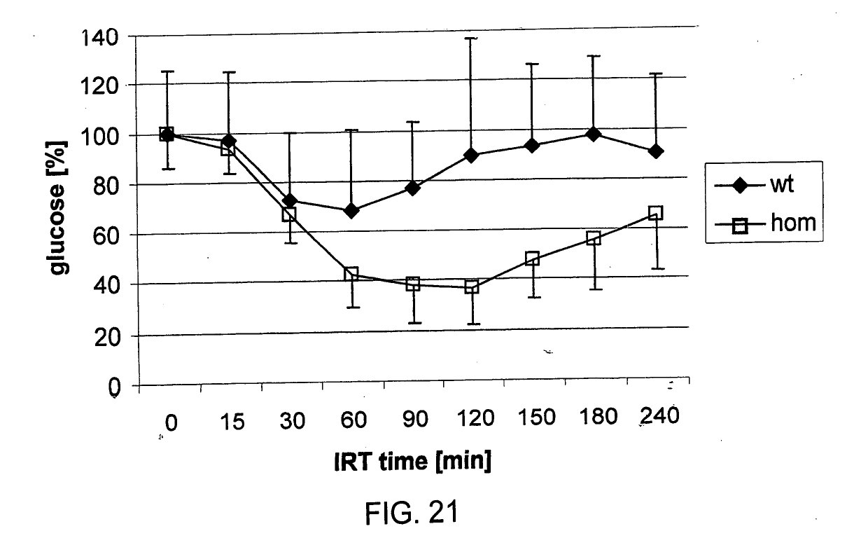

Additionally, an Insulin Resistance Test (IRT) was performed with affected and wild type mice (FIG. 21, Example 24). Data reveal a hyperreactive and prolonged response to insulin injection in Spinl1 affected mice, indicating an elevated insulin sensitivity of Spinl1 affected mice (see FIG. 21).

### Example 3

### Necroscopy and Histology of the Mutant Animals

Macroscopic examination of sacrificed animals corroborated the pDEXA findings. Subcutaneous, intraperitoneal and gonadal fat was virtually absent.

Fixation, processing and staining was performed according to histological standard procedures.

Microscopic examinations of the kidneys revealed the absence of intracellular fat vacuoles (arrows in FIG. 4A, haematoxylin & eosin stains of 5 μm paraffin sections). No further abnormalities were observed in the kidney histology. The perirenal fat pad contained white adipose tissue with interspersed islets of brown adipose tissue (arrowheads in FIG. 4b). The white adipocytes were reduced in size compared to wild type (arrows in FIG. 4b). This points to a reduced fat storage with otherwise undisturbed cellular function.

Histological examination of liver tissue revealed most conspicuous microscopical changes. Wild type (wt) animal liver sections, stained with haematoxylin & eosin, displayed a homogeneous network-like cytoplasmic pattern in hepatocytes. Unstained granular structures are due to glycogen and lipid droplets which have been dissolved during the processing (FIG. 5a). Hepatocytes of affected animals showed a heterogeneous distribution of cytoplasmic staining. The staining was more intense at the sinusoidal pole and only pale adjacent to the biliary pole. The network-like structure of the wt cytoplasm was absent.

In affected animals increased numbers of apoptotic hepatocytes were present (councilman bodies, arrows in FIG. 5a).

Transmission electron microscopic (TEM) analysis of liver tissue was performed. Tissue was fixed with 4% glutaraldehyde, postfixed with 1% osmiumtetroxide, dehydrated, and embedded in EPON 813. Ultrathin sections were stained with uranylacetate and lead citrate and examined with an electron microscope. TEM analysis revealed the presence of electron dense material in the hepatocytes of affected animals. This material is often arranged in concentric structures known as lamellar bodies of myelin-like structures. Similar structures were observed in Kupfer cells. Mitochondria, rough endoplasmic reticulum, ribosomes and the Golgi apparatus showed no differences compared with wt animals. These organelles were concentrated at the sinusoidal pole and the electron dense material adjacent to the bile capillary. The amount of stored glycogen is in the normal range, whereas fat vacuoles are largely reduced in the affected animals (FIG. 5b).

The histological differences observed in the liver are in accordance with the abnormalities detected by clinical chemistry (Example 2).

### Example 4

### Measuring of Food Consumption

For measurement of food consumption animals were housed in separate cages (one animal per cage). After 3 days of adaptation a defined amount of food was provided and weighed 24 hrs later. Food pellets distributed within the cage were collected from the bedding and were also weighed. The difference between the provided amount and the remaining plus dissipated amount is the food consumed. This measurement was repeated for at least five days and the mean daily food consumption was calculated as the average of these values. Food consumption is slightly elevated in homozygous Spinl1 mice, compared to wild type controls, as seen in FIG. 16B and in FIG. 6.

### Example 5

### Mapping and Cloning of the Mutation in the Mutant Animals of the Present Invention

1. Generation of F5 Outcross Mice for Subsequent Chromosome Mapping

We generated F5 progeny by breeding a phenotypically identified F3 mutant with C57B1/6 mice for generation of F4 outcross mice, and subsequent intercrossing of the F4 progeny to produce the F5 generation. The F5 generation was tested for the presence of abnormal parameters of clinical chemistry. The F5 outcross mice were used to locate the causative ENU mutation.

2. DNA Isolation from Rodent Tails

Mouse genomic DNA was purified from 1 cm long pieces of mice tail by using the “DNeasy 96 Tissue Kit” (Qiagen, Hilden, Germany) according to the manufacturer's protocol.

3. Macromapping

In F5 outcross mice allele frequencies of C57B/16 versus C3H alleles are 1:1 in average, following Mendelian rules of inheritance. Arrangement in groups of phenotypic positive and phenotypic negative mice alters this ratio only at marker positions in the vicinity of the phenotype causing the mutation, driving it towards 0:1 in the phenotypic positive group and 1:0 in the phenotypic negative group. Allele frequency analysis of distributed genome covering markers (e.g., SSLP, SNP) in a group of phenotypic positive F5 outcross mice indicate the site of the mutation by a C3H:C57B1/6 ratio above 3.

For the affected mice we searched for a chromosomal locus with increased allele frequency for single nucleotide polymorphisms (SNPs) representing the C3H strain. Markers in this analysis were about 90 SNPs polymorphic between C3H and C57B1/6 strains equally distributed over the 19 autosomal mouse chromosomes. Analysis was done on pooled tail DNA samples of F5 outcross mice phenotypical affected with the first step: competitive PCR; followed by second step: SNP allele frequency measurement from the PCR product mix by Pyrosequencing technology (PSQ 96 system; http://www.pyrosequencing.com/pages/applications.html).

Pooled tail DNA (1 ml pooled DNA, 10 μg/ml: 10 μg/9 mice=1.11 μg/mouse, DNA concentration measurement by UV-spectrophotometer) was distributed in a 96-well plate with pre-deposited SNP marker PCR primers (one SNP/well). A standard PCR reaction was performed (50 μl vol.). One of both SNP primers was biotinylated, which is necessary for the subsequent single strand PCR product purification in the Pyrosequencing procedure. Purification of a single stranded (ss) PCR product and short range sequencing the SNP positions on the ss PCR product was performed according to the instructions supplied with the Pyrosequencing kit (PSQ 96 SNP Reagent Kit, 5×96). The resulting peaks at the polymorphic bp positions of the SNP sequence correlate with the amount this allele had in the original DNA pool and were exported from the PSQ 96 databank and processed into an Excel macro.

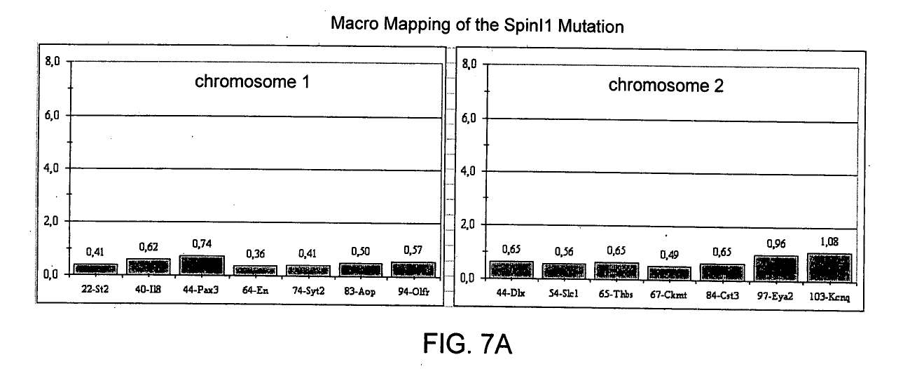

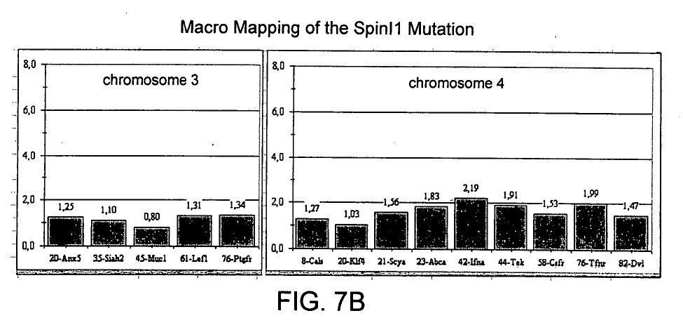

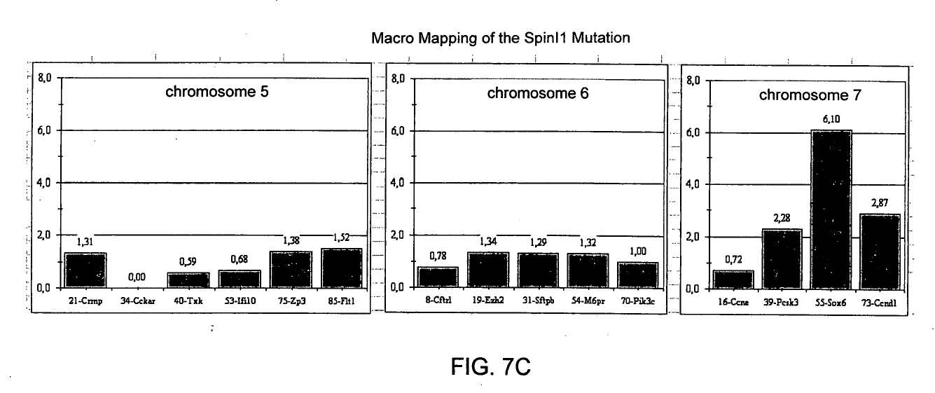

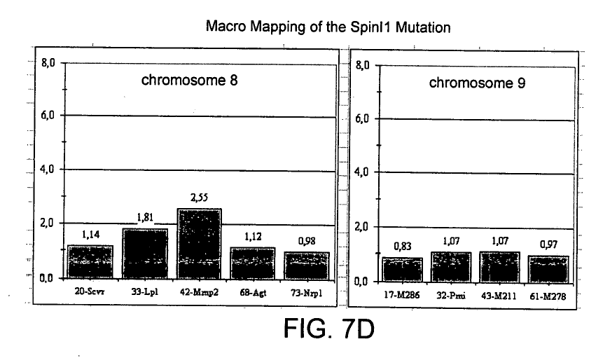

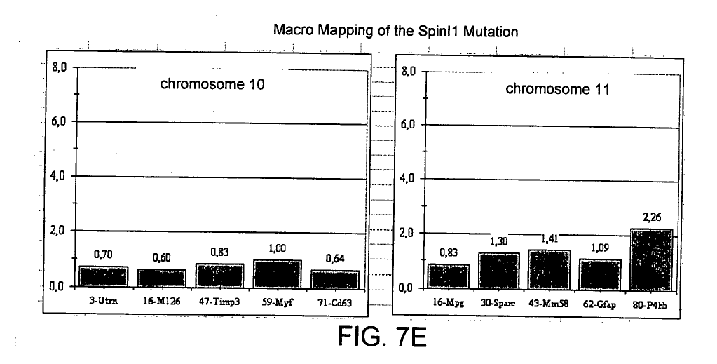

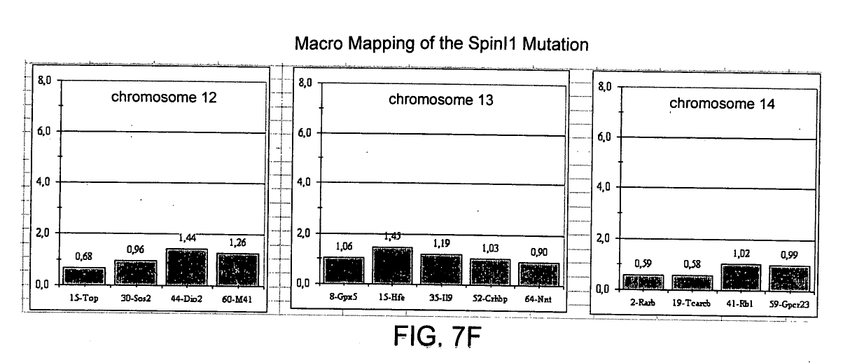

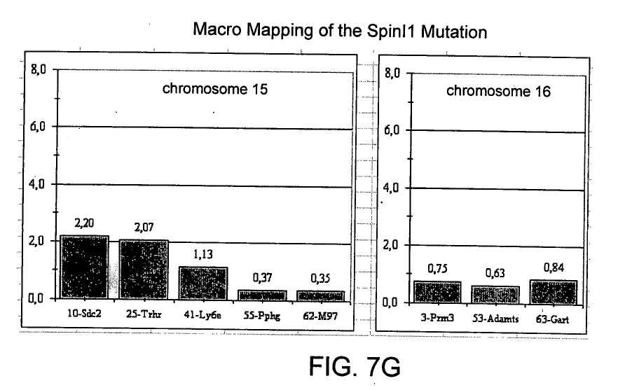

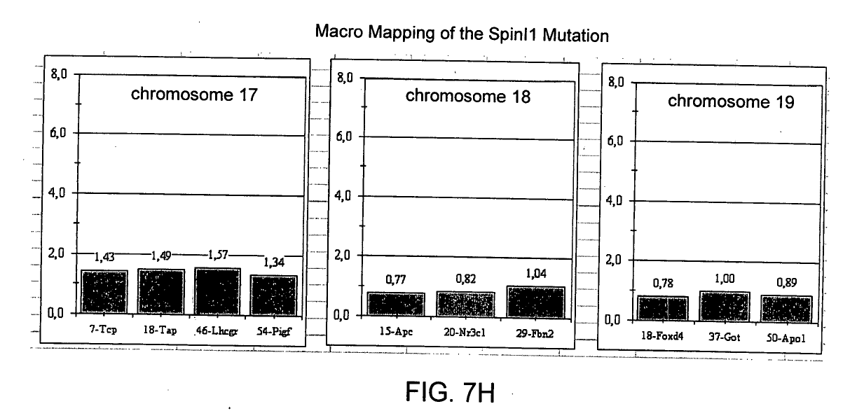

The Excel macro calculated the C3H/BL6-peakhight ratio at every SNP position according to the formula: (peakhight^{C3H}/peakhight^{BL6})/constant^{individualSNP}. Constant^{individualSNP} serves to improve C3H/BL6-peakhight ratio comparability among different SNP positions and is an average value for peakhight^{C3H}/peakhight^{BL6} of a heterozygous C3H/C57B1/6 mouse (F1 outcross mouse). This value was determined experimentally afore for every individual SNP from nine (triplicates on three days) measurements and is expected to be close to 1. Finally the Excel macro delivered a graphical output from the calculated B16/C3H-peakhight ratios (FIG. 7) in which regions with values above 3 indicate the chromosomal position of the mutation.

DNA pool analysis of 9 phenotypically affected animals showed high values above 3 for chromosome 7 and assigned the mutation to chromosome 7, 39-73 cM.

4. Fine Mapping

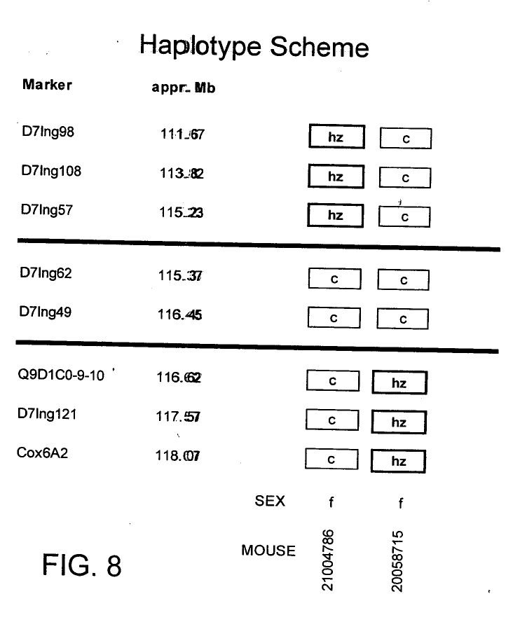

The initial mapping was confirmed on single mouse level haplotype analysis of a total of 641 F5 outcross affected mice using SNP or microsatellite markers located in the critical region on chromosome 7. The candidate region mapping was refined, based on mice that carry chromosomal break points in the respective region. Finally the analysis narrowed the location of the mutation to an interval of approximately 1.39 Mbp between the microsatellite marker D71 ng57 (SEQ ID NO:9, PCR amplification was performed with primers SEQ ID NO:10 and SEQ ID NO:11) and microsatellite marker Q9D1C0-9-10 (SEQ ID NO:12, PCR amplification was performed with primers SEQ ID NO:13 and SEQ ID NO:14). This was evident because the phenotypically affected mouse 21004786 excluded the region proximal of D71 ng57, while phenotypically affected mouse 20058715 excluded the region distal of Q9D1C0-9-10 (FIG. 8). This led to the conclusion that (a) gene(s) located entirely or partially between both markers could contain the mutation.

The genomic interval between markers D71 ng57 and Q9D1C0-9-10 was scanned for genes by a detailed analysis of public mouse and human genome databases. Several annotated mouse genes were recorded within this region. Of these, the identified Spinl1 gene was considered one of the relevant candidate genes and was further analyzed.

5. PCR Amplification and Sequencing of Mouse Spinl1 Gene

The genomic structure, the precise location of Spinl1 exons, and the putative full length cDNA (part of SEQ ID NO:1) containing the open reading frame coding for the Spinl1 protein (SEQ ID NO:3), the polyadenylation signal, and the polyA tail was deduced from public mouse Spinl1 cDNA (Genbank accession number AF212372) and from genomic mouse DNA data (Ensemble, February 2002 freeze of the mouse assembly). The same was done for human Spinl1 (Genbank accession number AF212371; SEQ ID NO:5). Mouse Spinl1 comprises 12 exons which very closely resemble those of human Spinl1 with respect to size, sequence, genomic context, and chromosomal exon distribution.

Genomic DNA fragments of the murine Spinl1 gene were obtained by PCR using BioTherm-DNA-polymerase (GeneCraft, Germany) according to the manufacturer's protocol. Oligonucleotide primers were designed using a publicly available primer design program (Primer3, www.genome.wo.mit.edu) to generate a series of oligonucleotide primers specific for Spinl1 genomic sequences. Primers used for amplification are shown in SEQ ID NO:15 to SEQ ID NO:26. Primers of SEQ ID NO:15, 16, 17, and 18, were used to amplify exon 1 and exon 2, primers of SEQ ID NO:19 and 20 were used to amplify exon 3, primers of SEQ ID NO:21 and 22 were used to amplify exons 4 and 5, primers of SEQ ID NO:23 and 24 were used to amplify exon 6, primers of SEQ ID NO:25 and 26 were used to amplify exon 7, primers of SEQ ID NO:27 and 28 were used to amplify exon 8, primers of SEQ ID NO:29 and 30 were used to amplify exons 9 and 10, primers of SEQ ID NO:31 and 32 were used to amplify exon 11, and primers of SEQ ID NO:33 and 34 were used to amplify exon 12. PCR amplified products were purified using the QIAquick PCR Purification Kit (Qiagen, Hilden, Germany) according to the manufacturer's protocol. PCR products were sequenced using forward/reverse PCR primers and the “Big Dye” thermal cycle sequencing Kit (ABI PRISM, Applied Biosystems, Foster City, Calif., U.S.). The reaction products were analyzed on an ABI 3700 DNA sequencing device.

6. Sequence Analysis

The sequences were edited manually and different sequence fragments were assembled into one contiguous sequence by the software Sequencer version 4.0.5. (Gene Codes Corp., Ann Arbor Mich., U.S.A.). We sequenced the Spinl1 gene in phenotypically affected, homozygous F2 outcross mice as well as phenotypically non-affected, heterozygous mice. In both cases, C3H and C57B1/6 mice sequences were used as controls. The sequencing results showed that exons 1-2 and exons 4-12 were free of any mutation. However, a single bp exchange was detected in exon 3, replacing a Thymine (underlined T in the wild type exon 3 sequence, SEQ ID NO:35) to Cytosine (underlined C in the mutated exon 3 sequence, SEQ ID NO:36). The mutation was confirmed in all phenotypically affected mice tested. Sequencing the coding region of other genes in the candidate region showed that those were free of any additional mutations.

As a consequence of the mutation the codon TAC is changed to CAC and the aromatic Tyrosine (Y) amino acid residue at position 108 of the wild type protein (SEQ ID NO:3) is replaced by the basic Histidine (H) amino acid residue in the mutated protein (SEQ ID NO:4).

### Example 6

### Characterization of Mouse Spinl1 Protein

Mouse Spinl1 is a membrane protein with an overall structure of a transmembrane transporter with highest similarity to glucose transporters. Twelve transmembrane domains (TM1 to TM12) are predicted for mouse spinl1, according to Ensembl Peptide ID ENSMUSP00000032994 (Ensembl Gene ID ENSMUSG00000030741), and as summarized below.

Transmembrane Domains of Mouse Spinl1 like protein

| Transmembrane | | | |
|---|---|---|---|
| Domain (TM) | start | end | |
| TM 1 | 60 | 82 | |
| TM2 | 97 | 119 | (site of 108Y to 108H exchange) |
| TM3 | 126 | 145 | |
| TM4 | 158 | 180 | |
| TM5 | 187 | 206 | |
| TM6 | 216 | 238 | |
| TM7 | 272 | 294 | |
| TM8 | 322 | 344 | |
| TM9 | 357 | 379 | |
| TM10 | 389 | 411 | |
| TM11 | 423 | 442 | |
| TM12 | 462 | 484 | |

The mutation detected in transmembrane domain 2 (amino acid 108Y to 108H) interferes with the protein function, thus causing changes of the fat content of the animal, of its cholesterol and liver metabolism.

### Example 7

### Identification of Orthologue Proteins of Mouse Spinl1 Protein

In a protein homology search with mouse Spinl1 protein sequences, using the NCBI BLAST and the Ensembl BLAST, several orthologue proteins from other species were identified, comprising Spinl1 from human (Homo sapiens, Genbank Accession No. AAG43830), zebrafish (Danio rerio, Genbank Accession No. NP_{—}70594), rat (Rattus norvegus, Ensembl Peptide ID ENSRNP00000024185), and fugu (Takafugus rubiens, Ensembl Peptide ID00000128970 plus amino acid sequences annotated from Ensembl genome scaffold_{—}7549).

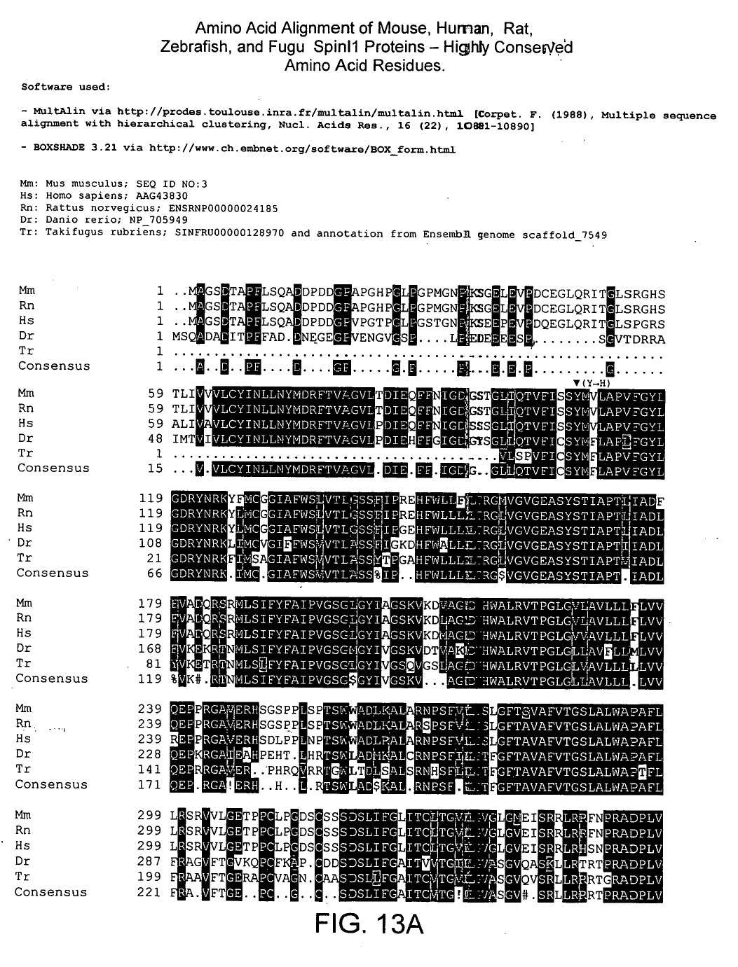

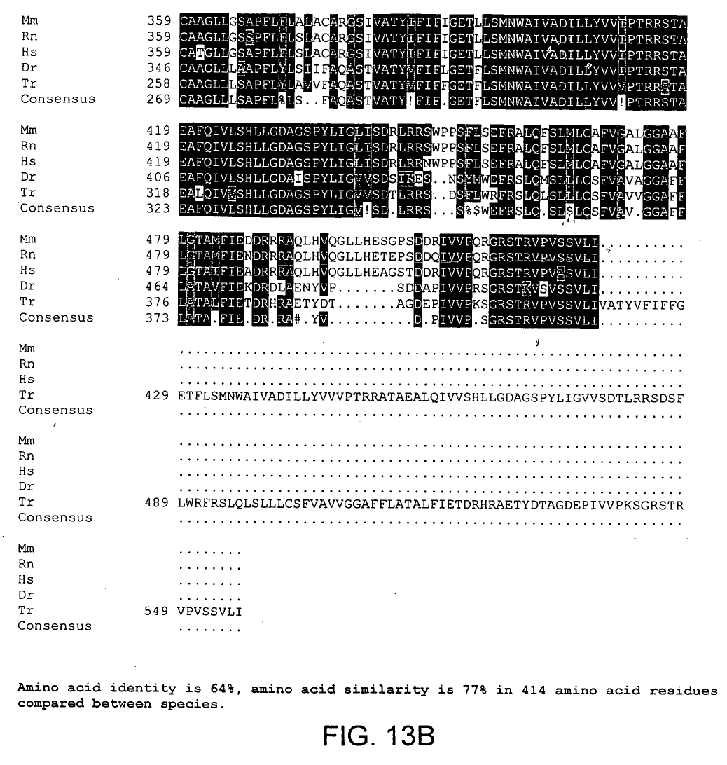

A ClustalW-Alignment of those proteins indicates that position 108Y of mouse Spinl1 is conserved between the Spinl1 orthologues from different vertebrate species, at equivalent positions (see FIG. 13).

### Example 8

### Comparison of Mouse and Human Spinl1 Proteins

Both, mouse and human Spinl1 genes, comprising twelve exons each, encode proteins of 528 amino acids in size. According to public protein data (Ensembl Peptide ID ENSMUSP000032994 for mouse Spinl1, and Ensembl Peptide ID ENSMUSP00000032994 for human Spinl1), the mouse protein bears twelve transmembrane domains (TM1 to TM12), whereas the human protein bears eleven transmembrane domains (TM1 to TM11). TM1 to TM11 of human Spinl1 correspond to mouse TM2 to TM12, with almost identical TM size and almost identical amino acid sequence. The amino acid positions of the corresponding TMs are:
97-119 (1M2 mouse and TM1 human)
126-145 (TM3 mouse) and 126-148 (TM2 human)
158-180 (TM4 mouse and TM3 human)
187-206 (TM5 mouse and TM4 human)
216-238 (TM6 mouse and TM5 human)
272-294 (TM7 mouse and TM6 human)
322-344 (TM8 mouse and TM7 human)
357-379 (TM9 mouse) and 356-378411 (TM8 human)
389411 (TM10 mouse) and 388-419 (TM9 human)
423-442 (TM11 mouse and TM10 human)
462-484 (TM12 mouse and TM11 human)

The amino acid identity between both proteins is 93%, the amino acid similarity is 94.7% (see FIG. 10).

Human Spinl1 is described in WO 02/055701. It is suggested in that reference to be a member of the sugar transporter family. No functional data are provided therein.

### Example 9

### Method for Production of the Mutant Animals of the Present Invention by Gene Targeting Technology

The construction of a recombinant targeting vector to insert a point mutation in exon 3 of the mouse Spinl1 gene may be performed according to well known techniques. For example the Lambda-KO-Sfi system of Nehls and Wattler, WO 01/75127 (A2).

1. Vector Construction

In a first step, a 1.5 kbp genomic DNA fragment is PCR amplified, representing the left arm of homology of the targeting vector to be constructed. After subsequent subcloning of the PCR fragment into a plasmid vector, i.e. pCR 2.1-TOPO (K4500-01, Invitrogen, Carlsbad, Calif., USA), according to the manufacturer's instructions, plasmid DNA, bearing the correct Spinl1 insert is subject to site-directed mutagenesis, using a QuickChange. Site-Directed Mutagenesis Kit (200518, Stratagene, La Jolla, Calif., USA), as outlined in the manufacturer's instructions. In brief, the plasmid vector (parental DNA template) and two oligonucleotide primers, each primer complementary to opposite strands of the vector insert and containing the desired point mutation (exon 3, position 665 of Spill cDNA), are denatured and subjected to PCR amplification with a proof-reading DNA polymerase (Pfu Turbo), provided in the kit. Using the non-strand displacing action of Pfu Turbo DNA polymerase, mutagenic primers are incorporated and extended, resulting in nicked circular DNA strands. In a restriction digest with DpnI, only the methylated parental DNA template is susceptible to DpnI digestion. After transformation in XL1-Blue supercompetent cells, provided with the kit, nicks in the mutated (point mutation) plasmid DNA are repaired Mutation positive colonies are selected and plasmid DNA is isolated, according to the manufacturer's instructions (Stratagene, La Jolla, Calif., USA).

Plasmid DNA, bearing the point mutation in exon 3, as described in the present invention, is subject to PCR amplification with primers, bearing SfiC and SfiA sequence overhangs, respectively, as described in the published patent application WO 01/75127 (A2). The PCR fragment, representing the left arm of homology is further processed, as described in the afore-mentioned patent application. The vector described in WO 01/75127 (A2), includes a linear lambda vector (lambda-KO-Sfi) that comprises a stuffer fragment, an E. coli origin of replication, an antibiotic resistance gene for bacteria selection, two negative selection markers suitable for use in mammalian cells, and LoxP sequences for cre-recombinase mediated conversion of linear lambda phages into high copy plasmids. In a final lambda targeting vector, the stuffer fragment is replaced by Sfi A, B, C, D ligation of the left arm of homology (bearing the Spinal point mutation in exon 3), an ES cell selection cassette, and a right arm of homology, as described in the afore-mentioned patent application. In-vitro packaging of the ligation products, plating of a phage library, plasmid conversion, and DNA isolation of the homologous recombination plasmid vector is performed according to standard procedures, known by persons skilled in the art.

2. ES cell Transformation and Mice Production

Targeting vectors containing the point mutation are used for mouse ES cell transformation and for producing chimeric mice by blastocyst injection and transfer using standard methodology, well known in the art. The chimeras are bred to wild type mice to determine germline transmission. Heterozygotes and subsequently homozygotes are generated according to well known techniques.

### Example 10

### Method for the Production of Transgenic Non-Human Animals Carrying a Transgene of Spinl1, Produced by Gene Targeting Technology

Transgenic mice carrying a mammalian Spinl1 transgene are generated by either using the embryonic stem cell method, or the pronucleus method, both of them well-known methods in the art; preferably using the method of Nehis and Wattler, as described in WO 01/75127. For transgenic methods see also US patents U.S. Pat. No. 6,436,701, U.S. Pat. No. 6,018,097, U.S. Pat. No. 5,942,435, U.S. Pat. No. 5,824,837, U.S. Pat. No. 5,731,489, and U.S. Pat. No. 5,523,226.

A transgene construct may contain a liver specific promoter, like the promoter of the mouse albumin gene.

### Example 11

### Tissue Specific Expression of Mouse Spinl1 RNA, Analysis by Northern Hybridization

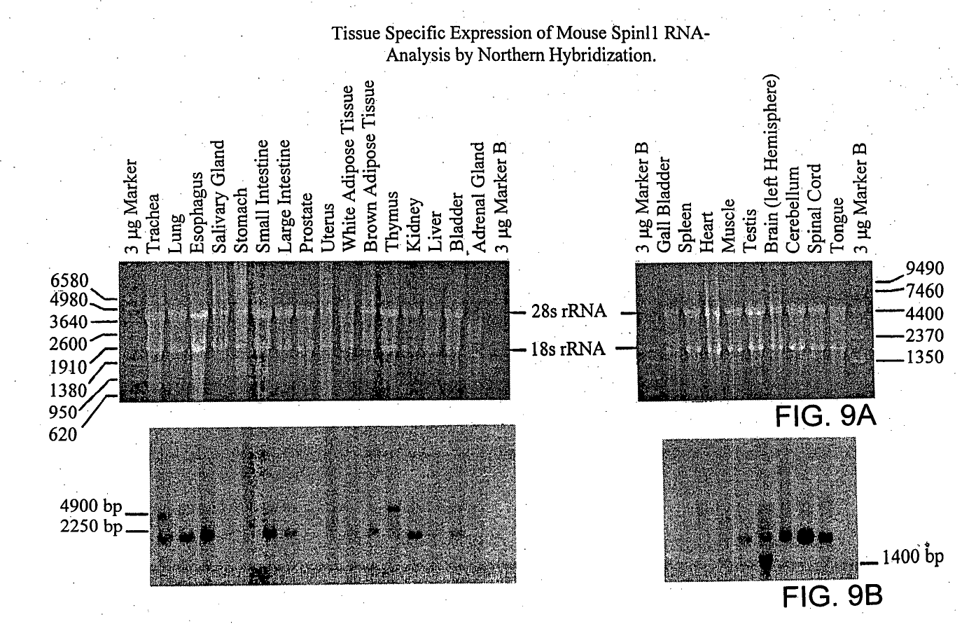

Northern hybridization of fresh isolated total RNAs from several mouse tissues was carried out using a mouse Spinl1 specific DNA-probe. The probe was generated by radiolabeling a purified and sequenced PCR product (SEQ ID NO:39), generated using primers SEQ ID NO:37 and SEQ ID NO:38. The probe was 1032 bp in length and included sequences coding for TM6 to TM12. Multiple tissue Northern blots were prepared according to standard methods known in the art, for example described in Sambrook et al., 1989, “Molecular Cloning, A Laboratory Manual”, Cold Spring Harbor Press, New York, USA. The blots carried each 15 micrograms of total RNA per lane. The tissues represented at the Multiple Tissue Northern Blots are as follows: trachea, lung, esophagus, salivary gland, stomach, small intestine, large intestine, prostate, uterus, white adipose tissue, brown adipose tissue, thymus, kidney, liver, bladder, adrenal gland, gall bladder, spleen, heart, skeletal muscle, testis, brain (left hemisphere), cerebellum, spinal cord, and tongue. Each 3 microgram of RNA size markers (type A and type B) were loaded as size reference. RNA probes were mixed with ethidiumbromid as transilluminescence before eletrophoresis through an agarose gel. Before blotting, the agarose gels were photographed at an UV transilluminator in order to verify the RNA quality by examining the 28S- and 18S-rRNA and to detect the RNA size marker bands (see FIG. 9A) Pre-hybridization was done for 30 minutes and hybridization was performed overnight at 68° C. in ExpressHyb hybridization solution (Clontech Laboratories, Palo Alto Calif., USA,) according to the manufacturer's instructions. The cDNA probe used was labeled with [α]³²P] dCTP using a random primer labeling kit (Megaprime DNA labeling system; Amersham Pharmacia Biotech, Piscataway N.J., USA) and had a specific activity of 1×10⁹ dpm/μg. The blots were washed several times in 2×SSC, 0.05% SDS for 30-40 minutes at room temperature, and were then washed in 0.1×SSC, 0.1% SDS for 40 minutes at 50° C. (see Sambrook et al, 1989, “Molecular Cloning, A Laboratory Manual”, Cold Spring Harbor Press, New York, USA). The blots were covered with standard domestic plastic wrap and exposed to an X-ray film at −70° C. with two intensifying screens for 36 hours.

In AF212372 the mouse Spinl1 mRNA is described as a transcript of 2211 bp in size. The results of this experiment indicate that mouse Spinl1 is expressed as an approximately 2250 bp transcript in several tissues, with a strong 2250 bp signal detected in trachea, lung, esophagus, small intestine, kidney, skeletal muscle, testis, brain (left hemisphere, cerebellum), and spinal cord. Weak signals of 2250 bp were detected in large intestine, white adipose tissue, brown adipose tissue, liver, and bladder (see FIG. 9B). Another Spinl1-specific transcript of approximately 4900 bp in size might indicate an incompletely-spliced pre-mRNA. A strong signal of the 4900 bp band was detected in trachea and thymus. Weak signals of the 4900 bp band were detected in all tissues positive for the 2250 bp band. A single band of approximately 1400 bp, detected in testis only, might represent a tissue-specific splice variant.

### Example 12

### Characterization of Spinl1 Proteins from Different Species—Amino Acid Conservation

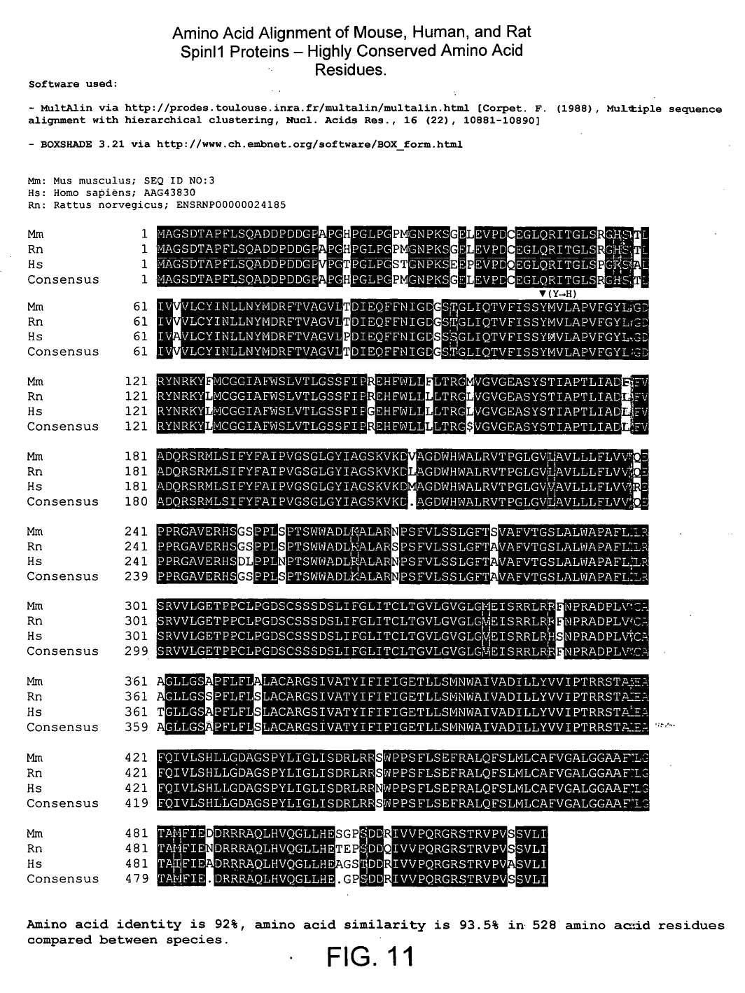

1. In an inter-species comparison of mouse (SEQ ID NO:3), rat (SEQ ID NO:40), and human (SEQ ID NO:7) Spinl1 protein amino acids, the overall degree of identity is 92%, whereas the degree of similarity reaches 93.5%. The high degree of amino acid identity and similarity is indicative for highly conserved residues between the species (see FIG. 11 and Table 1), indicating functional significance of these conserved residues in the peptides compared in this Example. The amino acid that is exchanged in the Spinl1 phenotype, 108Y, is identical between the compared species (see arrow in FIG. 11).

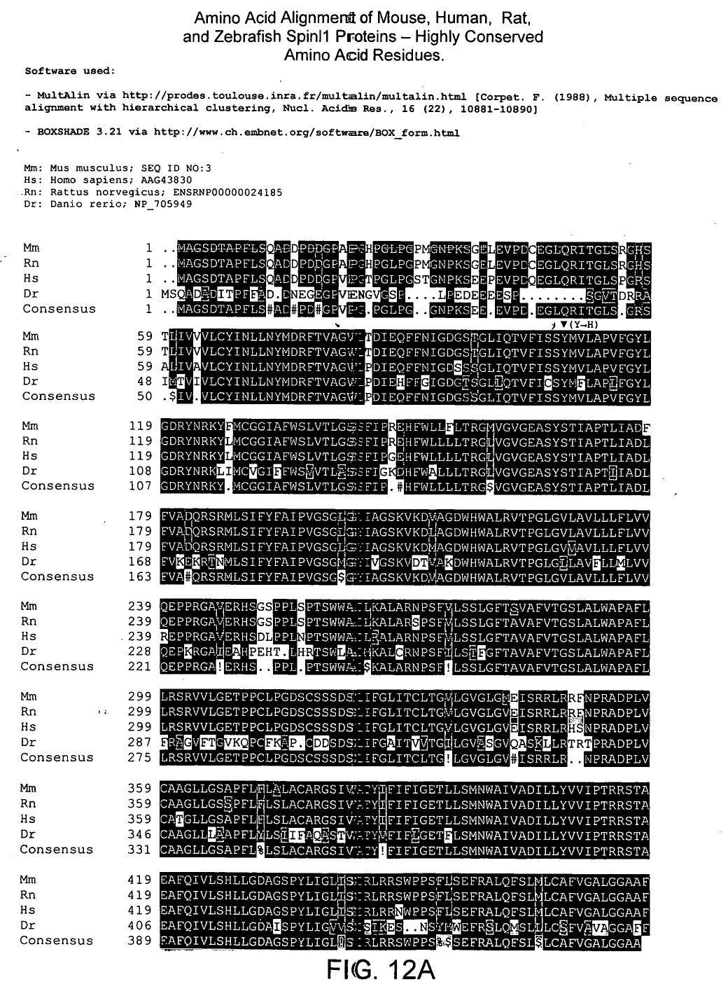

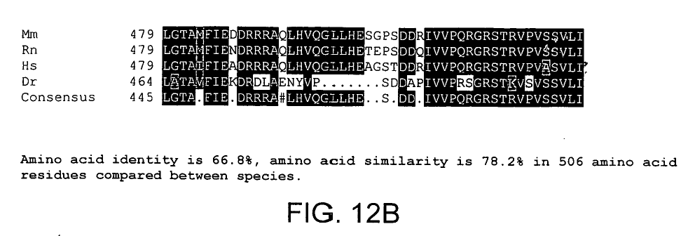

2. In an inter-species comparison of mouse (SEQ ID NO:3), rat (SEQ ID NO:40), human (SEQ ID NO:7) and zebrafish (SEQ ID NO:41) Spinl1 protein amino acids, the overall degree of identity within a 506 amino acid residue stretch is 66.8%, whereas the degree of similarity reaches 78.2%. The high degree of amino acid identity and similarity is indicative for highly conserved residues between the species (see FIG. 12 and Table 2), indicating functional significance of these conserved residues in the peptides compared in this Example. Again, the amino acid that is exchanged in the Spinl1 phenotype, 108Y, is identical between the compared species (see arrow in FIG. 12).

3. In an inter-species comparison of mouse (SEQ ID NO:3), rat (SEQ ID NO:40), human (SEQ ID NO:7), zebrafish (SEQ ID NO:41), and fugu (SEQ ID NO:42) Spinl1 protein amino acids, the overall degree of identity within a 414 amino acid residue stretch is 64%, whereas the degree of similarity reaches 77%. The degree of amino acid identity and similarity is indicative for highly conserved residues between the species (see FIG. 13 and Table 3), indicating functional significance of these conserved residues in the peptides compared. The amino acid exchanged in the Spinl1 phenotype, 108Y, is identical between the species compared in this Example (see arrow in FIG. 13).

Evolutionary pressure has conserved these residues at their particular locations in the molecule. It is predicted that any non-conservative aa substitution will modify the peptide's normal biological function in a manner analogous to that observed in the present invention. Hence, identification of such an abnormal Spinl1 peptide sequence in a biological sample, or of the cDNA encoding such an abnormal Spinl1 peptide, will be indicative of an increased probability of developing the phenotype of the present invention.

### Example 13

### Cloning of Mouse and Human Spinl1 into Expression Vectors

To express the wild type or mutant Spinl1 in bacteria or eukaryotic cells, the cDNA can be cloned into an expression vector using standard cloning and transfection techniques, as described, for instance, in Sambrook et al. (eds.), MOLECULAR CLONING: A LABORATORY MANUAL (2^{nd} Ed.), Cold Spring Harbor Laboratory Press, Cold Spring Harbor, N.Y., 1989; and Ausubel et al. (eds.), CURRENT PROTOCOLS IN MOLECULAR BIOLOGY, John Wiley & Sons, New York, N.Y., 1993. A preferred method is the cDNA subcloning into expression vectors of the Gateway cloning and expression system (Invitrogen, California, USA), according to the manufacturer's instructions.

Purification of recombinant Spinl1 from host cells can be performed using standard methods well-known to those skilled in the art. For standard references, see above.

### Example 14

### Gene Therapy

A number of viruses, including retroviruses, adenoviruses, herpes viruses, and pox viruses, have been developed as live viral vectors for gene therapy. A nucleic acid that codes for mutated human Spinl1 protein (SEQ ID NO:8) or wild type human Spinl1 protein (SEQ ID NO:7) is inserted into the genome of a parent virus to allow them to be expressed by that virus. This is accomplished by first constructing a DNA donor vector for in vivo recombination with a parent virus.

The DNA donor vector contains (i) a prokaryotic origin of replication, so that the vector may be amplified in a prokaryotic host; (ii) a gene encoding a marker which allows selection of prokaryotic host cells that contain the vector (e.g., a gene encoding antibiotic resistance); (iii) at least one gene encoding a desired protein located adjacent to a transcriptional promoter capable of directing the expression of the gene; and (iv) DNA sequences homologous to the region of the parent virus genome where the foreign gene(s) will be inserted, flanking the construct of element (iii).

The donor vector further contains additional genes which encodes one or more marker(s) which will allow identification of recombinant viruses containing inserted foreign DNA. The marker genes to be used include genes that encode antibiotic or chemical resistance (see, e.g., Franke, Rice, Strauss, and Hruby 1985; Falkner and Moss 1988; and Spyropoulos, Roberts, Panicali, and Cohen 1988), as well as genes such as the E. coli lacZ gene that permit identification of recombinant viral plaques by calorimetric assay (Panicali, Grzelecki, and Huang 1986).

Homologous recombination between the donor plasmid DNA and the viral DNA in an infected cell are made using standard techniques. The recombination results in the formation of recombinant viruses that incorporate the nucleic acid encoding SEQ ID NO:8 for mutated Spinl1 or SEQ ID NO:7 for wild type Spinl1. Appropriate host cells for in vivo recombination are eukaryotic cells that can be infected by the virus and transfected by the plasmid vector such as chick embryo fibroblasts, HuTK143 (human) cells, and CV-1 and BSC-40 (both monkey kidney) cells. Infection of cells by the virus and transfection of these cells with plasmid vectors is accomplished by techniques standard in the art.

Following in vivo recombination, recombinant viral progeny are identified by co-integration of a gene encoding a marker or indicator gene with the foreign gene(s) of interest, which, in this case, is the β-galactosidase gene. The presence of the. β-galactosidase gene is selected using the chromogenic substrate 5-bromo-chloro-3-indolyl-β-D-galactosidase (Panicali, Grzelecki, and Huang 1986). Recombinant virus appears as blue plaques in the host cell. Expression of the polypeptide encoded by the inserted gene is further conformed by in situ enzyme immunoassay performed on viral plaques and confirmed by Western blot analysis, radioimmunoprecipitation (RIPA), and enzyme immunoassay (EIA). Positive viruses are cultured, expanded and stored.

### Example 15

### siRNA Generation and Use in Therapy

Production of RNAs

Sense RNA (ssRNA) and antisense RNA (asRNA) of the Spinl1 are produced using known methods such as transcription in RNA expression vectors. In the initial experiments, the sense and antisense RNA are about 500 bases in length each. The produced ssRNA and asRNA (0.5 μm) in 10 mM Tris-HCl (pH 7.5) with 20 mM NaCl are heated to 95° C. for 1 min, then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide (Sambrook et al., Molecular Cloning. Cold Spring Harbor Laboratory Press, Plainview, N.Y. (1989)).

Lysate Preparation

Untreated rabbit reticulocyte lysate (Ambion) is assembled according to the manufacturer's protocoll. DsRNA is incubated in the lysate at 30° C. for 10 min prior to the addition of mRNAs. Then Spinl1 mRNAs are added and the incubation is continued for an additional 60 min. The molar ratio of double stranded RNA and mRNA is about 200:1. The Spinl1 mRNA is radiolabeled (using known techniques) and its stability is monitored by gel electrophoresis.

In a parallel experiment made with the same conditions, the double stranded RNA is internally radiolabeled with α-³²P-ATP. Reactions are stopped by the addition of 2× proteinase K buffer and deproteinized as described previously (Tuschl, Zamore, Lehmann, Bartel, and Sharp 1999b). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bp, is eluted. The efficacy of these 21-23mers for suppressing Spinl1 transcription is assayed in vitro using the same rabbit reticulocyte assay described above using 50 nanomolar of double stranded 21-23 mer for each assay. The sequence of these 21-23 mers is then determined using standard nucleic acid sequencing techniques.

RNA Preparation

21 nt RNAs based on the sequence determined above are chemically synthesized using Expedite RNA phosphoramidites and thymidine phosphoramidite (Proligo, Germany). Synthetic oligonucleotides are deprotected and gel-purified (Elbashir, Lendeckel, and Tuschl 2001b) xx16), followed by Sep-Pak C18 cartridge (Waters, Milford, Mass., USA) purification (Tuschl, Ng, Pieken, Benseler, and Eckstein 1993) xx47).

These RNAs (20 μM) single strands are incubated in annealing buffer (100 mM potassium acetate, 30 mM HEPES-KOH at pH 7.4, 2 mM magnesium acetate) for 1 min at 90° C, followed by 1 h at 37° C.

Cell Culture

Cell cultures that regularly express Spinl1, including, but not limited to 3T3-L1 (pre-adipocyte cell line) are propagated using standard conditions. 24 hours before transfection, at approx. 80% confluency, the cells are trypsinized and diluted 1:5 with fresh medium without antibiotics (1-3×10⁵ cells/ml) and transferred into 24-well plates (500 μl/well). Transfection is performed using a commercially available lypofection kit and Spinl1 expression is monitored using standard techniques with a positive and a negative control. As positive control cells are used that naturally express Spinl1 while as negative control cells are used that do not express Spinl1. It is seen that base-paired 21-23 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

As an alternative approach, double stranded oligonucleotides of 63-67 base pairs in length, representing templates cloned into a vector system pSilencer™ 2.1-U6 neo and targeting the particular murine Spinl1 nucleotide sequences described in SEQ ID NOS:43, 44, 45, 46, 47, or 48, were synthesized and processed. The 63- to 67mer oligonucleotides (see SEQ ID NOS:49, 50, 51, 52, 53, 54, 55, 56, 57, 58, 59, and 60) comprise a first Spinl1 nucleotide to be transcribed (guanidine), a loop sequence of 9 bases, a sequence which is revers complementary to a target sequence, six thymidine residues (serving as transcription stop signal for the RNA Polymerase III), a sequence motif GGAA recommended by AMBION Inc., and sequences for generating the required restriction enzyme cloning sites BamHI and HindIII. Reverse complementary oligonucleotides were annealed: the oligonucleotide of SEQ ID NO:49 to the oligonucleotide of SEQ ID NO:50, the oligonucleotide of SEQ ID NO:51 to the oligonucleotide of SEQ ID NO:52, the oligonucleotide of SEQ ID NO:53 to the oligonucleotide of SEQ ID NO:54, the oligonucleotide of SEQ ID NO:55 to the oligonucleotide of SEQ ID NO:56, the oligonucleotide of SEQ ID NO:57 to the oligonucleotide of SEQ ID NO:58, and the oligonucleotide of SEQ ID NO:59 to the oligonucleotide of SEQ ID NO:60. Double stranded oligonucleotides were subsequently cloned into pSilencer™ 2.1-U6 neo (AMBION Cat# 5764), following the manufacturer's instruction manual for Cat #5764, 5770. Culture cell transfection was performed as described above.

The templates target Spinl1 nucleotide sequences described in SEQ ID NO:43 (5′-AAGCAGATGATCCTGATGACG; coding position +35 to +55), SEQ ID NO:44 (5′-AGGACCCATGGGGAATCCAAA, coding position +84 to +104), SEQ ID NO:45 (5′-GCTACATTAACCTCCTGAACT, coding position +197 to +217), SEQ ID NO:46 (5′-AAGGCACTGGCACGAAATCCT, coding position +793 to +813), SEQ ID NO:47 (5′-AGCATCGTGGCCACCTATAT, coding position +1138 to +1157) or SEQ ID NO:48 (5′-AGCCCTTACCTCATFGGTCTA, coding position +1300 to +1320).

The above methods provide ways both for the deduction of suitable Spinl1 siRNA sequences and the use of such siRNAs for in vitro suppression. In vivo suppression may be performed using the same siRNA using well known in vivo transfection or gene therapy transfection techniques.

### Example 16

### Spinl1 Mutations Resulting in Abnormal Spinl1 Protein Expression Levels

It is predicted that any mutation in the Spinl1 gene resulting in abnormal Spinl1 protein expression levels in an individual will interfere with the protein's normal biological function, including in a manner analogous to that observed in the present invention. Mutations leading to abnormal Spinl1 protein expression levels might affect any aspect of gene expression, e.g., DNA transcription, mRNA transport and processing, mRNA translation or Spinl1 protein transport or half-life itself.

For instance, identification of an abnormal Spinl1 protein level in a biological sample will be indicative of an increased probability of developing the phenotype of the present invention. Methods for quantifying the protein expression levels in a biological sample are well known in the art. Spinl1 protein levels may be analysed, e.g., by obtaining a biopsy from an individual and quantifying the amount of Spinl1 protein by the use of an antibody or any other probe specifically recognizing the Spinl1 protein, e.g., using an ELISA or a Western blot.

Alternatively, identification of an abnormal Spinl1 mRNA level in a biological sample will be indicative of an increased probability of developing the phenotype of the present invention. Methods for quantifying the mRNA expression levels in a biological sample are well known in the art. Spinl1 mRNA levels may be analysed, e.g., by obtaining a biopsy from an individual and quantifying the amount of Spinl1 mRNA by the use of quantitative RT-PCR or any other method relying on probes specifically recognizing the Spinl1 mRNA.

Alternatively, identification of an abnormal Spinl1 mRNA transport and processing in a biological sample will be indicative of an increased probability of developing the phenotype of the present invention. Spinl1 mRNA processing may be analysed, e.g., by obtaining a biopsy from an individual and quantifying the processing of mRNA by the use of Northern blotting or qualitative RT-PCR or any other method relying on probes specifically recognizing the Spinl1 mRNA processing.

Moreover, any given mutation in the Spinl1 gene may be tested for its effect on Spinl1 expression by using an appropriate artificial expression system.

For instance, a cDNA encoding any given mutated Spinl1 protein may be isolated and expressed in any suitable expression system. The amount of expressed Spinl1 peptide or mRNA or the Spinl1 mRNA transport and processing may be analysed by using methods analogous to those mentioned above.

Alternatively, regulatory sequences of the Spinl1 gene may be isolated and analysed in any suitable expression system. Expression levels of an appropriate reporter gene would be indicative for the efficiency of the Spinl1 regulatory sequences to direct gene expression.

Once mutations in the Spinl1 gene resulting in abnormal Spinl1 peptide expression levels in an individual or in a suitable expression system are identified, this knowledge may be used to screen any suitable biological sample for the presence of such a mutation by means well known in the art, including sequencing of the individual's Spinl1 cDNA or genomic DNA. Individuals carrying any of the previously characterized mutations will bear an increased risk of developing the phenotype of the present invention.

### Example 17

### Pre-Adipocyte Differentiation into Adipocytes

3T3-L1 cells are obtained from American Type Culture Collection, ATCC, Manassas, USA. Cells are cultured in growth medium containing 10% iron-enriched fetal bovine serum in Dulbecco's modified Eagle's medium. For standard adipocyte differentiation, 2 days after cells reach confluency (referred to as day 0), cells are exposed to differentiation medium, containing 10% fetal bovine serum, 10 μg/ml of insulin, 1 μM Dexamethasone, and 0.5 μM isobutylmethylxanthine, for 48 h. Cells are then maintained in postdifferentiation medium containing 10% fetal bovine serum, and μg/ml insulin. Adipocytes are sensitive to oil red 0 staining, as described: dishes are washed three times with PBS buffer, fixed by 10% formalin in PBS buffer for 1 hour at room temperature. After fixation, cells are washed once with PBS and stained with a filtered oil red 0 stock solution (0.5 g of oil red 0 (Sigma) in 100 ml of isopropyl alcohol) for 15 minutes at room temperature. Cells are then washed twice with water for 15 minutes each and visualized.

### Example 18

### In Vitro Detection of Pharmacological Interference with Spinl1 Function—Compound Screening

Lysosomes, cytoplasmic subcellular particles containing hydrolytic enzymes, are involved in intracellular digestive processes. Endosomes derive from the cell membrane and can fuse with lysosomes. Lysosomes and endosomes are present in practically all animal cells. Several endosomal and lysosomal marker proteins are known, e.g. LAMP1, a lysosome marker protein. Alternatively, hydrolytic enzymes are detectable by Lysotracker (Molecular Probes, Inc., Eugene, USA), an acidotropic reagent.

In Drosophila, Spinl1 is described as a transporter-like protein that colocalizes to the (late) endosome/lysosome compartment of nerve and muscle cells, and that is necessary for the normal architecture of the endosome/lysosome compartment (Sweeney and Davis 2002; Sweeney and Davis 2002). Loss-of-function of endogenous Spinl results in a dramatic expansion of the endosomal and lysosomal compartment, as described in that reference. This observation is useful in establishing an in vitro-assay for detection of specific interaction between Spinl1 protein and compounds.

For example, in a tissue culture assay, Spinl1 expressing cells, like 3T3-L1 pre-adipocyte cells are grown in Alpha minimum essential medium with 2 mM 1-glutamine and 1 mM sodium pyruvate without ribonucleosides and deoxyribonucleosides, 90%; 10% FBS (American Type Culture Collection, ATCC, Manassas, USA) at 37° C. on poly-lysine cover slips until 60-80% confluency. Cells are treated with chemical compounds for several hours, as described for example in Wess, 1999, Structure-Function Analysis of a G Protein-coupled Receptor, John Wiley & Sons, Inc, Publication; and references cited therein. After treatment, cells are washed and fixed for 20 min in 4% paraformaldehyde in PBS, followed by washing in PBS. Fixed cells are permeabilized by dipping into ice-cold methanol after which they are stained with anti-LAMP1 antibody which is visualized using an 1/50 to 1/300 dilution of goat anti-mouse Cy5-conjugated secondary antibody (Chemicon International, Inc., Temecula, USA). Three-dimensional optical reconstitution is achieved using an inverted microscope outfitted for 3D deconvolution microscopy (i.e. Zeiss 200M, Intelligent Imaging Innovations-31; Zeiss, Göttingen, Germany).

Alternatively, after compound treatment cells are washed with HL3 medium, and subsequently stained in HL3/1 mM Ca²⁺ with 0.2 μM Lysotracker Red (Molecular Probes Inc., Eugene, USA) for 3 to 5 minutes at room temperature, followed by rinsing with PBS and paraformaldehyde fixation, as described before. Imaging is performed using, e.g., a Leica laser scanning confocal microscope (TCS-SP) (Leica, Bensheim, Germany). Lysotracker Red dye is excited using the 568 nm laser line and the emission fluorescence is measured between 580-630 nm. Fluorescence detection may also be performed by FACS analysis.

To test for specific interaction between Spinl1 and a particular compound, cells not-expressing Spinl1 are treated as described above for Spinl1 expressing cells. Cells or cell lines not expressing Spinl1 are derived from a tissue not naturally expressing Spinl1, e.g., pancreatic cells lines, like PLA5 and AsPc1 (American Type Culture Collection, ATCC, Manassas, USA). In case of a Spinl1-specific mechanism the compound will not induce size alteration in endosomellysosome compartment, like observed in untreated controls of the non-expressing cell line.

Both methods described are tools for depicting size alterations in endosome/lysosome compartment.

Another possibility to design an in vitro-assay takes advantage of cell free techniques. A Spinl1 preparation is incubated with the compound to be tested for specific binding under physiological conditions. Distinction between a specific association and a mere random mixing of both substances is achieved by NMR (nuclear magnetic resonance) spectra, as described in Combinatorial Chemistry & High Throughput Screening, 2002, 5(8), the whole volume covering NMR-based screening methods; and Bradley, 2001, Modern Drug Discovery 4(11): 28-34.

### Example 19

### Increased Leptin Sensitivity—Spinl1 Mutant Acts as a Leptin Sensitizer

As described in Example 2, Spinl1 homozygous mice display significantly reduced serum leptin levels, whereas the food intake is only slightly elevated (see also FIG. 16). This suggests an elevated leptin sensitivity in the Spinl1 mutant animals, since a reduced leptin level induces the full anorexigenic effect of the hormone.

1. Epistatic Interaction Between the Leptin Pathway and Spinl1

To investigate the epistatic interaction between the leptin pathway and Spinl1, homozygous Spinl1 mice (chg) were crossed to homozygous ob mice (ob), which carry a null allele for the leptin gene. Ob mice are obese. The reduced body weight observed for homozygous Spinl1 mutant animals (chg) is reversed to an obesitas phenotype in the Spinl1/ob double-homozygous animals (chg/ob), comparable to the obesitas phenotype of the ob/ob mice (ob), as depicted in FIG. 17. Chg/ob animals display an almost identical body composition in respect to lean and fat, and in total body weight in comparison to ob mice.

This epistatic interaction shows that the elevated leptin sensitivity of the Spinl1 mutant animals is reversed by a complete interruption of the leptin signalling pathway by the ob mutation.

2. Leptin Induced Phosphorylation of STAT3 and AKT

To test directly for an interaction between leptin signalling pathways and Spinl1 leptin-induced phosphorylation of STAT3 and AKT was studied in liver and in muscle tissue, respectively, of Stiml1 homozygous mutant animals and of wild type animals. Both kinases are known to be involved in the intracellular signalling cascades of activated leptin receptors.

Murine recombinant leptin (Biovision, Freiburg, Germany)) was administered intraperitoneal at a concentration of 2 μg/g body weight to mice at the age of 16 weeks. Mice were sacrified at the time of administration, 15 min, 30 min, and 60 min after administration, respectively. Liver and muscle tissue (musculus soleus) were removed and protein extraction was performed according to standard methods, as described in Maroni et al. (Maroni, P, Bendinelli, P, Piccoletti, R, Molecular and Cellular Endocrinology (2003), 201:109-121): skeletal muscle from control and leptin-treated animals was homogenized, (1:10, weight/volume) respectively, in lysis buffer containing 50 mM Tris pH 7.4, 150 mM NaCl, 2 mM EDTA, 1% NP-40, 10% glycerol, 100 mM NaF, 1 mM MgCl₂, 1 mM CaCl₂, 2 mM Na₃VO₄, 50 mM beta-glycerophosphate, 10 μg/ml aprotinin, 10 μg/ml leupeptin, and 1 mM PMSF, by an ultra-turrax for 60 seconds. Homogenates were kept on agitation for 1 h at 4° C. and then centrifuged at 12000×g for 30 min. A 2 mg aliquot of supernatant proteins was preclarified, using 5 μl of normal rabbit serum and protein A sepharose for 1 h at 4° C. After centrifugation, 5 μg of anti-OB-Rb antibodies (Alpha Diagnostic International, San Antonio, Tex., USA) were added to the clarified supernatant and the samples were incubated over night in a rotary incubator. Samples were then adsorbed to protein A sepharose and collected by centrifugation. The immunocomplexes were sequentially washed with lysis buffer (twice), with 2 mM Na₃VO₄, 10 mM Tris pH 7.4, 50 mM NaCl, 10 μg/ml aprotinin, 10 μg/ml leupeptin and 50 mM beta-glycerophosphate (twice), resuspended in electrophoresis buffer (Laemmli, UK, Nature (1970) 270:680-685), resolved by SDS-PAGE and transferred to PVDF membranes for immunoblot analysis (Western blotting; see in Ausubel at al., (eds.), Current Protocols in Molecular Biology, John Wiley&Sons, New York, 1993). Subsequent detection of phosphorylated and unphosphorylated STAT3 and AKT was performed.

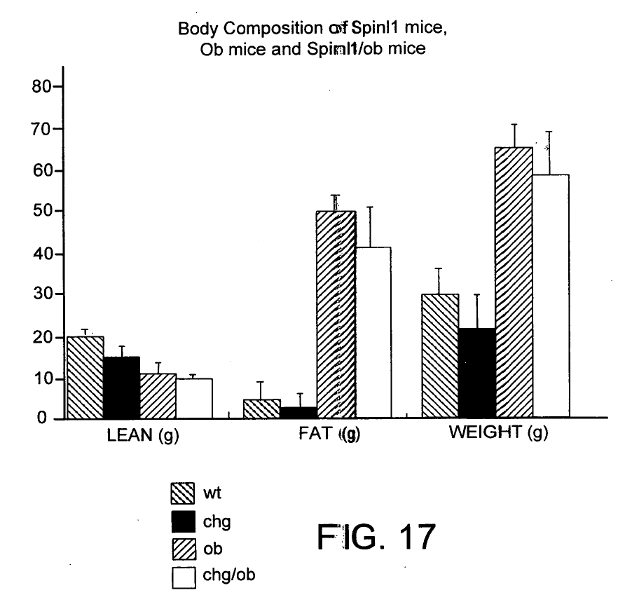

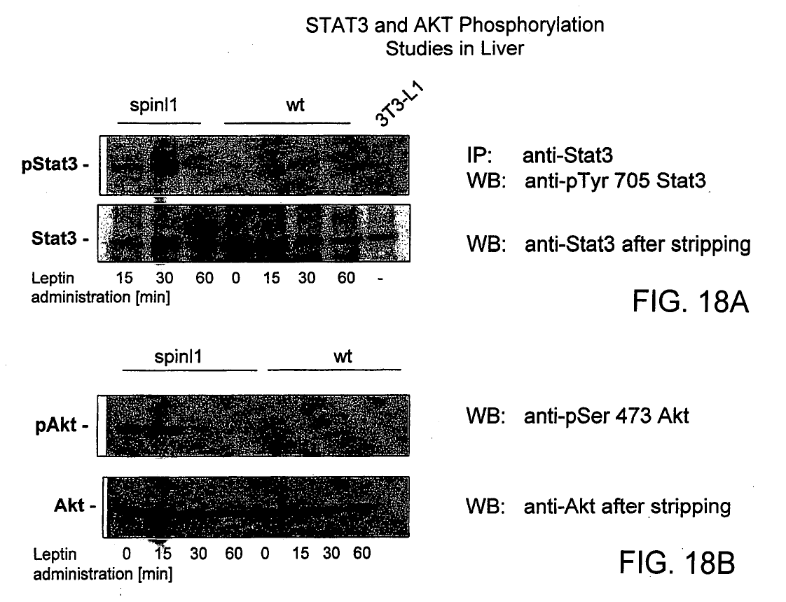

FIG. 18 depicts Western blotting (WB) data, detecting phosphorylated STAT3 (pStat3) and unphosphorylated STAT3 (Stat3) in liver of homozygous Spinl1 mice after administration of leptin for 15, 30, and 60 minutes, respectively. In a control experiment with wild type mice, detection of phosphorylated and unphosphorylated STAT3 was performed at the time of leptin administration start (0) and after administration of leptin for 15, 30, and 60 minutes, respectively. STAT3 was detected by immunoprecipitation (IP) with an anti-STAT3 antibody (anti-Stat3; Upstate Biotechnology, Lake Placid, N.Y.). Selective detection of phosphorylated STAT3 was performed by applying an antibody generated against phosphorylated Tyrosin 705 of STAT3 (WB: anti-pTyr705 Stat3; Cell Signalling). Overall amounts of immunoprecipitated STAT3 was determined by stripping the protein membrane and applying anti-Stat3 (WB: anti-Stat3 after stripping). Phosphorylated STAT3 is undetected in 3T3-L1 cells (3T3-L1), as seen in FIG. 18A. FIG. 18B depicts Western blotting (WB) data, detecting phosphorylated AKT (pAkt) and unphosphorylated AKT (Akt) in liver of homozygous Spinl1 mice after administration of leptin for 15, 30, and 60 minutes, respectively. The state of AKT phosphorylation was also determined at the time of leptin administration start (0). In a control experiment with wild type mice, detection of phosphorylated and unphosphorylated AKT was performed at the time of leptin administration start (0) and after administration of leptin for 15, 30, and 60 minutes, respectively. AKT was detected by immunoprecipitation (IP) with an anti-AKT antibody (anti-Akt). Selective detection of phosphorylated AKT was performed by applying an antibody generated against phosphorylated Serin 473 of AKT (WB: anti-pSer473 Akt; Cell Signalling). Overall amounts of immunoprecipitated AKT was determined by stripping the protein membrane and applying anti-Akt (WB: anti-Akt after stripping).

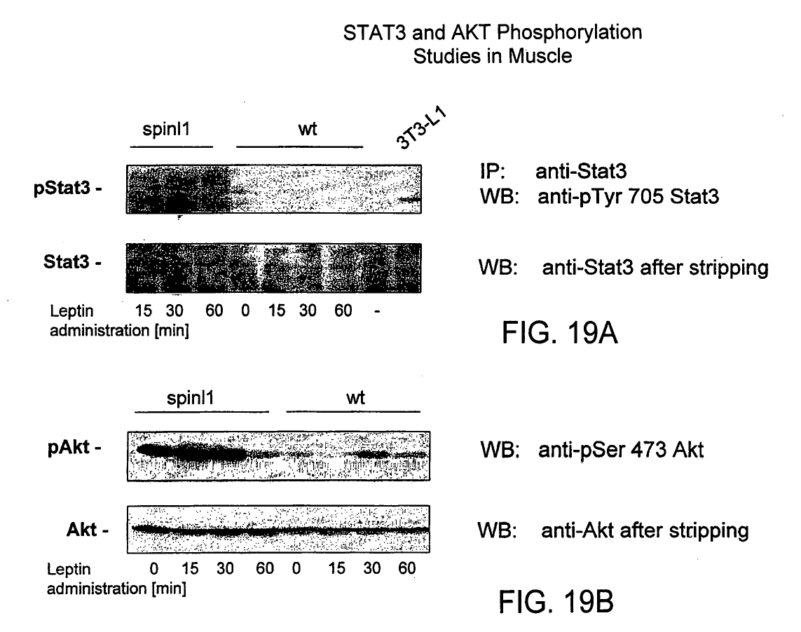

FIG. 19 depicts Western blotting (WB) data, detecting phosphorylated STAT3 (pStat3) and unphosphorylated STAT3 (Stat3) in muscle of homozygous Spinl1 mice after administration of leptin for 15, 30, and 60 minutes, respectively. In a control experiment with wild type mice, detection of phosphorylated and unphosphorylated STAT3 was performed at the time of leptin administration start (0) and after administration of leptin for 15, 30, and 60 minutes, respectively. STAT3 was detected by immunoprecipitation (IP) with an anti-STAT3 antibody (anti-Stat3). Selective detection of phosphorylated STAT3 was performed by applying an antibody generated against phosphorylated Tyrosin 705 of STAT3 (WB: anti-pTyr705 Stat3). Overall amounts of immunoprecipitated STAT3 was determined by stripping the protein membrane and applying anti-Stat3 (WB: anti-Stat3 after stripping). Phosphorylated STAT3 is detected in 3T3-L1 cells (3T3-L1) at low level, as seen in FIG. 19A. FIG. 19B depicts Western blotting (WB) data, detecting phosphorylated AKT (pAkt) and unphosphorylated AKT (Akt) in muscle of homozygous Spinl1 mice after administration of leptin for 15, 30, and 60 minutes, respectively. The state of AKT phosphorylation was also determined at the time of leptin administration start (0). In a control experiment with wild type mice, detection of phosphorylated and unphosphorylated AKT was performed at the time of leptin administration start (0) and after administration of leptin for 15, 30, and 60 minutes, respectively. AKT was detected by immunoprecipitation (IP) with an anti-AKT antibody (anti-Akt). Selective detection of phosphorylated AKT was performed by applying an antibody generated against phosphorylated Serin 473 of AKT (WB: anti-pSer473 Akt). Overall amounts of immunoprecipitaed AKT was determined by stripping the protein membrane and applying anti-Akt (WB: anti-Akt after stripping).

As a result of the experiment phosphorylation of both proteins, STAT3 and AKT was elevated in the homozygous Spinl1 mice compared to wild type controls. As both proteins are known to be involved in the intracellular signalling cascades of activated leptin receptors these results further corroborate the increased leptin sensitivity in the Spinl1 affected mice.

### Example 20

### In Vitro Detection of Pharmacological Interference with Spinl1 Function—Compound Screening With Differentiated Spinl1 Myoblasts

1. Differentiation of Spinl1 myoblasts to myocytes Hind limb muscle of an adult homozygous Spinl1 mouse is removed, rinsed with 70% EtOH, and stored in a cell culture dish in sterile PBS. After washing and removing of PBS, muscle tissue is minced to a slurry with a scalpel under 2 ml/g tissue weight of a mixture of collagenase/dispase/CaCl₂ [1.5 ml of 1.5 U/ml collagenase D (Roche), 1.5 ml of 2.4 U/ml dispase II (Roche), 7.5 μl of 1 M CaCl₂ (final con. 2.5 mM)] for several minutes. Minced tissue is transferred to a 15 ml centrifugation tube, incubated at 37° C. for 45 min, and triturated with a sterile plastic pipette. After filtration through a 100μ cell strainer, the cellular material is centrifuged for 5 min at 150 rpm. The cell pellet is resuspended in 10 ml of medium 1 [400 ml Hams F-10 nutrient mix (Gibco); 100 ml FCS (20% final conc.); 50 μl of 25 μg/m IbFGF (Promega) in sterile 0.5 BSA/PBS, pH 7.4; 5 ml Pen/Strep] and plated on a collagen-coated cell culture dish. Incubation is performed at 37° C./5% CO₂ with medium 1 change every second day of incubation. At day 5 of incubation cells are splitted by aspirating off the medium, and replating myoblasts on a new collagen-coated dish under medium 1. Splitting is repeated until fibroblasts are no longer visible in culture and medium 1 is changed to medium 2 [200 ml Hams F-10 nutrient mix (Gibco), 200 ml DMEM, 100 ml FCS (20% final conc., 50 μl of 25 μg/m IbFGF (Promega) in sterile 0.5 BSA/PBS, pH 7.4; 5 ml Pen/Strep]. Medium 2 is changed every second day. At this stage primary myoblasts can be frozen for storage using standard cell culture protocols. For differentiation of myoblasts to myocytes medium 2 is replaced by fusion medium [95 ml DMEM, 5 ml horse serum (Gibco) (5% final conc.), 1 ml Penicillin/Streptomycin (1% final conc.), ad 100 ml]. Fusion medium is changed daily and cells are splitted at 50% confluency as described before. Within several days, large multinucleated myotubes become visible.

2. Compound-Dependent Alteration of Leptin-Induced Phosphorylation of STAT3 and AKT

For screening of compounds with leptin sensitizer properties terminal differentiated Spinl1 myocyte cells are grown in fusion medium in cell culture dishes until 90% confluency. Cells are incubated with murine recombinant leptin (Biovision, Freiburg, Germany) for 0, 15, 30, and 60 min, respectively. Final concentrations of leptin range between 10 nM and 100 μM. Control incubations are performed without leptin administration. Cells are lysed according to standard methods known in the art and protein extraction is performed according to standard methods, as described in Ausubel et al. (eds.), CURRENT PROTOCOLS IN MOLECULAR BIOLOGY, John Wiley & Sons, New York, N.Y., 1993.

Protein extracts are subjected to Western blotting procedure and subsequent detection of phosphorylated STAT3 and AKT, analogous to the method described in Example 19.2. In case of leptin administration to Spinl1 myocytes, levels of phosphorylated marker proteins STAT3 and AKT will be increased compared to the control without leptin administration. In case of leptin administration to Spinl1 myocytes, followed or accompanied by administration of a compound that has leptin sensitivity promoting function (i.e., that acts as a leptin sensitizer), the levels of phosphorylated marker proteins STAT3 and AKT will be further increased, and/or the kinetics of phosphorylation affected in such a way that phosphorylation occurs more rapidly compared to the incubation of the Spinl1 myocytes with leptin alone. Conversely, in case of leptin administration to Spinl1 myocytes, followed or accompanied by administration of a compound that has leptin sensitivity decreasing function (i.e., that acts as a leptin desensitizer), the levels of phosphorylated marker proteins STAT3 and AKT will be decreased, and/or the kinetics of phosphorylation affected in such a way that phosphorylation occurs less rapidly compared to the incubation of the Spinl1 myocytes with leptin alone. Compounds identified by their phosphorylation-increasing activity to STAT3 and AKT as leptin sensitizers may be subject to further analysis, as described in Example 21.

### Example 21

### In Vitro Detection of Compounds with Leptin Pathway Sensitizing Activity

Candidate compounds, for example compounds detected by a method as described in Example 20.2, or other compounds suspected to have leptin activity promoting function, may be subjected to a cell-based assay reporting leptin sensitizer activity. In this assay, cells that express the leptin receptor are used. Suitable cells are, e.g., hepatocytes or myocytes.

Suitable cells include myocyte cells derived from db/db mice (Coleman, DL, Diabetologica (1973) 9(4):294-298), which are prepared according to Example 20.1. Db/db mice lack the endogenous leptin receptor gene, and thus, may be used as further negative control to show that cultivation of these cells in the presence of leptin does not result in any detectable STAT and/or AKT phosphorylation. The cells are transfected according to methods well known in the art with an expression vector expressing a leptin receptor protein capable of inducing STAT3 and/or AKT phosphorylation, suitably a leptin receptor fusion wherein the leptin receptor part is fused to a detectable marker polypeptide. Proper expression of functional leptin receptor is then monitored by detecting the marker polypeptide. In order to confirm functional leptin receptor expression, the transfected cells are incubated with recombinant leptin (Biovision, Freiburg, Germany) at concentrations, which allow a detectable increase of STAT3 and AKT phosphorylation, compared to non-transfected cells treated under identical conditions. Suitable final leptin concentrations are ranging from 10 nM and 100 μM.

In the assay for detecting compounds with leptin pathway sensitizing activity, cells of the kind described above (i.e., hepatocytes or myocytes, particularly the leptin receptor transfected myocytes derived from db/db mice) are either incubated with no leptin or a subeffective concentration of recombinant leptin, which is not by itself sufficient to result in an increase in STAT3 and/or AKT phosphorylation. Suitable subeffective leptin concentrations are selected from a range between 1 nM and 10 μM. In parallel, the same cells are incubated with no leptin plus a candidate compound or with a subeffective concentration of leptin plus a candidate compound, followed by assaying the phosphorylation status of STAT3 and/or AKT, e.g., in the way as described in Examples 19.2 and 20.2.

An increase in STAT3 and/or AKT phosphorylation after leptin and candidate compound administration compared to the controls will be indicative of a leptin sensitizer function of the candidate compound analyzed.

### Example 22

### Detection of Mutant Spinl1 Proteins with Either Gain-of-Function or Loss-of-Function Activity

1. Production of Mutant Spinl1 Proteins

A PCR product comprising the cDNA sequence of wild type murine Spinl1 or wild type human Spinl1 is generated by PCR using BioTherm-DNA-polymerase (GeneCraft, Germany) according to the manufacturer's protocol. After subsequent subcloning of the PCR fragment into a plasmid vector, e.g., pCR 2.1-TOPO (Invitrogen, Carlsbad, Calif., USA), according to the manufacurer's instructions, plasmid DNA, bearing the correct Spinl1 insert, is subjected to site-directed mutagenesis, using a QuickChange Site-Directed Mutagenesis Kit (Stratagene, La Jolla, Calif., USA), as outlined in the manufacurer's instructions. In brief, the plasmid vector (parental DNA template) and two oligonucleotide primers, each primer complementary to opposite strands of the vector insert and containing a desired point mutation, are denatured and subjected to PCR amplification with a proof-reading DNA polymerase (Pfu Turbo), provided in the kit. Using the non-strand displacing action of Pfu Turbo DNA polymerase, mutagenic primers are incorporated and extended, resulting in nicked circular DNA strands. In a restriction digest with DpnI, only the methylated parental DNA template is susceptible to DpnI digestion. After transformation in XL1-Blue supercompetent cells, provided with the kit, nicks in the mutated (point mutation) plasmid DNA are repaired. Mutation positive colonies are selected and plasmid DNA is isolated, according to the manufacurer's instructions (Stratagene, La Jolla, Calif., USA). cDNAs can be generated with mutations resulting into an amino acid exchange at any position in the protein.

A mutant cDNA is released from the vector by restriction with an appropiate restriction enzyme, followed by subcloning into an expression vector, according to methods well known in the art.

2. Determination of Function of Mutant Spinl1 Proteins

Spinl1 knock-out cells from a Spinl1 knock-out mouse (see Example 9) are generated from muscle tissue by the method as described in Example 20.1. Spinl1 knock-out myocyte cells, which exhibit endogenous leptin receptor activity, are grown in fusion medium in cell culture dishes until 90% confluency. Cells are transfected with a mutant Spinl1 fusion protein according to methods well known in the art In the assay, control cells are transfected with a vector expressing wild type Spinl1 fusion protein. After administration of recombinant leptin (Biovision, Freiburg, Germany) STAT3 and AKT phosphorylation is monitored, as described in Example 19. In parallel, cells transfected with a vector expressing mutant Spinl1 fusion are monitored for STAT3 and AKT phosphorylation.

An increase of STAT3 and AKT phosphorylation, compared to the control transfection, is indicative of a loss-of-function of the corresponding mutation.

A decrease of STAT3 and AKT phosphorylation, compared to the control transfection, is indicative of a gain-of-function of the corresponding mutation.

### Example 23

### Detection of Human Serum Leptin Level

Human serum leptin level is determined by using a commercially available human leptin ELISA kit (Linco Research, SL Charles, Missouri, USA), following the manufacturer's instructions. In brief, whole blood of a human subject (unknown sample) is collected and directly drawn into a provided Vacutainer serum tube free of anticoagulants. Blood is sitting for 30 min at room temperatur for clotting, followed by centrifugation at 2500 rpm for 15 min at 4° C. and subsequent transfer into microtiter plates. The assay is a direct sandwich ELISA based on 1) capture of human leptin molecules from serum samples to the wells of a microtiter plate coated by pre-titered amount of polyclonal rabbit anti-human leptin antibodies; 2) wash away of unbound materials from samples; 3) binding of a biotinylated monoclonal antibody to the captured human leptin; 4) conjugation of alkaline phosphatase to biotinylated antibodies; 5) wash away of free antibody-enzyme conjugates; and 6) quantification of immobilized antibody-enzyme conjugates by monitoring alkaline phosphatase avtivities in the presence of the substrate p-nitrophenyl phosphate. The enzyme activity is measured spectrophotometrically by the increased absorbancy at 405 nm due to the production of the yellow colored product p-nitrophenol. Since the increase in absorbancy is directly proportional to the amount of captured human leptin in the unknown sample, the latter can be derived by interpolation from a reference curve generated in the same assay with reference standards of known concentrations of human leptin.

### Example 24

### Insulin Resistance Test

Five of each Spinl1 affected mice and wild type mice at the age of 22 weeks were starved over night. Blood was taken from the tail vein cut with a pair of scissors, with 5 μl each collected in a capillary (end to end capillary, Kabe Labortechnik GmbH; Germany) at nine different time points: before insulin injection, and at eight time points after insulin injection. Insulin (Insulin Actrapid HM40 I.E/ml (ge), human; NovoNordisk Pharma GmbH, Germany; 0.7 I.U./kg body weight in 0.9% NaCl) injection was done intraperitoneally. Blood probes were subject to Hitachi analysis (Hitachi 912; Roche Diagnostic, Mannheim, Germany) according to the manufacturer's instructions. Glucose levels of Spinl1 affected and of wild type mice at different time points are indicated as percentage relative to the glucose level determined before insulin injection (see FIG. 21).

According to the Insulin Resistance Test the Spinl1 affected mice showed an increased sensitivity towards Insulin compared to wild type mice, thus indicating that the Spinl1 mutant acts as an Insulin sensitizer.

This invention has been described in detail including the preferred embodiments thereof. However, it will be appreciated that those skilled in the art, upon consideration of this disclosure, may make modifications and improvements thereon without departing from the spirit and scope of the invention as set forth in the claims. All references, patents, patent applications and Genbank references recited in this patent application are hereby incorporated by reference in their entirety.

### REFERENCES

1. Abu-Elheiga, L., M. M. Matzuk, K. A. Abo-Hashema, and S. J. Wakil. 2001. Continuous fatty acid oxidation and reduced fat storage in mice lacking acetyl-CoA carboxylase 2. Science 291: 2613-2616.
2. Barouch, L. A., Berkowitz, D. E., Harrison, R. W., O'Donnell, C. P., and Hare, J. M. 2003. Disruption of leptin signalling contributes to cardiac hypertrophy independently of body weight in mice. Circulation 108(6): 754-759.
3. Bernstein, C. N., and Leslie, W. D. 2003. The pathophysiology of bone disease in gastrointestinal disease. Eur. J. Gastroentreol. Hepatol. 15(8): 857-864.
4. Campochiaro, P. A. 2000. Retinal and choroidal neovascularization. J. Cell Physiol 184: 301-310.
5. Carmeliet, P. 2000. Mechanisms of angiogenesis and arteriogenesis. Nat Med 6: 389-395.
6. Carmeliet, P. and R. K. Jain. 2000. Angiogenesis in cancer and other diseases. Nature 407: 249-257.
7. Coen, G., Ballanti, P., Fischer, M. S., Balducci, A., Calbria, S., Colamarco, L., DiZazzo, G., Lifrieri, F., Manni, M., Sardella, D., Nofroni, I., Bonucci, E. 2003. Serum leptin in dialysis renal osteodystrophy. Am. J. Kidney Dis. 42(5): 1036-1042.
8. Cohen, P., M. Miyazaki, N. D. Socci, A. Hagge-Greenberg, W. Liedtke, A. A. Soukas, R. Sharma, L. C. Hudgins, J. M. Ntambi, and J. M. Friedman. 2002. Role for stearoyl-CoA desaturase-1 in leptin-mediated weight loss. Science 297: 240-243.
9. Corpet, F. 1988. Multiple sequence alignment with hierarchical clustering. Nucleic Acids Res 16: 10881-10890.
10. Dunn-Meynell, A. A, V. H. Routh, L. Kang, L. Gaspers, and B. E. Levin. 2002. Glucokinase is the likely mediator of glucosensing in both glucose-excited and glucose-inhibited central neurons. Diabetes 51: 2056-2065.
11. Elbashir, S. M., W. Lendeckel, and T. Tuschl. 2001b. RNA interference is mediated by 21- and 22-nucleotide RNAs. Genes Dev. 15: 188-200.
12. Elbashir, S. M., W. Lendeckel, and T. Tuschl. 2001a RNA interference is mediated by 21- and 22-nucleotide RNAs. Genes Dev. 15: 188-200.
13. Elbashir, S. M., J. Martinez, A. Patkaniowska, W. Lendeckel, and T. Tuschl. 2001a Functional anatomy of siRNAs for mediating efficient RNAi in Drosophila melanogaster embryo lysate. EMBO J. 20: 6877-6888.
14. Elbashir, S. M., J. Martinez, A. Patkaniowska, W. Lendeckel, and T. Tuschl. 2001b. Functional anatomy of siRNAs for mediating efficient RNAi in Drosophila melanogaster embryo lysate. EMBO J. 20: 6877-6888.
15. Evereklioglu, C., Doganay, S., Er, H., Cekmen, M., and Otlu, B. 2003. Serum leptin cooncentrations are decreased and correlated with disease severity in age-related macular degeneration: a preliminary study. Eye 17(3): 350-355
16. Falkner, F. G. and B. Moss. 1988. Escherichia coli gpt gene provides dominant selection for vaccinia virus open reading frame expression vectors. J. Virol 62: 1849-1854.
17. Franke, C. A., C. M. Rice, J. H. Strauss, and D. E. Hruby. 1985. Neomycin resistance as a dominant selectable marker for selection and isolation of vaccinia virus recombinants. Mol Cell Biol 5: 1918-1924.
18. Furuta, H., M. Furuta, T. Sanke, K. Ekawa, T. Hanabusa, M. Nishi, H. Sasaki, and K. Nanjo. 2002. Nonsense and missense mutations in the human hepatocyte nuclear factor-1 beta gene (TCF2) and their relation to type 2 diabetes in Japanese. J. Clin. Endocrinol. Metab 87: 3859-3863.
19. Hani, E. H., L. Suaud, P. Boutin, J. C. Chevre, E. Durand, A. Philippi, F. Demenais, N. Vionnet, H. Furuta, G. Velho, G. I. Bell, B. Laine, and P. Froguel. 1998. A missense mutation in hepatocyte nuclear factor4 alpha, resulting in a reduced transactivation activity, in human late-onset non-insulin-dependent diabetes mellitus. J. Clin. Invest 101: 521-526.
20. Harborth, J., S. M. Elbashir, K. Bechert, T. Tuschl, and K. Weber. 2001. Identification of essential genes in cultured mammalian cells using small interfering RNAs. J. Cell Sci. 114: 4557-4565.
21. Hegele, R. A., H. Cao, S. B. Harris, A. J. Hanley, and B. Zinman. 1999. The hepatic nuclear factor-1alpha G319S variant is associated with early-onset type 2 diabetes in Canadian Oji-Cree. J. Clin. Endocrinol. Metab 84: 1077-1082.
22. Higgins, D. G. and P. M. Sharp. 1988. CLUSTAL: a package for performing multiple sequence alignment on a microcomputer. Gene 73: 237-244.
23. Holash, J., S. J. Wiegand, and G. D. Yancopoulos. 1999. New model of tumor angiogenesis: dynamic balance between vessel regression and growth mediated by angiopoietins and VEGF. Oncogene 18: 5356-5362.
24. Holtkamp, K., Herpertz-Dahlmann, B., Mika, C., Heer, M., Neussen, M., Fichter, M., Herpertz, S., Senf, W., Blum, W. F., Schweiger, U., Wamke, A., Ballauff, A., Remschmidt, H., and Hebebrand, J. 2003. Elevated physical activity and low leptin levels co-occur in patients with anorexia nervosa. J. Clin Endocrinol. Metab. 88(11): 5169-5174.
25. Hopp, T. P. and K. R. Woods. 1981. Prediction of protein antigenic determinants from amino acid sequences. Proc. Natl. Acad. Sci. U.S.A 78: 3824-3828.
26. Kerbel, R. S. 2000. Tumor angiogenesis: past, present and the near future. Carcinogenesis 21: 505-515.
27. Kohler, G. and C. Milstein. 1975. Continuous cultures of fused cells secreting antibody of predefined specificity. Nature 256: 495-497.
28. Kozbor, D., P. Tripputi, J. C. Roder, and C. M. Croce. 1984. A human hybrid myeloma for production of human monoclonal antibodies. J. Immunol. 133: 3001-3005.
29. Kusari, J., U.S. Verma, J. B. Buse, R. R. Henry, and J. M. Olefsky. 1991. Analysis of the gene sequences of the insulin receptor and the insulin-sensitive glucose transporter (GLUT-4) in patients with common-type non-insulin-dependent diabetes mellitus. J. Clin. Invest 88: 1323-1330.
30. Kyte, J. and R. F. Doolittle. 1982. A simple method for displaying the hydropathic character of a protein. J. Mol. Biol 157: 105-132.
31. Morrison, S. L. 1994. Immunology. Success in specification. Nature 368: 812-813.
32. Munson, P. J. and D. Rodbard. 1980. Ligand: a versatile computerized approach for characterization of ligand-binding systems. Anal. Biochem. 107: 220-239.
33. Nakano, Y., K. Fujitani, J. Kurihara, J. Ragan, K Usui-Aoki, L. Shimoda, T. Lukacsovich, K. Suzuki, M. Sezaki, Y. Sano, R Ueda, W. Awano, M. Kaneda, M. Umeda, and D. Yamamoto. 2001. Mutations in the novel membrane protein spinster interfere with programmed cell death and cause neural degeneration in Drosophila melanogaster. Mol. Cell Biol 21: 3775-3788.
34. Novials, A., J. Vidal, C. Franco, F. Ribera, A. Sener, W. J. Malaisse, and R Gomis. 1997. Mutation in the calcium-binding domain of the mitochondrial glycerophosphate dehydrogenase gene in a family of diabetic subjects. Biochem. Biophys. Res Commun. 231: 570-572.
35. Ozturk, L., Unal, M., Tamer, L., and Celikoglu, F. 2003. The association of the severity of obstructive sleep apnea with plasma leptin levels. Arch Otolaryngol Head Neck Surg. 129(5): 538-540.
36. Panicali, D., A. Grzelecki, and C. Huang. 1986. Vaccinia virus vectors utilizing the beta-galactosidase assay for rapid selection of recombinant viruses and measurement of gene expression. Gene 47: 193-199.
37. Prchal, J. F. and J. T. Prchal. 1999. Molecular basis for polycythemia Curr Opin Hematol. 6:100-109.
38. Reischl, I. G., G. R. Dubois, S. Peiritsch, K-S. Brown, L. Wheat, M. Woisetschlager, and G. C. Mudde. 2000. Regulation of Fc epsilonRI expression on human monocytic cells by ligand and IL-4. Clin. Exp. Allergy 30: 1033-1040.
39. Saglam, K., Aydur, E., Yilmaz, M., and Goktasa, S. 2003. Leptin influences cellular differentiation and progression in prostate cancer. 2003. J. Urol. 169(4): 1308-1311.
40. Schulz, S., Birkenmeier, G., Schagdarsurebgin, U., Wenzel, K., Mueller-Werdan, U., Rehfeld, D., Suss, T., Kabisch, A., Werdan, K., and Glaser, C. 2003. Role of LDL receptor-related protein (LRP) in coronary atherosclerosis. Int. J. Cardiol. 92(2-3): 137-144.
41. Shimokawa, T., M. V. Kumar, and M. D. Lane. 2002. Effect of a fatty acid synthase inhibitor on food intake and expression of hypothalamic neuropeptides. Proc. Natl. Acad. Sci. U.S.A 99: 66-71.
42. Song, Z., B. E. Levin, J. J. McArdle, N. Bakhos, and V. H. Routh. 2001. Convergence of pre- and postsynaptic influences on glucosensing neurons in the ventromedial hypothalamic nucleus. Diabetes 50: 2673-2681.
43. Spyropoulos, D. D., B. E. Roberts, D. L. Panicali, and L. K. Cohen. 1988. Delineation of the viral products of recombination in vaccinia virus-infected cells. J. Virol 62: 1046-1054.
44. Stenwinkel, P., Pecoits-Filho, R., and Lindholm, B. 2003. Leptin, ghrelin, and proinflammatory cytokines: compounds with nutritional impact in chronic kidney disease? Adv. Ren. Replace. Ther. 10(4): 332-345.
45. Sweeney, S. T. and G. W. Davis. 2002. Unrestricted Synaptic Growth in spinster-a Late Endosomal Protein Implicated in TGF-beta-Mediated Synaptic Growth Regulation. Neuron 36: 403-416.
46. Tanizawa, Y., A. C. Riggs, KC. Chiu, R. C. Janssen, D. S. Bell, R. P. Go, J. M. Roseman, R. T. Acton, and M. A. Permutt. 1994. Variability of the pancreatic islet beta cell/liver (GLUT 2) glucose transporter gene in NIDDM patients. Diabetologia 37: 420-427.
47. Tatusova, T. A. and T. L. Madden. 1999. BLAST 2 Sequences, a new tool for comparing protein and nucleotide sequences. FEMS Microbiol. Lett 174: 247-250.
48. Tuschl, T., M. M. Ng, W. Pieken, F. Benseler, and F. Eckstein. 1993. Importance of exocyclic base functional groups of central core guanosines for hammerhead ribozyme activity. Biochemistry 32: 11658-11668.
49. Tuschl, T., P. D. Zamore, R Lehmann, D. P. Bartel, and P. A. Sharp. 1999b. Targeted mRNA degradation by double-stranded RNA in vitro. Genes Dev. 13: 3191-3197.
50. Tuschl, T., P. D. Zamore, R. Lehmann, D. P. Bartel, and P. A. Sharp. 1999a Targeted mRNA degradation by double-stranded RNA in vitro. Genes Dev. 13: 3191-3197.
51. Usui-Aoki, K, Y. Nakano, and D. Yamamoto. 2002. Pathology of the adult central nervous system induced by genetic inhibition of programmed cell death in Drosophila pupae. Arch. Insect Biochem. Physiol 49: 94-101.
52. Vinores, S. A., N. L. Derevjanik, M. A. Vinores, N. Okamoto, and P. A. Campochiaro. 2000. Sensitivity of different vascular beds in the eye to neovascularization and blood-retinal barrier breakdown in VEGF transgenic mice. Adv. Exp. Med. Biol 476: 129-138.
53. Vitetta, E. S., K. A. Krolick, M. Miyama-Inaba, W. Cushley, and J. W. Uhr. 1983. Immunotoxins: a new approach to cancer therapy. Science 219: 644-650.
54. Waeber, G., J. Delplanque, C. Bonny, V. Mooser, M. Steinmann, C. Widmann, A. Maillard, J. Miklossy, C. Dina, E. H. Hani, N. Vionnet, P. Nicod, P. Boutin, and P. Froguel. 2000. The gene MAPK81P1, encoding islet-brain-1, is a candidate for type 2 diabetes. Nat Genet 24: 291-295.
55. Yamamoto, D., J. M. Jallon, and A. Komatsu. 1997. Genetic dissection of sexual behavior in Drosophila melanogaster. Annu. Rev. Entomol. 42:551-85: 551-585.
56. Young, R. M., S. Marty, Y. Nakano, H. Wang, D. Yamamoto, S. Lin, and M. L. Allende. 2002. Zebrafish yolk-specific not really started (nrs) gene is a vertebrate homolog of the Drosophila spinster gene and is essential for embryogenesis. Dev. Dyn. 223: 298-305.
57. Yuan, F., Chen, Y., Dellian, M., Safabakhsh, N., Ferrara, N., and Jain, R. K. 1996. Time-dependent vascular regression and permeability changes in established human tumor xenografts induced by an anti-vascular endothelial growth factor/vascular permeability factor antibody. Proc Natl Acad Sci USA 93: 14765-14770.
58. Verhoeyen, M., Milstein, C., and Winter, G. (1988a). Reshaping human antibodies: grafting an antilysozyme activity. Science 239: 1534-1536.
59. Riechmann, L., Clark, M., Waldmann, H., and Winter, G. (1988b). Reshaping human antibodies for therapy. Nature 332: 323-327.
60. Jones, P. T., Dear, P. H., Foote, J., Neuberger, M. S., and Winter, G. (1986). Replacing the complementarity-determining regions in a human antibody with those from a mouse. Nature 321: 522-525.
61. Cote, R. J., Morrissey, D. M., Houghton, A. N., Beattie, E. J., Jr., Oettgen, H. F., and Old, L. J. (1983). Generation of human monoclonal antibodies reactive with cellular antigens. Proc. Natl. Acad. Sci. U.S. A 80: 2026-2030.
62. Marks, J. D., Griffiths, A. D., Malnqvist, M., Clackson, T. P., Bye, J. M., and Winter, G. (1992). By-passing immunization: building high affinity human antibodies by chain shuffling. Biotechnology (N.Y.) 10: 779-783.
63. Marks, J. D., Hoogenboom, H. R., Bonnert, T. P., McCafferty, J., Griffiths, A. D., and Winter, G. (1991a). By-passing immunization. Human antibodies from V-gene libraries displayed on phage. J. Mol. Biol 222: 581-597.
64. Marks, J. D., Hoogenboom, H. R., Bonnert, T. P., McCafferty, J., Griffiths, A. D., and Winter, G. (1991b). By-passing immunization. Human antibodies from V-gene libraries displayed on phage. J. Mol. Biol 222: 581-597.
65. Fishwild, D. M., O'Donnell, S. L., Bengoechea, T., Hudson, D. V., Harding, F., Bernhard, S. L., Jones, D., Kay, R. M., Higgins, K. M., Schramm, S. R., and Lonberg, N. (1996a). High-avidity human IgG kappa monoclonal antibodies from a novel strain of minilocus transgenic mice. Nat. Biotechnol. 14: 845-851.
66. Fishwild, D. M., O'Donnell, S. L., Bengoechea, T., Hudson, D. V., Harding, F., Bernhard, S. L., Jones, D., Kay, R. M., Higgins, K. M., Schramm, S. R, and Lonberg, N. (1996b). High-avidity human IgG kappa monoclonal antibodies from a novel strain of minilocus transgenic mice. Nat Biotechnol. 14: 845-851.
67. Lonberg, N. and Huszar, D. (1995b). Human antibodies from transgenic mice. Int Rev. Immunol. 13: 65-93.
68. Lonberg, N. and Huszar, D. (1995a). Human antibodies from transgenic mice. Int Rev. Immunol. 13: 65-93.
69. Lonberg, N., Taylor, L. D., Harding, FA., Trounstine, M., Higgins, KM., Schramm, S. R., Kuo, C. C., Mashayekh, R, Wymore, K., McCabe, J. G. (1994a). Antigen-specific human antibodies from mice comprising four distinct genetic modifications. Nature 368: 856-859.
70. Lonberg, N., Taylor, L. D., Harding, F. A., Trounstine, M., Higgins, K. M., Schramm, S. R., Kuo, C. C., Mashayekh, R., Wymore, K., McCabe, J. G. (1994b). Antigen-specific human antibodies from mice comprising four distinct genetic modifications. Nature 368: 856-859.
71. Morrison, S. L. (1994a). Immunology. Success in specification. Nature 368: 812-813.
72. Neuberger, M. (1996b). Generating high-avidity human Mabs in mice. Nat. Biotechnol. 14:826.
73. Huse, W. D., Sastry, L., Iverson, S. A., Kang, A. S., Alting-Mees, M., Burton, D. R., Benkovic, S. J., and Lerner, R. A. (1989). Generation of a large combinatorial library of the immunoglobulin repertoire in phage lambda. Science 246: 1275-1281.
74. Milstein, C. and Cuello, A. C. (1983). Hybrid hybridomas and their use in immunohistochemistry. Nature 305: 537-540.
75. Traunecker, A., Lanzavecchia, A., and Karjalainen, K. (1991). Bispecific single chain molecules (Janusins) target cytotoxic lymphocytes on HIV infected cells. EMBO J. 10: 3655-3659.
76. Suresh, M. R., Cuello, A. C., and Milstein, C. (1986). Advantages of bispecific hybridomas in one-step immunocytochemistry and immunoassays. Proc. Natl. Acad. Sci. U.S. A 83: 7989-7993.
77. Brennan, M., Davison, P. F., and Paulus, H. (1985). Preparation of bispecific antibodies by chemical recombination of monoclonal immunoglobulin G1 fragments. Science 229: 81-83.
78. Shalaby, M. R, Shepard, H. M., Presta, L., Rodrigues, M. L., Beverley, P. C., Feldmann, M., and Carter, P. (1992). Development of humanized bispecific antibodies reactive with cytotoxic lymphocytes and tumor cells overexpressing the HER2 protooncogene. J. Exp. Med. 175:217-225.
79. Kostelny, S. A., Cole, M. S., and Tso, J. Y. (1992). Formation of a bispecific antibody by the use of leucine zippers. J. Immunol. 148: 1547-1553.
80. Holliger, P., Prospero, T., and Winter, G. (1993). “Diabodies”: small bivalent and bispecific antibody fragments. Proc. Natl. Acad. Sci. U.S. A 90: 6444-6448.
81. Gruber, M., Schodin, B. A., Wilson, E. R., and Kranz, D. M. (1994). Efficient tumor cell lysis mediated by a bispecific single chain antibody expressed in Escherichia coli. J. Immunol. 152: 5368-5374.
82. Tutt, A., Stevenson, G. T., and Glennie, M. J. (1991). Trispecific F(ab′)3 derivatives that use cooperative signaling via the TCR/CD3 complex and CD2 to activate and redirect resting cytotoxic T cells. J. Immunol. 147: 60-69.
83. Caron, P. C., Laird, W., Co, M. S., Avdalovic, N. M., Queen, C., and Scheinberg, D. A. (1992). Engineered humanized dimeric forms of IgG are more effective antibodies. J. Exp. Med. 176:1191-1195.
84. Shopes, B. (1992). A genetically engineered human IgG mutant with enhanced cytolytic activity. J. Immunol. 148: 2918-2922.
85. Wolff, E. A., Schreiber, G. J., Cosand, W. L., and Raff, H. V. (1993). Monoclonal antibody homodimers: enhanced antitumor activity in nude mice. Cancer Res. 53: 2560-2565.

86. Stevenson, G. T., Pindar, A., and Slade, C. J. (1989). A chimeric antibody with dual Fc regions (bisFabFc) prepared by manipulations at the IgG hinge. Anticancer Drug Des 3: 219-230.

TABLE 1
Conserved amino acid residues in Spinster like 1 proteins of
Mouse (Mus musculus), Rat (Rattus norvegicus), and Human
(Homo sapiens).
conserved residues numbered as human Spinster like 1 amino acid
positions

| | | | | | | | | | | | | | |
|---|---|---|---|---|---|---|---|---|---|---|---|---|---|
| 1M | 2A | 3G | 4S | 5D | 6T | 7A | 8P | 9F | 10L | 11S | 12Q | 13A | 14D |
| 15D | 16P | 17D | 18D | 19G | 20P | 22P | 23G | 25P | 26G | 27L | 28P | 29G | 32G |
| 33N | 34P | 35K | 36S | 38E | 40E | 41V | 42P | 43D | 45E | 46G | 47L | 48Q | 49R |
| 50I | 51T | 52G | 53L | 54S | 56G | 58S | 60L | 61I | 62V | 64V | 65L | 66C | 67Y |
| 68I | 69N | 70L | 71L | 72N | 73Y | 74M | 75D | 76R | 77F | 78T | 79V | 80A | 81G |
| 82V | 83L | 85D | 86I | 87E | 88Q | 89F | 90F | 91N | 92I | 93G | 94D | 96S | 98G |
| 99L | 100I | 101Q | 102T | 103V | 104F | 105I | 106S | 107S | 108Y | 109M | 110V | 111L | 112A |
| 113P | 114V | 115F | 116G | 117Y | 118L | 119G | 120D | 121R | 122Y | 123N | 124R | 125K | 126Y |
| 128M | 129C | 130G | 131G | 132I | 133A | 134F | 135W | 136S | 137L | 138V | 139T | 140L | 141G |
| 142S | 143S | 144F | 145I | 146P | 148E | 149H | 150F | 151W | 152L | 153L | 155L | 156T | 157R |
| 158G | 160V | 161g | 162V | 163G | 164E | 165A | 166S | 167Y | 168S | 169T | 170I | 171A | 172P |
| 173T | 174L | 175I | 176A | 177D | 179F | 180V | 181A | 182D | 183Q | 184R | 185S | 186R | 187M |
| 188L | 189S | 190I | 191F | 192Y | 193F | 194A | 195I | 196P | 197V | 198G | 199S | 200G | 201L |
| 202g | 203Y | 204i | 205A | 206G | 207S | 208K | 209V | 210K | 214G | 215D | 216W | 217H | 218W |
| 219A | 220L | 221R | 222V | 223T | 224P | 225G | 226L | 227G | 228V | 230A | 231V | 232L | 233L |
| 234L | 235F | 236L | 237V | 238V | 240E | 241P | 242P | 243R | 244G | 245A | 246V | 247E | 248R |
| 249H | 250S | 253P | 254P | 255L | 257P | 258T | 259S | 260W | 261W | 262A | 263D | 264L | 266A |
| 267L | 268A | 269R | 271P | 272S | 273F | 274V | 275L | 276S | 277S | 278L | 279G | 280F | 281T |
| 283V | 284A | 285F | 286V | 287T | 288G | 289S | 290L | 291A | 292L | 293W | 294A | 295P | 296A |
| 297F | 298L | 299L | 300R | 301S | 302R | 303V | 304V | 305L | 306G | 307E | 308T | 309P | 310P |
| 311C | 312L | 313P | 314G | 315D | 316S | 317C | 318S | 319S | 320S | 321D | 322S | 323L | 324I |
| 325F | 326G | 327L | 328I | 329T | 330C | 331L | 332T | 333G | 334V | 335L | 336G | 337V | 338G |
| 339L | 340G | 342E | 343I | 344S | 345R | 346R | 347L | 348R | 351N | 352P | 353R | 354A | 355D |
| 356P | 357L | 358V | 359C | 360A | 362G | 363L | 364L | 365G | 366S | 368P | 369F | 370L | 371F |
| 372L | 374L | 375A | 376C | 377A | 378R | 379G | 380S | 381I | 382V | 383A | 384T | 385Y | 386I |
| 387F | 388I | 389F | 390I | 391G | 392E | 393T | 394L | 395L | 396S | 397M | 398N | 399W | 400A |
| 401I | 402V | 403A | 404D | 405I | 406L | 407L | 408Y | 409V | 410V | 411I | 412P | 413T | 414R |
| 415R | 416S | 417T | 418A | 419E | 420A | 421F | 422Q | 423I | 424V | 425L | 426S | 427H | 428L |
| 429L | 430G | 431D | 432A | 433G | 434S | 435P | 436Y | 437L | 438I | 439G | 440L | 441I | 442S |
| 443D | 444R | 445L | 446R | 447R | 449W | 450P | 451P | 452S | 453F | 454L | 455S | 456E | 457F |
| 458R | 459A | 460L | 461Q | 462F | 463S | 464L | 465M | 466L | 467C | 468A | 469F | 470V | 471G |
| 472A | 473L | 474G | 475G | 476A | 477A | 478F | 479L | 480G | 481T | 482A | 484F | 485I | 486E |
| 488D | 489R | 490R | 491R | 492A | 493Q | 494L | 495H | 496V | 497Q | 498G | 499L | 500L | 501H |
| 502E | 507D | 508D | 510I | 511V | 512V | 513P | 514Q | 515R | 516G | 517R | 518S | 519T | 520R |
| 521V | 522P | 523V | 525S | 526V | 527L | 528I | 213A | | | | | | |

Explanation of amino acid single letter code:
A = Ala
C = Cys
D = Asp
E = Glu
F = Phe
G = Gly
H = His
I = Ile
K = Lys
L = Leu
M= Met
N = Asn
P= Pro
Q = Gln
R = Arg
S = Ser
T = Thr
V = Val
W = Trp
Y = Tyr

TABLE 2
Conserved amino acid residues in Spinster like 1 proteins of
Mouse (Mus musculus), Rat (Rattus norvegicus), Human (Homo
sapiens), and Zebrafish (Danio rerio).
conserved residues numbered as human Spinster like 1 amino acid
positions

| | | | | | | | | | | | | | |
|---|---|---|---|---|---|---|---|---|---|---|---|---|---|
| 2A | 5D | 8P | 9F | 14D | 19G | 20P | 26G | 28P | 34P | 38E | 40E | 42P | 52G |
| 62V | 64V | 65L | 66C | 67Y | 68I | 69N | 70L | 71L | 72N | 73Y | 74M | 75D | 76R |
| 77F | 78T | 79V | 80A | 81G | 82V | 83L | 85D | 86I | 87E | 89F | 90F | 92I | 93G |
| 94D | 98G | 99L | 101Q | 102T | 103V | 104F | 105I | 107S | 108Y | 109M | 111L | 112A | 113P |
| 115F | 116G | 117Y | 118L | 119G | 120D | 121R | 122Y | 123N | 124R | 125K | 128M | 129C | 131G |
| 132I | 134F | 135W | 138V | 139T | 140L | 142S | 143S | 144F | 145I | 149H | 150F | 151W | 153L |
| 155L | 156T | 157R | 158G | 160V | 161G | 162V | 163G | 164E | 165A | 166S | 167Y | 168S | 169T |
| 170I | 171A | 172P | 173T | 175I | 176A | 177D | 179F | 180V | 184R | 187M | 188L | 189S | 190I |
| 191F | 192Y | 193F | 194A | 195I | 196P | 197V | 198G | 199S | 200G | 202G | 203Y | 204I | 206G |
| 207S | 208K | 209V | 213A | 215D | 216W | 217H | 218W | 219A | 220L | 221R | 222V | 223T | 224P |
| 225G | 226L | 227G | 230A | 231V | 233L | 234L | 236L | 237V | 238V | 240E | 241P | 243R | 244G |
| 245A | 247E | 249H | 255L | 258T | 259S | 260W | 262A | 263D | 266A | 267L | 269R | 271P | 272S |
| 273F | 275L | 276S | 279F | 280F | 281T | 283V | 284A | 285F | 286V | 287T | 288G | 289S | 290L |
| 291A | 292L | 293W | 294A | 295P | 296A | 297F | 298L | 300R | 303V | 306G | 310P | 311C | 317C |
| 320S | 321D | 322S | 323L | 324I | 325F | 326G | 328I | 329T | 332T | 333G | 335L | 336G | 337V |
| 340G | 344S | 347L | 348R | 352P | 353R | 354A | 355D | 356P | 357L | 358V | 359C | 360A | 362G |
| 363L | 364L | 368P | 369F | 370L | 372L | 377A | 380S | 382V | 383A | 384T | 385Y | 387F | 388I |
| 389F | 391G | 392E | 393T | 395L | 396S | 397M | 398N | 399W | 400A | 401I | 402V | 403A | 404D |
| 405I | 406L | 407L | 408Y | 409V | 410V | 411I | 412P | 413T | 414R | 415R | 416S | 417T | 418A |
| 419E | 420A | 421F | 422Q | 423I | 424V | 425L | 426S | 427H | 428L | 429L | 430G | 431D | 432A |
| 434S | 435P | 436Y | 437L | 438I | 439G | 442S | 443D | 452S | 456E | 457F | 458R | 460L | 461Q |
| 463S | 464L | 466L | 467C | 469F | 470V | 474G | 475G | 476A | 478F | 479L | 481T | 482A | 484F |
| 485I | 486E | 488D | 489R | 492A | 496V | 507D | 510I | 511V | 512V | 513P | 516G | 517R | 518S |
| 519T | 521V | 523V | 525S | 526V | 527L | 528I | 136S | | | | | | |

Explanation of amino acid single letter code:
A = Ala
C = Cys
D = Asp
E = Glu
F = Phe
G = Gly
H = His
I = Ile
K = Lys
L = Leu
M = Met
N = Asn
P = Pro
Q = Gln
R = Arg
S = Ser
T = Thr
V = Val
W = Trp
Y = Tyr

TABLE 3
Conserved amino acid residues in Spinster like 1 proteins of
Mouse (Mus musculus), Rat (Rattus norvegicus), Human (Homo
sapiens), Zebrafish (Danio rerio), and Fugu (Takifugus rubriens).
conserved residues numbered as human Spinster like 1 amino acid
positions

| | | | | | | | | | | | | | |
|---|---|---|---|---|---|---|---|---|---|---|---|---|---|
| 2A | 5D | 8P | 9F | 14D | 19G | 20P | 26G | 28P | 34P | 38E | 40E | 42P | 52G |
| 62V | 64V | 65L | 66C | 67Y | 68I | 69N | 70L | 71L | 72N | 73Y | 74M | 75D | 76R |
| 77F | 78T | 79V | 80A | 81G | 82V | 83L | 85D | 86I | 87E | 89F | 90F | 92I | 93G |
| 94D | 98G | 103V | 104F | 105I | 107S | 108Y | 109M | 111L | 112A | 113P | 115F | 116G | 117Y |
| 118L | 119G | 120D | 121R | 122Y | 123N | 124R | 125K | 128M | 131G | 131G | 134F | 135W | 136S |
| 138V | 139T | 140L | 142S | 143S | 149H | 150F | 151W | 153L | 156T | 157R | 158G | 160V | 161G |
| 162V | 163G | 164E | 165A | 166S | 167Y | 168S | 169T | 170I | 171A | 172P | 173T | 175I | 176A |
| 177D | 180V | 184R | 187M | 188L | 189S | 191F | 192Y | 193F | 194A | 195I | 196P | 197V | 198G |
| 199S | 200G | 202G | 203Y | 204I | 206G | 207S | 209V | 213A | 215D | 216W | 217H | 218W | 219A |
| 220L | 221R | 222V | 223T | 224P | 225G | 226L | 227G | 230A | 231V | 233L | 234L | 236L | 237V |
| 238V | 240E | 241P | 243R | 244G | 245A | 247E | 258T | 260W | 263D | 266A | 267L | 269R | 272S |
| 273F | 275L | 276S | 279G | 280F | 281T | 283V | 284A | 285F | 286V | 287T | 288G | 289S | 290L |
| 291A | 292L | 293W | 294A | 295P | 297F | 298L | 300R | 303V | 306G | 310P | 311C | 317C | 320S |
| 321D | 322S | 323L | 325F | 326G | 328I | 329T | 332T | 333G | 335L | 336G | 337V | 340G | 344S |
| 347L | 348R | 353R | 354A | 355D | 356P | 357L | 358V | 359C | 360A | 362G | 363L | 364L | 368P |
| 369F | 370L | 372L | 377A | 380S | 382V | 383A | 384T | 385Y | 387F | 388I | 389F | 391G | 392E |
| 393T | 395L | 396S | 397M | 398N | 399W | 400A | 401I | 402V | 403A | 404D | 405I | 406L | 407L |
| 408Y | 409V | 410V | 412P | 413T | 414R | 415R | 417T | 418A | 419E | 420A | 422Q | 423I | 426S |
| 427H | 428L | 429L | 430G | 431D | 432A | 434S | 435P | 436Y | 437L | 438I | 439G | 442S | 443D |
| 452S | 457F | 458R | 460L | 461Q | 463S | 464L | 466L | 467C | 469F | 470V | 474G | 475G | 476A |
| 478F | 479L | 481T | 482A | 484F | 478F | 485I | 486E | 488D | 489R | 492A | 507D | 510I | 511V |
| 512V | 513P | 516G | 517R | 518S | 519T | 521V | 523V | 525S | 526V | 527L | 528I | | |

Explanation of amino acid single letter code:
A = Ala
C = Cys
D = Asp
E = Glu
F = Phe
G = Gly
H = His
I = Ile
K = Lys
L = Leu
M = Met
N = Asn
P = Pro
Q = Gln
R = Arg
S = Ser
T = Thr
V = Val
W = Trp
Y = Tyr

1 to 223 (Canceled)

224. An isolated protein having at least 63%, 65%, 70%, 75%, 80%, 85%, 90%, 95%, 98%, or 99% amino acid identity compared to the mouse Spinl1 or the human Spinl1 protein according to SEQ ID NO:3 and SEQ ID NO:7, respectively, or an isolated fragment of such protein comprising at least 6, 7, 8, 9, 10, 15, 20, 25, 30, 35, 40, 50, 60, 70, 80, 90, 100, 150, 200, 250, 300, 350, 400, 450, 460, 470, 480, 490, 500, 510, 520, 521, 522, 523, 524, 525, 526, or 527 contiguous amino acids having said percentages of amino acid identity compared to the corresponding amino acids in SEQ ID NO:3 and SEQ ID NO:7, wherein said protein or fragment of such protein comprises an amino acid or an amino acid sequence which corresponds to:
(a) a mutation in the mouse Spinl1 protein as defined above which, if encoded by the mouse Spinl1 gene and present in the genome of all or essentially all cells of a mouse in a homozygous manner, results in a phenotype associated with an alteration in fat metabolism compared to the corresponding wild-type animal;
(b) a mutation in the mouse Spinl1 protein as defined above which, if encoded by the mouse Spinl1 gene and present in the genome of all or essentially all cells of a mouse in a homozygous manner, results in a phenotype associated with an alteration in serum leptin level and/or an alteration in leptin sensitivity compared to the corresponding wild-type animal; or
(c) a mutation in the mouse Spinl1 protein as defined above which, if encoded by the mouse Spinl1 gene and present in the genome of all or essentially all cells of a mouse in a homozygous manner, results in a phenotype associated with an alteration in plasma insulin level and/or an alteration in insulin sensitivity compared to the corresponding wild-type animal.

225. The isolated protein or protein fragment according to claim 224, wherein said protein represents an orthologue of the mouse Spinl1 or the human Spinl1 protein, preferably a vertebrate orthologue, in particular an orthologue wherein said vertebrate is Danio rerio or Takifugus rubiens, or a mammalian orthologue, in particular an orthologue wherein said mammal is selected from the group consisting of a rat, rabbit, hamster, dog, cat, sheep, bovine, and a horse.

226. The isolated protein or protein fragment according to claim 224, wherein said alteration results in a loss of function phenotype.

227. The isolated protein or protein fragment according to claim 224, wherein said alteration recited in (a) is an alteration in fat storage, particularly a reduction in fat storage, and/or an alteration in liver function.

228. The isolated protein or protein fragment according to claim 227 wherein said reduction in fat storage is a reduction in the mass of fat.

229. The isolated protein or protein fragment according to claim 224, wherein said alteration recited in (a) is an alteration selected from the group consisting of a serum elevation of components of the cholesterol metabolism, particularly cholesterol, cholinesterase, high density lipoprotein, low density lipoprotein; a size reduction of white adipocytes; an absence of intracellular fat vacuoles; a serum elevation of liver enzymes, particularly alkaline phosphatase, glutamic-oxaloacetic transaminase, glutamate pyruvate transaminase, and lactate dehydrogenase; a serum reduction of lactate; and an accumulation of electron dense material in hepatocytes.

230. The isolated protein or protein fragment according to claim 224, wherein said alteration recited in (b) is an alteration selected from the group consisting of a serum reduction of leptin and a plasma reduction of glucose.

231. The isolated protein or protein fragment according to claim 224, wherein said alteration recited in (c) is a plasma reduction of insulin, or an increase in insulin sensitivity.

232. The isolated protein or protein fragment according to claim 224, wherein said alteration results in a thriving deficit, particularly a reduction in body weight and body length, optionally associated with hypoglycemia.

233. The isolated protein or protein fragment according to claim 224, wherein said alteration results in a gain of function phenotype.

234. The isolated protein or protein fragment according to claim 224, wherein said mutation results in a deletion or substitution by another amino acid of an amino acid of said mouse Spinl1 protein, or an insertion of additional amino acids not normally present in the amino acid sequence of said mouse Spinl1 protein.

235. The isolated protein or protein fragment according to claim 234, wherein the substitution of said amino acid of said mouse Spinl1 protein by another amino acid is a non-conservative substitution.

236. The isolated protein or protein fragment according to claim 234, wherein the amino acid of said mouse Spinl1 protein that is deleted or substituted is Tyr108.

237. The isolated protein or protein fragment according to claim 236, wherein the substitution at position Tyr108 is one of the following substitutions:
a) Tyr→acidic amino acid such as Glu or Asp;
b) Tyr→basic amino acid, such as His, Arg or Lys;
c) Tyr→aliphatic hydroxyl side chain amino acid, such as Ser or Thr;
d) Tyr→amide side chain amino acid, such as Asn or Gln;
e) Tyr→sulfur containing side chain amino acid, such as Cys or Met;
f) Tyr→aromatic side chain amino acid, such as Phe, Trp;
g) Tyr→Gly, Val or Pro; and
h) Tyr→Ala, Leu or Ile.

238. The isolated protein or protein fragment according to claim 237, wherein the substitution at position Tyr108 is a substitution of tyrosine by histidine.

239. An isolated protein having the amino acid sequence as set forth in SEQ ID NO:4 or SEQ ID NO:8, or an isolated fragment of such protein comprising at least 6, 7, 8, 9, 20, 15, 20, 25, 30, 35, 40, 50, 60, 70, 80, 90, 100, 150, 200, 250, 300, 350, 400, 450, 460, 470, 480, 490, 500, 510, 520, 521, 522, 523, 524, 525, 526, or 527 contiguous amino acids of said amino acid sequence, said amino acid sequence comprising an amino acid corresponding to His108.

240. A fusion protein comprising a protein or protein fragment according to claim 224 fused to another protein or protein fragment not having any of the percentages of amino acid sequence identity to any corresponding amino acids in SEQ ID NO:3 and SEQ ID NO:7 as defined in claim 224.

241. The fusion protein of claim 240, wherein said other protein is a protein unrelated to the mouse Spinl1 or the human Spinl1 protein according to SEQ ID NO:3 and SEQ ID NO:7, respectively.

242. An isolated nucleic acid encoding a protein or a fragment of such protein or a fusion protein according to claim 224, or an isolated nucleic acid, which is complementary thereto.

243. An isolated nucleic acid having the nucleotide sequence set forth in SEQ ID NO:2 and SEQ ID NO:6, or an isolated nucleic acid which is complementary thereto.

244. An episomal element comprising a nucleic acid as defined in claim 242.

245. The episomal element according to claim 244, wherein said episomal element is selected from a plasmid, a cosmid, a bacterial phage nucleic acid, or a viral nucleic acid.

246. A vector comprising a nucleic acid molecule encoding the protein or fragment of such a protein or the fusion protein according to claim 224.

247. A host cell transfected with the nucleic acid of claim 242.

248. A host cell transfected with the episomal element of claim 244.

249. A host cell transfected with the vector of claim 246.

250. A method of producing a mutant Spinl1 protein comprising culturing a host cell according to claim 247 in a suitable medium under conditions such that the protein is expressed, and harvesting the cells or the medium.

251. An antisense nucleic acid comprising a nucleotide sequence which is complementary to a part of an mRNA encoding
(i) the mouse Spinl1 or the human Spinl1 protein according to SEQ ID NO:3 and SEQ ID NO:7, respectively, or an orthologue thereof having at least 63%, 65%, 70%, 75%, 80%, 85%, 90%, 95%, 98%, or 99% amino acid identity compared to the mouse Spinl1 or the human Spinl1 protein according to SEQ ID NO:3 and SEQ ID NO:7, respectively; or
(ii) a protein which affects the expression or activity of the mouse Spinl1 or the human Spinl1 protein according to SEQ ID NO:3 and SEQ ID NO:7, respectively, or an orthologue thereof having at least 63%, 65%, 70%, 75%, 80%, 85%, 90%, 95%, 98%, or 99% amino acid identity compared to the mouse Spinl1 or the human Spinl1 protein according to SEQ ID NO:3 and SEQ ID NO:7, respectively.

252. The antisense nucleic acid of claim 251, wherein said antisense nucleic acid is capable of hybridizing to said mRNA via said complementary nucleotide sequence under physiological conditions, or under conditions of high stringency, preferably under hybridization conditions of a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C., furthermore preferably under hybridization conditions of a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 65° C.

253. A host cell transformed with an antisense nucleic acid according to claim 251.

254. A short interfering RNA (siRNA) comprising a double stranded nucleotide sequence wherein one strand is complementary to an at least 19, 20, 21, 22, 23, 24, or 25 nucleotide long segment of an mRNA encoding
(i) the mouse Spinl1 or the human Spinl1 protein according to SEQ ID NO:3 and SEQ ID NO:7, respectively, or an orthologue thereof having at least 63%, 65%, 70%, 75%, 80%, 85%, 90%, 95%, 98%, or 99% amino acid identity compared to the mouse Spinl1 or the human Spinl1 protein according to SEQ ID NO:3 and SEQ ID NO:7, respectively;
(ii) a protein which affects the expression or activity of the mouse Spinl1 or the human Spinl1 protein according to SEQ ID NO:3 and SEQ ID NO:7, respectively, or an orthologue thereof having at least 63%, 65%, 70%, 75%, 80%, 85%, 90%, 95%, 98%, or 99% amino acid identity compared to the mouse Spinl1 or the human Spinl1 protein according to SEQ ID NO:3 and SEQ ID NO:7, respectively;
(iii) the mouse Spinl1 protein according to SEQ ID NO:3, said segment having a sequence selected from the group of sequences consisting of SEQ ID NO:43, SEQ ID NO:44, SEQ ID NO:45, SEQ ID NO:46, SEQ ID NO:47, and SEQ ID NO:48; or
(iv) the human Spinl1 protein according to SEQ ID NO:7, said segment having a sequence which represents the sequence in SEQ ID NO:5 (human Spinl1 mRNA wild type) corresponding to SEQ ID NO:43, SEQ ID NO:44, SEQ ID NO:45, SEQ ID NO:46, SEQ ID NO:47, or SEQ ID NO:48.

255. The siRNA according to claim 254, wherein said segment includes sequences from the 5′ untranslated (UT) region, the open reading frame (ORF), or the 3′ UT region of said mRNA.

256. A host cell transformed with an siRNA according to claim 254.

257. A non-human vertebrate animal comprising in the genome of at least some of its cells an allele of a gene encoding a protein having at least 63%, 65%, 70%, 75%, 80%, 85%, 90%, 95%, 98%, or 99% amino acid identity compared to the mouse Spinl1 or the human Spinl1 protein according to SEQ ID NO:3 and SEQ ID NO:7, respectively, said allele comprising a mutation which, if present in the genome of all or essentially all cells of said animal in a homozygous manner, results in
(a) a phenotype associated with an alteration in fat metabolism compared to the corresponding wild-type animal;
(b) a phenotype associated with an alteration in serum leptin level and/or an alteration in leptin sensitivity compared to the corresponding wild-type animal; or
(c) a phenotype associated with an alteration in plasma insulin level and/or an alteration in insulin sensitivity compared to the corresponding wild-type animal.

258. A non-human vertebrate animal comprising in the genome of at least some of its cells an allele of a gene coding for a protein which affects expression or activity of the Spinl protein of said animal, said allele comprising a mutation which, if present in the genome of all or essentially all cells of said animal in a homozygous manner, results in a phenotype associated with an alteration in fat metabolism compared to the corresponding wild-type animal.

259. The animal according to claim 257, wherein said alteration recited in (a) results in a loss of function phenotype.

260. The animal according to claim 257, wherein said alteration recited in (a) is an alteration in fat storage, particularly a reduction in fat storage, and/or an alteration in liver function.

261. The animal according to claim 260 wherein said reduction in fat storage is a reduction in the mass of fat.

262. The animal according to claim 257, wherein said alteration recited in (a) is an alteration selected from the group consisting of a serum elevation of components of the cholesterol metabolism, particularly cholesterol, cholinesterase, high density lipoprotein, low density lipoprotein; a size reduction of white adipocytes; an absence of intracellular fat vacuoles; a serum elevation of liver enzymes, particularly alkaline phosphatase, glutamic-oxaloacetic transaminase, glutamate pyruvate transaminase, and lactate dehydrogenase; a serum reduction of lactate; and an accumulation of electron dense material in hepatocytes.

263. The animal according to claim 257, wherein said alteration recited in (b) is an alteration selected from the group consisting of a serum reduction of leptin and a plasma reduction of glucose.

264. The animal according to claim 257, wherein said alteration recited in (c) is a plasma reduction of insulin, or an increase in insulin sensitivity.

265. The animal according to claim 257, wherein said alteration results in a thriving deficit, particularly a reduction in body weight and body length, optionally associated with hypoglycemia.

266. The animal according to claim 257, wherein said alteration results in a gain of function phenotype.

267. The animal according to claim 257, wherein said gene encodes a protein which is an orthologue of SEQ ID NO:3 and SEQ ID NO:7 with respect to said animal.

268. The animal according to claim 257 wherein said gene encodes a protein according to claim 224.

269. The animal according to claim 268, wherein said gene encodes a protein having the amino acids of SEQ ID NO:4 or SEQ ID NO:8.

270. The animal according to claim 257, wherein said animal is a transgenic animal.

271. The animal according to any one of claim 257, wherein said cells are germ cells.

272. The animal according to claim 257, wherein said cells are somatic cells.

273. The animal according to claim 257, wherein said genome of said cells is homozygous in respect of said allele.

274. The animal according to claim 257, wherein said animal is a mammalian animal, preferably a rodent.

275. The animal according to claim 274, wherein said animal is selected from the group consisting of a mouse, rat, rabbit, hamster, dog, cat, sheep, bovine, and horse.

276. A method for
(a) the identification of a protein or nucleic acid diagnostic marker for an alteration in fat metabolism, or for studying the molecular mechanisms of, or physiological processes associated with an alteration in fat metabolism; or for the identification and testing of an agent useful in the prevention, amelioration, or treatment of a medical condition associated with an alteration in fat metabolism;
(b) the identification of a protein or nucleic acid diagnostic marker for an alteration in serum leptin level and/or an alteration in leptin sensitivity or for studying the molecular mechanisms of, or physiological processes associated with an alteration in serum leptin level and/or an alteration in leptin sensitivity; or for the identification and testing of an agent useful in the prevention, amelioration, or treatment of a medical condition associated with an alteration in serum leptin level and/or an alteration in leptin sensitivity; or
(c) the identification of a protein or nucleic acid diagnostic marker for an alteration in plasma insulin level and/or an alteration in insulin sensitivity or for studying the molecular mechanisms of, or physiological processes associated with an alteration in plasma insulin level and/or an alteration in insulin sensitivity; or for the identification and testing of an agent useful in the prevention, amelioration, or treatment of a medical condition associated with an alteration in plasma insulin level and/or an alteration in insulin sensitivity;
the method comprising subjecting an organ or tissue sample of the non-human vertebrate animal according to claim 257 to procedures of proteomics or gene expression analysis or administering said agent to the non-human vertebrate animal of claim 257 and measuring or monitoring a phenotypic parameter in said animal.

277. A method for studying the molecular mechanisms of, or physiological processes associated with, or medical condition associated with, or affected by, reduced activity or undesirable activity of endogenous Spinl1, or reduced expression, reduced production or undesirable production of endogenous Spinl1; or for the identification and testing of an agent useful in the prevention, amelioration, or treatment of these conditions comprising subjecting an organ or tissue sample of the non-human vertebrate animal according to claim 257 to procedures of proteomics or gene expression analysis or administering said agent to the non-human vertebrate animal of claim 257 and measuring or monitoring a phenotypic parameter in said animal.

278. The method of claim 276, wherein said medical condition recited in (a) is selected from the group consisting of obesity; obesity and diabetes, particularly type II diabetes; and diabetes, particularly type II diabetes.

279. The method of claim 277, wherein said medical condition is selected from the group consisting of obesity; obesity and diabetes, particularly type II diabetes; and diabetes, particularly type II diabetes.

280. The method of claim 276, wherein said medical condition recited in (b) is selected from the group consisting of obesity; obesity and diabetes, particularly type II diabetes; diabetes, particularly type II diabetes; chronic kidney disease; coronary atherosclerosis; rheumatoid arthritis; osteoarthritis; anorexia nervosa; rheumatoid arthritis; osteoarthritis; osteoporosis; gastrointestinal diseases, in particular gastric disease, peptic ulcer, intestinal bowel disease, in particular Crohn's disease or ulcerative colitis; cardiovascular diseases, in particular cardiac hypertrophy; obstructive sleep apnea; maculopathy, in particular age-related maculopathy (ARM) or degeneration (ARMD); and prostate cancer.

281. The method of claim 276, wherein said medical condition recited in (c) is selected from the group consisting of obesity; obesity and diabetes, particularly type II diabetes; diabetes, particularly type II diabetes; chronic kidney disease; coronary atherosclerosis; rheumatoid arthritis; osteoarthritis; anorexia nervosa; rheumatoid arthritis; osteoarthritis; osteoporosis; gastrointestinal diseases, in particular gastric disease, peptic ulcer, intestinal bowel disease, in particular Crohn's disease or ulcerative colitis; cardiovascular diseases, in particular cardiac hypertrophy; obstructive sleep apnea; maculopathy, in particular age-related maculopathy (ARM) or degeneration (ARMD); and prostate cancer.

282. The method of claim 276, wherein said agent is selected from the group consisting of a small molecule drug, a (poly)peptide, or a nucleic acid.

283. The method of claim 277, wherein said agent is selected from the group consisting of a small molecule drug, a (poly)peptide, or a nucleic acid.

284. A pharmaceutical composition comprising a protein or protein fragment according to claim 224 and a pharmaceutically acceptable carrier.

285. A pharmaceutical composition comprising a nucleic acid according to claim 242 and a pharmaceutically acceptable carrier.

286. A pharmaceutical composition comprising an episomal element according to claim 244 and a pharmaceutically acceptable carrier.

287. A pharmaceutical composition comprising a vector according to claim 246 and a pharmaceutically acceptable carrier.

288. A pharmaceutical composition comprising an antisense nucleic acid according to claim 251 and a pharmaceutically acceptable carrier.

289. A pharmaceutical composition comprising an siRNA according to claim 254 and a pharmaceutically acceptable carrier.

290. A pharmaceutical composition comprising:
(a) an isolated protein having the sequence of the human Spinl1 protein of a human subject known not to have a medical condition associated with
(i) an alteration in fat metabolism;
(ii) an alteration in serum leptin level and/or an alteration in leptin sensitivity; or
(iii) an alteration in plasma insulin level and/or an alteration in insulin sensitivity, preferably the protein according to SEQ ID NO:7 or an allelic variant thereof, or a fragment of such protein or allelic variant which is effective in treating said medical condition; or
(b) a protein, allelic variant, or fragment as defined in (a) fused to another protein unrelated to the mouse Spinl1 or the human Spinl1 protein according to SEQ ID NO:3 and SEQ ID NO:7, respectively; or
(c) an orthologue having at least 63%, 65%, 70%, 75%, 80%, 85%, 90%, 95%, 98%, or 99% amino acid identity compared to the Spinl1 protein, allelic variant, or fragment as defined in (a); or
(d) an aptamer specifically recognizing an epitope comprised within the human Spinl1 protein of a human subject known not to have a medical condition associated with
(i) an alteration in fat metabolism;
(ii) an alteration in serum leptin level and/or an alteration in leptin sensitivity; or
(iii) an alteration in plasma insulin level and/or an alteration in insulin sensitivity, preferably the protein according to SEQ ID NO:7 or an allelic variant thereof; or
(e) an anticalin specifically recognizing an epitope comprised within the human Spinl1 protein of a human subject known not to have a medical condition associated with
(i) an alteration in fat metabolism;
(ii) an alteration in serum leptin level and/or an alteration in leptin sensitivity; or
(iii) an alteration in plasma insulin level and/or an alteration in insulin sensitivity, preferably the protein according to SEQ ID NO:7 or an allelic variant thereof; or
(f) an antibody specifically recognizing an epitope comprised within the human Spinl1 protein of a human subject known not to have a medical condition associated with
(i) an alteration in fat metabolism;
(ii) an alteration in serum leptin level and/or an alteration in leptin sensitivity; or
(iii) an alteration in plasma insulin level and/or an alteration in insulin sensitivity preferably the protein according to SEQ ID NO:7 or an allelic variant thereof; or
(g) a (poly)peptide or a small molecule drug, which modulates the activity of the human Spinl1 protein of a human subject known not to have said medical condition, preferably the human Spinl1 protein according to SEQ ID NO:7 or an allelic variant thereof;
and a pharmaceutically acceptable carrier.

291. The pharmaceutical composition of claim 290, wherein said medical condition recited in (a)(i), (d)(i), (e)(i), or (f)(i), is selected from the group consisting of obesity;
obesity and diabetes, particularly type II diabetes; and diabetes, particularly type II diabetes.

292. The pharmaceutical composition of claim 290, wherein said medical condition recited in (a)(ii), (d)(ii), (e)(ii), or (f)(ii) is selected from the group consisting of obesity; obesity and diabetes, particularly type II diabetes; diabetes, particularly type II diabetes; chronic kidney disease; coronary atherosclerosis; anorexia nervosa; rheumatoid arthritis; osteoarthritis; osteoporosis; gastrointestinal diseases, in particular gastric disease, peptic ulcer, intestinal bowel disease, in particular Crohn's disease or ulcerative colitis; cardiovascular diseases, in particular cardiac hypertrophy; obstructive sleep apnea; maculopathy, in particular age-related maculopathy (ARM) or degeneration (ARMD); and prostate cancer.

293. The pharmaceutical composition of claim 290, wherein said medical condition recited in (a)(iii), (d)(iii), (e)(iii), or (f)(iii), is selected from the group consisting of obesity; obesity and diabetes, particularly type II diabetes; diabetes, particularly type II diabetes; chronic kidney disease; coronary atherosclerosis; anorexia nervosa; rheumatoid arthritis; osteoarthritis; osteoporosis; gastrointestinal diseases, in particular gastric disease, peptic ulcer, intestinal bowel disease, in particular Crohn's disease or ulcerative colitis; cardiovascular diseases, in particular cardiac hypertrophy; obstructive sleep apnea; maculopathy, in particular age-related maculopathy (ARM) or degeneration (ARMD); and prostate cancer.

294. A method of gene therapy comprising delivering to cells in a human subject suffering from or known to be at risk of developing a medical condition associated with an alteration in fat metabolism; or with an alteration in serum leptin level and/or an alteration in leptin sensitivity; or with an alteration in plasma insulin level and/or an alteration in insulin sensitivity a DNA construct comprising
(a) a sequence of an allele of the Spinl1 gene encoding the human Spinl1 protein of a human subject known not to have a medical condition associated with said alterations, preferably the protein according to SEQ ID NO:7 or an allelic variant thereof; or a sequence encoding a protein having at least 63%, 65%, 70%, 75%, 80%, 85%, 90%, 95%, 98%, or 99% amino acid identity compared to the mouse Spinl1 or the human Spinl1 protein according to SEQ ID NO:3 and SEQ ID NO:7, respectively; or
(b) a sequence encoding the human Spinl1 protein of a human subject known not to have a medical condition associated with an alteration in fat metabolism, preferably the protein according to SEQ ID NO:7 or an allelic variant thereof; or a sequence encoding a protein having at least 63%, 65%, 70%, 75%, 80%, 85%, 90%, 95%, 98%, or 99% amino acid identity compared to the mouse Spinl1 or the human Spinl1 protein according to SEQ ID NO:3 and SEQ ID NO:7, respectively; or
(c) a sequence encoding an antisense nucleic acid according to claim 251.

295. A method of gene therapy comprising delivering to cells in a human subject suffering from or known to be at risk of developing a medical condition associated with an alteration in fat metabolism or with an alteration in serum leptin level and/or an alteration in leptin sensitivity or with an alteration in plasma insulin level and/or an alteration in insulin sensitivity a DNA construct comprising a sequence encoding an siRNA according to claim 254.

296. A method of gene therapy comprising delivering to cells in a human subject suffering from or known to be at risk of developing a medical condition associated with an alteration in fat metabolism or with an alteration in serum leptin level and/or an alteration in leptin sensitivity or with an alteration in plasma insulin level and/or an alteration in insulin sensitivity a DNA construct comprising a sequence encoding a protein according to claim 224.

297. The method of claim 294, wherein the DNA construct is a viral vector.

298. The method of claim 294, wherein said DNA construct is capable of directing expression of said antisense nucleic acid or said siRNA.

299. The method of claim 298, wherein said expression is transient.

300. The method of claim 294, wherein the DNA construct is capable of being stably integrated into the genome of said cells.

301. The method of claim 294, wherein said sequence of an allele of the Spinl1 gene comprises coding sequences of said gene.

302. The method of claim 294, wherein said sequence of an allele of the Spinl1 gene comprises non-coding sequences of said gene.

303. A method of preventing, treating, or ameliorating a medical condition in a human subject associated with
(a) an alteration in fat metabolism;
(b) an alteration in serum leptin level and/or an alteration in leptin sensitivity; or
(c) an alteration in plasma insulin level and/or an alteration in insulin sensitivity said method comprising administering to said human subject a pharmaceutical composition comprising an agent capable of modulating Spinl1 activity in said human subject.

304. The method of claim 303, wherein said pharmaceutical composition is a pharmaceutical composition according to claim 284.

305. The method of claim 303, wherein said pharmaceutical composition is a pharmaceutical composition according to claim 285.

306. The method of claim 303, wherein said pharmaceutical composition is a pharmaceutical composition according to claim 286.

307. The method of claim 303, wherein said pharmaceutical composition is a pharmaceutical composition according to claim 287.

308. The method of claim 303, wherein said pharmaceutical composition is a pharmaceutical composition according to claim 288.

309. The method of claim 303, wherein said pharmaceutical composition is a pharmaceutical composition according to claim 289.

310. The method of claim 303, wherein said pharmaceutical composition is a pharmaceutical composition according to claim 290.

311. The method according to claim 303, wherein said medical condition recited in (a) is selected from the group consisting of obesity; obesity and diabetes, particularly type II diabetes; and diabetes, particularly type II diabetes.

312. The method according to claim 303, wherein said medical condition recited in (b) is selected from the group consisting of obesity; obesity and diabetes, particularly type II diabetes; diabetes, particularly type II diabetes; chronic kidney disease; coronary atherosclerosis; anorexia nervosa; rheumatoid arthritis; osteoarthritis; osteoporosis; gastrointestinal diseases, in particular gastric disease, peptic ulcer, intestinal bowel disease, in particular Crohn's disease or ulcerative colitis; cardiovascular diseases, in particular cardiac hypertrophy; obstructive sleep apnea; maculopathy, in particular age-related maculopathy (ARM) or degeneration (ARMD); and prostate cancer.

313. The method according to claim 303, wherein said medical condition recited in (c) is selected from the group consisting of obesity; obesity and diabetes, particularly type II diabetes; diabetes, particularly type II diabetes; chronic kidney disease; coronary atherosclerosis; anorexia nervosa; rheumatoid arthritis; osteoarthritis; osteoporosis; gastrointestinal diseases, in particular gastric disease, peptic ulcer, intestinal bowel disease, in particular Crohn's disease or ulcerative colitis; cardiovascular diseases, in particular cardiac hypertrophy; obstructive sleep apnea; maculopathy, in particular age-related maculopathy (ARM) or degeneration (ARMD); and prostate cancer.

314. The method of claim 303, wherein the pharmaceutical composition or the agent is capable of increasing insulin sensitivity in said human subject, and wherein the medical condition is selected from obesity; obesity and diabetes, particularly type II diabetes; and diabetes, particularly type II diabetes.

315. The method according to claim 314, wherein the agent or the pharmaceutical composition is administered in conjunction with one or more compounds useful in the treatment of type II diabetes.

316. The method according to claim 315, wherein the compound(s) is (are) selected from the group consisting of a sulfonylurea, any other insulin secretagogue, a biguanide, a thiazolidine, an alpha-glucosidase inhibitor, insulin, and an insulin analogue.

317. The method according to claim 316, wherein the sulfonylurea is selected from the group consisting of tolbutamide, chlorpropamide, tolazamide, azetohexamide, glyburide, glipizide, gliclazide, and glimepiride.

318. The method according to claim 316, wherein the insulin secretagogue is repaglinide or nateglinide.

319. The method according to claim 316, wherein the biguanide is metformin.

320. The method according to claim 316, wherein the thiazolidine is rosiglitazone or pioglitazone.

321. The method according to claim 316, wherein the alpha-glucosidase inhibitor is acarbose or miglitol.

322. The method of claim 314, wherein said agent is a protein or protein fragment according to claim 224.

323. The method of claim 314, wherein said agent is an antisense nucleic acid according to claim 251.

324. The method of claim 314, wherein said agent is an siRNA according to claim 254.

325. The method of claim 314, wherein said agent is a (poly)peptide or a small molecule drug which modulates the activity of the human Spinl1 protein of a human subject known not to have said medical condition, preferably the human Spinl1 protein according to SEQ ID NO:7 or an allelic variant thereof, said small molecule drug preferably having a molecular weight of no more than 2000 Dalton, preferably no more than 1500 Dalton, more preferably no more than 1000 Dalton, and most preferably no more than 500, 400, 300, or even 200 Dalton.

326. The method of claim 315, wherein the pharmaceutical composition or the agent is administered simultaneously with the one or more other compounds.

327. The method of claim 315, wherein the pharmaceutical composition or the agent is administered sequentially with the one or more other compounds.

328. The method of claim 303, wherein the pharmaceutical composition or the agent is capable of increasing leptin sensitivity, and wherein the medical condition is obesity.

329. The method according to claim 328, wherein the agent or the pharmaceutical composition is administered in conjunction with one or more compounds useful in the treatment of obesity.

330. The method according to claim 329, wherein the compound(s) is (are) selected from the group consisting of a lipase inhibitor and an appetite suppressant.

331. The method according to claim 330, wherein the lipase inhibitor is orlistat.

332. The method according to claim 330, wherein the appetite suppressant is sibutramine.

333. The method of claim 328, wherein said agent is a protein or protein fragment according to claim 224.

334. The method of claim 328, wherein said agent is an antisense nucleic acid according to claim 251.

335. The method of claim 328, wherein said agent is an siRNA according to claim 254.

336. The method of claim 328, wherein said agent is a (poly)peptide or a small molecule drug which modulates the activity of the human Spinl1 protein of a human subject known not to have said medical condition, preferably the human Spinl1 protein according to SEQ ID NO:7 or an allelic variant thereof, said small molecule drug preferably having a molecular weight of no more than 2000 Dalton, preferably no more than 1500 Dalton, more preferably no more than 1000 Dalton, and most preferably no more than 500, 400, 300, or even 200 Dalton.

337. The method of claim 329, wherein the pharmaceutical composition or the agent is administered simultaneously with the one or more other compounds.

338. The method of claim 329, wherein the pharmaceutical composition or the agent is administered sequentially with the one or more other compounds.

339. A method of identifying a binding partner of the Spinl1 protein, the method comprising
a) contacting a candidate binding partner agent with a wild-type mammalian Spinl1 protein, preferably the mouse Spinl1 protein or the human Spinl1 protein according to SEQ ID NO:3 and SEQ ID NO:7, respectively, under physiological conditions; and
b) determining whether binding of the candidate binding partner agent to said Spinl1 protein occurred.

340. The method of claim 339 wherein the binding of the candidate binding partner agent to said Spinl1 protein is determined by NMR technology.

341. A method of identifying an antagonist of the Spinl 1 protein, the method comprising
a) culturing mammalian cells in the presence or absence of a wild type mammalian Spinl1 protein, preferably the mouse Spinl1 protein or the human Spinl1 protein according to SEQ ID NO:3 and SEQ ID NO:7, respectively; and
b) determining whether an increase in size of endosomal and/or lysosomal compartments is observed in the presence of said wild type Spinl1 upon addition of a candidate antagonist agent to the cultured cells.

342. The method according to claim 341, wherein said cells show a reduced or no expression of endogenous Spinl1 protein, or carry a mutation in one or both alleles of their endogenous Spinl1 gene so that the allele is no longer capable of being expressed or that it encodes a protein according to claim 224.

343. The method according to claim 341, wherein the presence of the wild type mammalian Spinl1 protein in said cells is due to the introduction into said cells of a DNA sequence encoding and capable of expressing said wild type Spinl1 protein.

344. The method according to claim 341, further comprising
c) determining whether upon the addition of the candidate antagonist the size of endosomal and/or lysosomal compartments is affected in the mammalian cells used in step a) when cultured in the absence of wild type Spinl1 protein.

345. The method according to claim 341, further comprising the step of assigning Spinl1 antagonist function to the candidate antagonist if a reduced or no increase in size of endosomal and/or lysosomal compartments is observed in step b).

346. The method according to claim 341, further comprising the step of assigning Spinl1 antagonist function to a candidate antagonist if a reduced or no increase in size of endosomal and/or lysosomal compartments is observed in step b) and the size of endosomal and/or lysosomal compartments is unaffected or essentially unaffected in the mammalian cells in step c).

347. The method according to claim 342, wherein said cells are homozygous for said mutated endogenous Spinl1 allele.

348. The method according to claim 341 wherein said mammalian cells are adipocyte cells, particularly 3T3-L1 pre-adipocytes differentiated to adipocytes.

349. A method for identifying an agent capable of modulating leptin activity, the method comprising:
a) culturing cells in the presence or absence of an amount of leptin sufficient to induce STAT3 and/or AKT phosphorylation in said cells;
b) determining whether upon the addition of a candidate modulating agent the induction of STAT3 and/or AKT phosphorylation in the cells cultured in the presence of leptin is affected,
wherein said cells are hepatocytes or myoblasts or myocytes selected from the group of cells consisting of
i) myoblasts or myocytes which show a reduced or no expression of endogenous Spinl1 protein, or carry a mutation in one or both alleles of their endogenous Spinl 1 gene so that the allele is no longer capable of being expressed or that it encodes a protein according to claim 224;
ii) hepatocytes which show a reduced or no expression of endogenous Spinl1 protein, or carry a mutation in one or both alleles of their endogenous Spinl1 gene so that the allele is no longer capable of being expressed or that it encodes a protein according to claim 224;
iii) myoblasts or myocytes derived from a non-human vertebrate animal according to claim 257; and
iv) hepatocytes derived from a non-human vertebrate animal according to claim 257.

350. The method of claim 349, further comprising
c) determining whether upon the addition of said candidate modulating agent STAT3 and/or AKT phosphorylation is affected when the cells used in step a) are cultured in the absence of leptin.

351. The method of claim 349, wherein said myocytes are terminal differentiated myocytes.

352. The method according to claim 349, further comprising the step of assigning leptin activity inhibitory function to the candidate modulating agent, if upon the addition of the candidate modulating agent a decrease in the induction of STAT3 and/or AKT phosphorylation is obtained in step b).

353. The method according to claim 349, further comprising the step of assigning leptin activity promoting function to the candidate modulating agent, if upon the addition of the candidate modulating agent an increase in the induction of STAT3 and/or AKT phosphorylation is obtained in step b).

354. The method according to claim 349, further comprising the step of assigning leptin activity inhibitory function to the candidate modulating agent, if upon the addition of the candidate modulating agent a decrease in the induction of STAT3 and/or AKT phosphorylation is obtained in step b) and STAT3 and/or AKT phosphorylation is unaffected or essentially unaffected in step c).

355. The method according to claim 349, further comprising the step of assigning leptin activity promoting function to the candidate modulating agent, if upon the addition of the candidate modulating agent an increase in the induction of STAT3 and/or AKT phosphorylation is obtained in step b) and STAT3 and/or AKT phosphorylation is unaffected or essentially unaffected in step c).

356. The method of claim 349, wherein STAT3 and/or AKT phosphorylation is determined via antibodies specific to phosphorylated STAT3 and/or phosphorylated AKT, optionally via Western blotting of protein extracts of the cells to be analyzed.

357. A method for identifying an agent capable of modulating leptin sensitivity, the method comprising:
(a) culturing cells in the presence or absence of an amount of leptin sufficient to induce STAT3 and/or AKT phosphorylation in said cells;
b) determining whether upon the addition of a candidate modulating agent the kinetics of the induction of STAT3 and/or AKT phosphorylation in the cells cultured in the presence of leptin is affected,
wherein said cells are hepatocytes or myoblasts or myocytes selected from the group of cells consisting of
i) myoblasts or myocytes which show a reduced or no expression of endogenous Spinl1 protein, or carry a mutation in one or both alleles of their endogenous Spinl1 gene so that the allele is no longer capable of being expressed or that it encodes a protein according to claim 224;
ii) hepatocytes which show a reduced or no expression of endogenous Spinl1 protein, or carry a mutation in one or both alleles of their endogenous Spinl1 gene so that the allele is no longer capable of being expressed or that it encodes a protein according to claim 224;
iii) myoblasts or myocytes derived from a non-human vertebrate animal according to claim 257; and
iv) hepatocytes derived from a non-human vertebrate animal according to claim 257.

358. The method of claim 357, further comprising
c) determining whether upon the addition of said candidate modulating agent STAT3 and/or AKT phosphorylation is affected when the cells used in step a) are cultured in the absence of leptin.

359. The method of claim 357, wherein said myocytes are terminal differentiated myocytes.

360. The method according to claim 357, further comprising the step of assigning leptin sensitivity promoting function (i.e., leptin sensitizing function) to the candidate modulating agent, if the induction of STAT3 and/or AKT phosphorylation upon the addition of the candidate modulating agent in step b) is more rapid compared to the induction of STAT3 and/or AKT phosphorylation observed in the absence of the candidate modulating agent.

361. The method according to claim 357, further comprising the step of assigning leptin sensitivity decreasing function (i.e., leptin desensitizing function) to the candidate modulating agent, if the induction of STAT3 and/or AKT phosphorylation upon the addition of the candidate modulating agent in step b) is delayed compared to the induction of STAT3 and/or AKT phosphorylation observed in the absence of the candidate modulating agent.

362. The method according to claim 357, further comprising the step of assigning leptin sensitivity promoting function (i.e., leptin sensitizing function) to the candidate modulating agent, if the induction of STAT3 and/or AKT phosphorylation upon the addition of the candidate modulating agent in step b) is more rapid compared to the induction of STAT3 and/or AKT phosphorylation observed in the absence of the candidate modulating agent, and STAT3 and/or AKT phosphorylation is unaffected or essentially unaffected in step c).

363. The method according to claim 357, further comprising the step of assigning leptin sensitivity decreasing function (i.e., leptin desensitizing function) to the candidate modulating agent, if the induction of STAT3 and/or AKT phosphorylation is delayed upon the addition of the candidate modulating agent compared to the induction observed in the absence of the candidate modulating agent, and STAT3 and/or AKT phosphorylation is unaffected or essentially unaffected in step c).

364. The method of claim 357, wherein STAT3 and/or AKT phosphorylation is determined via antibodies specific to phosphorylated STAT3 and/or phosphorylated AKT, optionally via Western blotting of protein extracts of the cells to be analyzed.

365. A method for identifying an agent capable of increasing leptin sensitivity, the method comprising:
(a) culturing mammalian cells which show leptin-dependent STAT3 and AKT phosphorylation in the presence or absence of an amount of leptin which is in itself insufficient to induce STAT3 and/or AKT phosphorylation;
b) determining whether upon the addition of a candidate leptin sensitizing agent induction of STAT3 and/or AKT phosphorylation is obtained in the cells cultured in the presence of leptin.

366. The method of claim 365, further comprising
c) determining whether upon the addition of said candidate leptin sensitizing agent STAT3 and/or AKT phosphorylation is affected when the cells used in step a) are cultured in the absence of leptin.

367. The method of claim 365, further comprising the step of assigning leptin sensitizing function to the candidate agent, if upon the addition of the candidate sensitizing agent an induction of STAT3 and/or AKT phosphorylation is obtained in step b).

368. The method of claim 365, further comprising the step of assigning leptin sensitizing function to the candidate agent, if upon the addition of the candidate sensitizing agent an induction of STAT3 and/or AKT phosphorylation is obtained in step b), and STAT3 and/or AKT phosphorylation is unaffected or essentially unaffected in step c).

369. The method according to claim 365, wherein the cells are hepatocytes or myocytes.

370. The method of claim 369, wherein the myocytes are derived from db/db mice and express a functional leptin receptor, particularly a leptin receptor fusion with a reporter polypeptide, due to transfection of the cells with a DNA sequence encoding said functional leptin receptor.

371. The method according to claim 365, wherein STAT3 and/or AKT phosphorylation is determined via antibodies specific to phosphorylated STAT3 and/or phosphorylated AKT, optionally via Western blotting of protein extracts of the cells to be analyzed.

372. The method according to claim 339, wherein the agent is selected from the group consisting of
a) a peptide or polypeptide;
b) a nucleic acid (including a peptide nucleic acid); and
c) a small molecule having a molecular weight of no more than 2000 Dalton, preferably no more than 1500 Dalton, more preferably no more than 1000 Dalton, and most preferably no more than 500, 400, 300, or even 200 Dalton;
the method obtionally comprising the step of preparing or synthesizing the agent, and further optionally comprising the step of formulating the agent into a pharmaceutical composition.

373. The method according to claim 341, wherein the agent is selected from the group consisting of
a) a peptide or polypeptide;
b) a nucleic acid (including a peptide nucleic acid); and
c) a small molecule having a molecular weight of no more than 2000 Dalton, preferably no more than 1500 Dalton, more preferably no more than 1000 Dalton, and most preferably no more than 500, 400, 300, or even 200 Dalton;
the method obtionally comprising the step of preparing or synthesizing the agent, and further optionally comprising the step of formulating the agent into a pharmaceutical composition.

374. The method according to claim 349, wherein the agent is selected from the group consisting of
a) a peptide or polypeptide;
b) a nucleic acid (including a peptide nucleic acid); and
c) a small molecule having a molecular weight of no more than 2000 Dalton, preferably no more than 1500 Dalton, more preferably no more than 1000 Dalton, and most preferably no more than 500, 400, 300, or even 200 Dalton;
the method obtionally comprising the step of preparing or synthesizing the agent, and further optionally comprising the step of formulating the agent into a pharmaceutical composition.

375. The method according to claim 357, wherein the agent is selected from the group consisting of
a) a peptide or polypeptide;
b) a nucleic acid (including a peptide nucleic acid); and
c) a small molecule having a molecular weight of no more than 2000 Dalton, preferably no more than 1500 Dalton, more preferably no more than 1000 Dalton, and most preferably no more than 500, 400, 300, or even 200 Dalton;
the method obtionally comprising the step of preparing or synthesizing the agent, and further optionally comprising the step of formulating the agent into a pharmaceutical composition.

376. The method according to claim 365, wherein the agent is selected from the group consisting of
a) a peptide or polypeptide;
b) a nucleic acid (including a peptide nucleic acid); and
c) a small molecule having a molecular weight of no more than 2000 Dalton, preferably no more than 1500 Dalton, more preferably no more than 1000 Dalton, and most preferably no more than 500, 400, 300, or even 200 Dalton;
the method optionally comprising the step of preparing or synthesizing the agent, and further optionally comprising the step of formulating the agent into a pharmaceutical composition.

377. An agent identified or identifiable by a method according to claim 339

378. An agent identified or identifiable by a method according to claim 341.

379. An agent identified or identifiable by a method according to claim 349.

380. An agent identified or identifiable by a method according to claim 357.

381. An agent identified or identifiable by a method according to claim 365.
